(19) United States
(12) Patent Application Publication
(10) Pub. No.: US 2008/0014191 A1
(43) Pub. Date: Jan. 17, 2008

(54) Treatment of Protein Misfolding

(75) Inventors: William E. Balch, San Diego, CA (US); Paul G. LaPointe, San Diego, CA (US); John D. Venable, Solana Beach, CA (US); Xiaodong Wang, Sylvania, OH (US); John R. Yates III, San Diego, CA (US)

Correspondence Address: BIOTACTICA, LLC, 10733 Sunset Office Drive, Suite 261, ST. LOUIS, MO, 63127, US

(73) Assignee: The Scripps Research Institute, La Jolla, CA (US)

(21) Appl. No.: 11/751,528

(22) Filed: May 21, 2007

Related U.S. Application Data
(60) Provisional application No. 60/801,840, filed on May 19, 2006.
(60) Provisional application No. 60/815,494, filed on Jun. 21, 2006.
(60) Provisional application No. 60/859,890, filed on Nov. 17, 2006.

(51) Int. Cl. A61K 31/713; A61K 39/395; A61P 25/16; A61P 25/28; C07H 21/02; C07K 16/00; C12N 1/00; C12N 15/63; C12Q 1/68

(52) U.S. Cl. 424/130.1; 435/243; 435/320.1; 435/6; 514/44; 514/789; 530/387.1; 536/23.1

(57) ABSTRACT

The present invention is directed to preventing the consequences of the misfolding of proteins, such as those associated with protein folding diseases. Provided are methods of treatment that involve administering an agent that decreases the level of the heat shock protein ATPase Aha1 and/or related molecules with similar function. Such methods can result in the rescue of folding, trafficking, and function of proteins with suboptimal folding kinetics. Also provided are screening methods to identify agents for the treatment of protein misfolding disease

### CROSS-REFERENCE TO RELATED APPLICATIONS

This application claims priority from U.S. Provisional Application Ser. No. 60/801,840 filed on May 19, 2006, U.S. Provisional Application Ser. No. 60/815,494 filed on Jun. 21, 2006, and U.S. Provisional Application Ser. No. 60/859,890 filed on Nov. 17, 2006, each of which is incorporated herein by reference in its entirety.

### STATEMENT REGARDING FEDERALLY SPONSORED RESEARCH OR DEVELOPMENT

This invention was made in part with Government support under National Institutes of Health Grants GM42336 and GM45678/NIH RR11823. The Government has certain rights in the invention.

### INCORPORATION-BY-REFERENCE OF MATERIAL SUBMITTED ON A COMPACT DISC

The Sequence Listing, which is a part of the present disclosure, includes a computer file “Sequence Listing_ST25.TXT” generated by U.S. Patent & Trademark Office Patent In Version 3.4 software comprising nucleotide and/or amino acid sequences of the present invention. The subject matter of the Sequence Listing is incorporated herein by reference in its entirety.

### FIELD

The present invention generally relates to methods for treatment of protein misfolding diseases. In particular, the present invention concerns methods of treatment using modulators of the gene Activator of Heat Shock Protein 90 ATPase (Aha). For example, the invention provides compositions and methods of treating disorders associated with undesired Aha activity by administering double-stranded RNA (dsRNA) which down-regulates the expression of Aha.

### INTRODUCTION

The endoplasmic reticulum (ER) is a specialized folding environment in which nearly one-third of the proteins encoded by a eukaryotic genome are translocated and folded as either lumenal secreted proteins or transmembrane proteins. Proteins are exported from the ER by the concatamer complex II (COPII) machinery which generates transport vesicles for delivery of cargo to the Golgi (Lee et al., Annu. Rev Cell Dev. Biol. 20, 87 (2004)). The ER-associated folding (ERAF) pathways are also coordinated with ER-associated degradation (ERAD) pathways whereby misfolded proteins are targeted for translocation to the cytosolic proteasome system (Wegele et al., Rev Physiol Biochem Pharmacol 151, 1 (2004); Young et al., Trends Biochem. Sci. 28, 541 (2003)).

Numerous misfolding diseases occur in which variants of either lumenal or transmembrane cargo do not fold properly, fail to engage the COPII export machinery and are degraded in the ER resulting in loss of function phenotype. Cystic fibrosis (CF) is an inherited childhood disease primarily triggered by defective folding and export of CF transmembrane conductance regulator (CFTR; a multi-domain cAMP-regulated chloride channel found in the apical membrane of polarized epithelia lining many tissues) from the ER (Riordan, Annu. Rev. Physiol. 67, 701 (2005)). CFTR consists of two transmembrane domains (TMD1 and 2), separated biosynthetically by cytosol oriented N- and C-terminal domains, and the NBD1, R and NBD2 domains that regulate channel conductance. Transport of CFTR involves chaperones directing folding and export from the ER (Amaral, J. Mol. Neurosci. 23, 41 (2004); Wang et al., J. Struct. Biol. 146, 44 (2004)) as well as adaptor proteins that direct trafficking from the trans Golgi (Cheng et al., J. Biol. Chem. 280, 3731 (2005)) and recycling through endocytic pathways to maintain the proper level of chloride channel activity at the cell surface (Gentzsch et al., Mol. Biol. Cell 15, 2684 (2004); Swiatecka-Urban et al., J. Biol. Chem. 280, 36762 (2005)).

Over 90% of CF patients carry at least one allele of the Phe 508 deletion (ΔF508) in the cytosolic NBD1 ATP-binding domain of CFTR leading to severe forms of disease. ΔF508 disrupts the folding of CFTR in the ER (Qu et al., J. Bioenerg. Biomembr. 29, 483 (1997); Riordan, supra). The folding of ΔF508 NBD1 is reported to be kinetically impaired (Qu et al., supra; Qu et al., J. Biol. Chem. 272, 15739 (1997); Qu and Thomas, J. Biol. Chem. 271, 7261 (1996)). As a consequence of this energetic defect in folding, ΔF508 fails to achieve a wild-type fold in the ER, fails to engage the COPII ER export machinery (Wang et al., supra) and is targeted for ER-associated degradation (ERAD) (Nishikawa et al., J Biochem (Tokyo) 137, 551 (2005)). Thus, it would be desirable to provide some means for preventing the consequences of the misfolding of ΔF508 CFTR in the treatment of CF.

Activator of Heat Shock Protein 90 ATPase 1 (Aha1) is an activator of the ATPase-activity of Hsp90 and is able to stimulate the inherent activity of yeast Hsp90 by 12-fold and human Hsp90 by 50-fold (Panaretou, B., et al., Mol. Cell 2002, 10:1307-1318). Biochemical studies have shown that Aha1 binds to the middle region of Hsp90 (Panaretou et al., 2002, supra, Lotz, G. P., et al., J. Biol. Chem. 2003, 278:17228-17235), and recent structural studies of the Aha1-Hsp90 core complex suggest that the co-chaperone promotes a conformational switch in the middle segment catalytic loop (370-390) of Hsp90 that releases the catalytic Arg380 and facilitates its interaction with ATP in the N-terminal nucleotide-binding domain (Meyer, P., et al., EMBO J. 2004, 23:511-519).

The molecular chaperone Heat shock protein 90 (Hsp90) is responsible for the in vivo activation or maturation of specific client proteins (Picard, D., Cell Mol. Life. Sci. 2002, 59:1640-1648; Pearl, L. H., and Prodromou, C., Adv. Protein Chem. 2002, 59:157-185; Pratt, W. B., and Toft, D. O., Exp. Biol. Med. 2003, 228:111-133; Prodromou, C., and Pearl, L. H., Curr. Cancer Drug Targets 2003, 3:301-323). Crucial to such activation is the essential ATPase activity of Hsp90 (Panaretou, B., et al., EMBO J. 1998, 17:4829-4836), which drives a conformational cycle involving transient association of the N-terminal nucleotide-binding domains within the Hsp90 dimer (Prodromou, C., et al., EMBO J. 2000, 19:4383-4392).

As a molecular chaperone, HSP90 promotes the maturation and maintains the stability of a large number of conformationally labile client proteins, most of which are involved in biologic processes that are often deranged within tumor cells, such as signal transduction, cell-cycle progression and apoptosis. As a result, and in contrast to other molecular targeted therapeutics, inhibitors of HSP90 achieve promising anticancer activity through simultaneous disruption of many oncogenic substrates within cancer cells (Whitesell L, and Dai C., Future Oncol. 2005; 1:529-540; WO 03/067262). Furthermore, HSP90 has been implicated in the degradation of Cystic Fibrosis Transmembrane Conductance Regulator (CFTR). Mutations in the CFTR gene lead to defective folding and ubiquination of the protein as a consequence of HSP90 ATPase activity. Following ubiquitination, CFTR is degraded before it can reach its site of activity. Lack of active CFTR then leads to the development of cystic fibrosis in human subjects having such mutation. Therefore, the inhibition of HSP90 activity may be beneficial for subjects suffering from cancer or Cystic Fibrosis.

Hsp90 constitutes about 1-2% of total cellular protein (Pratt, W. B., Annu. Rev. Pharmacol. Toxicol. 1997, 37:297-326), and the inhibition of such large amounts of protein by means of an antagonist or inhibitor would potentially require the introduction of excessive amounts of the inhibitor or antagonist into a cell. An alternative approach is the inhibition of activators of HSP90's ATPase activity, such as Aha1, which are present in smaller amounts. By downregulating the amount of Aha1 present in the cell, the activity of HSP90 may be lowered substantially.

Significant sequence homology exists between Homo sapiens (NM_{—}012111.1), Mus musculus (NM_{—}146036.1) and Pan troglodytes (XM_{—}510094.1) Aha 1. A clear rattus norvegicus homologue of Aha 1 has not been identified; however, there is a Rattus norvegicus (XM_{—}223680.3) gene which has been termed activator of heat shock protein ATPase homolog 2 (Ahsa 2) on the basis of its sequence homology to yeast Ahsa 2. Its sequence is homologous to mus musculus RIKEN cDNA 1110064P04 gene (NM_{—}172391.3), which is in turn similar in sequence to Mus musculus Aha 1 except for N-terminal truncation. A homo sapiens Ahsa 2 (NM_{—}152392.1) has also been predicted, but sequence homology is limited. The functions of these latter three genes have not been sufficiently elucidated. However, there exists one region in which all of the above sequences are identical, and which may be used as the target for RNAi agents. It may be advantageous to inhibit the activity of more than one Aha gene.

Recently, dsRNA have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

Despite significant advances in the field of RNAi and advances in the treatment of pathological processes mediated by HSP90, there remains a need for agents that can selectively and efficiently attenuate HSP90 ATPase activity, for example, by using the cell's own RNAi machinery. Such agents can possess both high biological activity and in vivo stability, and may effectively inhibit expression of a target Aha gene, such as Aha1, for use in treating pathological processes mediated directly or indirectly by Aha expression, e.g., Aha1 expression. Such agents may also effectively inhibit an activity of functional Aha1 protein, e.g., heat shock protein ATPase activator activity.

### SUMMARY

Accordingly, the present inventors have succeeded in discovering that decreasing levels of functional Aha1, a heat shock protein (Hsp) co-chaperone and ATPase activator, can result in energetic stabilization of the ΔF508 variant of CFTR, associated with CF. This results in rescue of folding, trafficking, and function of ΔF508.

Thus, the present invention includes compositions and methods for treating a disease resulting from protein misfolding. The compositions can generally comprise a dsRNA, vector, short hairpin RNA (shRNA), small molecule, antibody, antisense nucleic acid, aptamer, ribozyme, and any combination thereof for inhibiting functional Aha protein expression in a cell.

For example, the dsRNA can comprise a sense strand and an antisense strand, wherein said antisense strand comprises a region of complementarity having a sequence substantially complementary to an Aha target sequence, wherein said target sequence is less than 30 nucleotides in length, wherein said sense strand is substantially complimentary to said antisense strand, and wherein said dsRNA, upon contact with a cell expressing functional Aha protein, inhibits functional Aha protein expression by at least 20%. In various aspects, the Aha target sequence can comprise a sequence selected from the group consisting of SEQ ID NOs: 12-56. In another aspect, the dsRNA can comprises a sense strand having a sequence selected from the group consisting of SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, and SEQ ID NO: 145; and an antisense strand complementary to the sense strand having a sequence selected from the group consisting of SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, and SEQ ID NO: 146.

In another example, the vector for expressing a shRNA for inhibiting functional Aha1 expression in a cell can comprise a sense strand, a hairpin linker, and an antisense strand. In various aspects, the sense strand can comprise a region of complementarity having a sequence substantially complementary to an Aha target sequence, wherein said target sequence is less than 30 nucleotides in length, the antisense strand can be substantially complimentary to said sense strand, and the dsRNA, upon contact with a cell expressing functional Aha protein, can inhibit functional Aha protein expression by at least 20%. In various aspects, the Aha target sequence can comprise a sequence selected from the group consisting of SEQ ID NOs: 12-56. In another aspect, the vector can comprise a sense strand having a sequence selected from the group consisting of SEQ ID NO: 147, SEQ ID NO: 149, and SEQ ID NO: 151; and an antisense strand having a sequence selected from the group consisting of SEQ ID NO: 148, SEQ ID NO: 150, and SEQ ID NO: 152.

In another example, the shRNA for inhibiting functional Aha1 protein expression in a cell, can comprise a region of complementarity having a sequence substantially complementary to an Aha target sequence, wherein said target sequence is less than 30 nucleotides in length, and wherein said shRNA, upon contact with a cell expressing functional Aha protein, inhibits functional Aha protein expression by at least 20%. In various aspects, the Aha target sequence can comprise a sequence selected from the group consisting of SEQ ID NOs: 12-56. In another aspect, the shRNA can comprise a sequence selected from the group consisting of SEQ ID NO: 153, SEQ ID NO: 154, and SEQ ID NO: 155;

The invention also provides a cell or cell population comprising the dsRNA, vector and/or shRNA.

In another example, the antibody can specifically bind functional Aha1, the Hsp90 ATPase binding site for functional Aha1, and/or the functional Aha1-Hsp90 ATPase complex.

In yet another example, the agent can include any combination of a small molecule, an antibody, an antisense nucleic acid, an aptamer, a dsRNA, and a ribozyme.

The invention also provides a method of treating a disease associated with misfolding of a protein. The method can comprise administering to a subject in need thereof a therapeutically effective amount of at least one agent that decreases intracellular levels of functional Aha1 protein. In various aspects, the agent can be selected from the group consisting of a small molecule, an antibody, an antisense nucleic acid, an aptamer, an siRNA, a ribozyme, and combinations thereof. In various aspects, the disease can include cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease. In another aspect, the misfolded protein can be a misfolded CFTR. In yet another aspect, the misfolded protein can be a ΔF508 protein.

The method can also include administering to a subject in need thereof a therapeutically effective amount of at least one dsRNA inhibitor of functional Aha1 expression, said dsRNA comprising a sense strand and an antisense strand. In various aspects, the antisense strand can comprise a region of complementarity having a sequence substantially complementary to an Aha target sequence, wherein said target sequence is less than 30 nucleotides in length, the sense strand is substantially complimentary to said antisense strand, and the dsRNA, upon contact with a cell expressing functional Aha protein, inhibits functional Aha protein expression by at least 20%. In various aspects, the disease can include cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease. In another aspect, the misfolded protein can be a misfolded CFTR. In yet another aspect, the misfolded protein can be a ΔF508 protein.

The method can also include administering to a subject in need thereof a therapeutically effective amount of at least one dsRNA inhibitor of functional Aha1 expression. In various aspects, the dsRNA inhibitor can comprise a sequence selected on the basis of a) the dsRNA comprising a sense strand sequence of about 19 nucleotides to about 25 nucleotides and an antisense strand sequence of about 19 nucleotides to about 25 nucleotides; and b) the sense strand sequence or antisense strand sequence comprises no more than 15 contiguous nucleotides identical to a contiguous sequence comprised by a 5′ untranslated region, a 3′ untranslated region, an intron or an exon of any gene or mRNA other than functional Aha1. In various aspects, the disease can include cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease. In another aspect, the misfolded protein can be a misfolded CFTR. In yet another aspect, the misfolded protein can be a ΔF508 protein.

A method of the invention can also include screening an agent for treating a disease associated with misfolding of a protein. In various aspects, the method can comprise providing a cell or cell population expressing functional Aha1; administering a candidate agent to the cell or cell population; quantifying functional Aha1 activity in the cell or cell population; and determining whether the candidate agent decreases functional Aha1 activity in the cell or cell population, whereby a decrease in functional Aha1 activity is indicative of reducing misfolding of the protein. In various aspects, the candidate agent can be a dsRNA which inhibits functional Aha1 expression. In another aspect, the dsRNA can comprise a) a sequence of from about 19 nucleotides to about 25 nucleotides, and b) the sequence comprises no more than 15 contiguous nucleotides identical to a contiguous sequence comprised by a 5′ untranslated region, a 3′ untranslated region, an intron or an exon of any gene or mRNA other than an Aha gene or mRNA. In various aspects, the Aha gene or mRNA is a human Aha gene or mRNA. In another aspect, the disease can be selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease. In another aspect, the misfolded protein can be selected from the group consisting of a misfolded CFTR, a misfolded fibrillin, a misfolded alpha galactosidase, a misfolded beta glucocerebrosidase, a misfolded rhodopsin, aggregated an amyloid beta and tau, an aggregated amylin, an aggregated alpha synuclein and an aggregated prion. In yet another aspect, the misfolded protein can be a misfolded CFTR. And in another aspect, the misfolded protein can be a ΔF508 protein.

The screening method can also comprise providing a cell or cell population which expresses functional Aha1; administering a candidate agent to the cell or cell population; quantifying Hsp90/ADP complex, Hsp90/ATP complex or a combination thereof in the cell or cell population; and determining whether the candidate agent decreases the quantity of Hsp90/ADP complex, Hsp90/ATP complex or the combination thereof in the cell or cell population, whereby a decrease in quantity of Hsp90/ADP complex or Hsp90/ATP complex is indicative of decreasing misfolding of the protein. In various aspects, the candidate agent can be a dsRNA which inhibits functional Aha1 expression. In another aspect, the dsRNA can comprises a) a sequence of from about 19 nucleotides to about 25 nucleotides, and b) the sequence comprises no more than 15 contiguous nucleotides identical to a contiguous sequence comprised by a 5′ untranslated region, a 3′ untranslated region, an intron or an exon of any gene or mRNA other than an Aha gene or mRNA. In yet another aspect, the Aha gene or mRNA can be a human Aha gene or mRNA. In various aspects, the disease can be selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease. In another aspect, the misfolded protein can be selected from the group consisting of a misfolded CFTR, a misfolded fibrillin, a misfolded alpha galactosidase, a misfolded beta glucocerebrosidase, a misfolded rhodopsin, aggregated an amyloid beta and tau, an aggregated amylin, an aggregated alpha synuclein and an aggregated prion. In yet another aspect, the misfolded protein can be a misfolded CFTR. In another aspect, the misfolded protein can be a ΔF508 protein.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Depiction of the CFTR interactome.

FIG. 2. (A) Depiction of the ER folding network, and (B) immunoblot depicting protein expression levels in WT and ΔF508 expressing cells.

FIG. 3. Series of bar graphs depicting the effect of the Hsp90 co-chaperone p23 on folding and export of ΔF508 from the ER.

FIG. 4. Series of bar graphs depicting the effect of the Hsp90 co-chaperone FKBP8 on folding and export of ΔF508 from the ER.

FIG. 5. Series of bar graphs depicting the effect of the Hsp90 co-chaperone HOP on folding and export of ΔF508 from the ER.

FIG. 6. Series of bar graphs illustrating that ΔF508 export to the cell surface can be rescued by downregulation of functional Aha1.

FIG. 7. Line and scatter plot and a bar graph showing the effect of dsRNA Aha1 on iodide efflux by the CFBE41o-cell line.

FIG. 8. Series of depictions of Hsp90 chaperone/co-chaperone interactions directing CFTR folding.

FIG. 9. Illustration (using immunoblot) of effects of dsRNA Aha1 on Hsp90.

### DETAILED DESCRIPTION

Abbreviations and Definitions

To facilitate understanding of the invention, a number of terms and abbreviations as used herein are defined below as follows:

“G,” “C,” “A”, “T” and “U” (irrespective of whether written in capital or small letters) each generally stand for a nucleotide that contains guanine, cytosine, adenine, thymine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, thymine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine.

The terms “functional Aha1 protein” or “functional Aha1” as used herein are intended to include a human Aha1 polypeptide (SEQ ID NO: 4) having heat shock protein ATPase activator activity as well as molecules related to Aha1 having heat shock protein ATPase activator activity. Such molecules related to human Aha1 include polypeptides having heat shock protein ATPase activator activity and at least 80% homology to functional Aha1. For example, related molecules can have 80%, 81%, 82%, 83%, 84%, 85%, 86%, 87%, 88%, 89%, 90%, 91%, 92%, 93%, 94%, 95%, 96%, 97%, 98%, or 99% homology to human Aha1 and can have heat shock protein ATPase activator activity. Such molecules can include, for example, SEQ ID NO: 5, SEQ ID NO: 6, SEQ ID NO: 7; and SEQ ID NO: 8. In addition, such molecules related to human Aha1 include polypeptides having longer or shorter amino acid sequences and having heat shock protein ATPase activator activity.

Heat shock protein ATPase activator activity may be determined using standard assays, for example, by determining the production of inorganic phosphate (Pᵢ) by Hsp90. Pᵢ production may be determined, for example, by measuring or determining the generation or depletion of a reporter molecule. One such method utilizes a regenerating ATPase assay using a pyruvate kinase/lactate dehydrogenase linked assay in which the generation of Pᵢ can be measured spectrophotometrically (Ali et al., Biochemistry (1993) 32:2717-2724). Other spectrophotometric methods include those described by Lanzetta et al. (1979) Anal. Biochem. 100, 95-97; Lill et al., (1990) Cell 60, 271-280; and Cogan et al., Anal. Biochem. (1999) 271:29-35. Those of skill in the art will recognize other methods of measuring heat shock protein ATPase activator activity.

As used herein, “Aha gene” refers to an Activator of Heat Shock Protein 90 ATPase genes that can express a functional Aha1 protein. “Aha1” refers to Activator of Heat Shock Protein 90 ATPase 1 genes, non-exhaustive examples of which are found under Genbank accession numbers NM_{—}012111.1 (Homo sapiens), NM_{—}146036.1 (Mus musculus), and XM_{—}510094.1 (Pan troglodytes).

As used herein, “target sequence” refers to a contiguous portion of the nucleotide sequence of an mRNA molecule formed during the transcription of an Aha gene, including mRNA that is a product of RNA processing of a primary transcription product. The target sequence of any given RNAi agent of the invention means an mRNA-sequence of X nucleotides that is targeted by the RNAi agent by virtue of the complementarity of the antisense strand of the RNAi agent to such sequence and to which the antisense strand may hybridize when brought into contact with the mRNA, wherein X is the number of nucleotides in the antisense strand plus the number of nucleotides in a single-stranded overhang of the sense strand, if any.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding Aha1). For example, a polynucleotide is complementary to at least a part of an Aha1 mRNA if the sequence is substantially complementary to a non-interrupted portion of an mRNA encoding Aha1.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop” and the entire structure is referred to as a “short hairpin RNA” or “shRNA”. Where the two strands are connected covalently by means other than an uninterrupted chain of nucleotides between the 3′-end of one strand and the 5′-end of the respective other strand forming the duplex structure, the connecting structure is referred to as a “linker”. In various aspects, the linker can include the sequences AUG, CCC, UUCG, CCACC, CTCGAG, AAGCUU, CCACACC, and UUCAAGAGA. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that has no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus. In certain aspects of the invention, the mismatches can be located within 6, 5, 4, 3, or 2 nucleotides of the 5′ terminus of the antisense strand and/or the 3′ terminus of the sense strand.

The term “sense strand,” as used herein, refers to the strand of a dsRNA that includes a region that is substantially complementary to a region of the antisense strand.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

The terms “decrease, decreased or decreasing levels” as used herein are intended to include inhibiting Aha1 heat shock protein ATPase activator activity and reducing the amount of functional Aha1 protein in a cell. For example, an antibody or dsRNA can decrease the level of functional Aha1 protein by interfering with or silencing heat shock protein ATPase activator activity without removing the Aha1 protein from the cell. In another example, a ribozyme can cleave the functional Aha1 protein to reduce the amount of whole Aha1 protein in the cell. In another example, a dsRNA can silence the expression an Aha gene, e.g. an Aha1 gene, to reduce the amount of mRNA transcribed from the Aha gene.

The terms “silence” and “inhibit the expression of”, in as far as they refer to an Aha gene, e.g. an Aha1 gene, herein refer to the at least partial suppression of the expression of an Aha gene, e.g. an Aha1 gene, as manifested by a reduction of the amount of mRNA transcribed from an Aha gene which may be isolated from a first cell or group of cells in which an Aha gene is transcribed and which has or have been treated such that the expression of an Aha gene is inhibited, as compared to a second cell or group of cells substantially identical to the first cell or group of cells but which has or have not been so treated (control cells). In various aspects of the invention, the cells can be HeLa or MLE 12 cells. The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to Aha gene transcription, e.g. the amount of protein encoded by an Aha gene which is secreted by a cell, or found in solution after lysis of such cells, or the number of cells displaying a certain phenotype, e.g. apoptosis or cell surface CFTR. In principle, Aha gene silencing may be determined in any cell expressing the target, either constitutively or by genomic engineering, and by any appropriate assay. However, when a reference is needed in order to determine whether a given dsRNA inhibits the expression of an Aha gene by a certain degree and therefore is encompassed by the instant invention, the assays provided in the Examples below shall serve as such reference.

For example, in certain instances, expression of an Aha gene, e.g. an Aha1 gene, is suppressed by at least about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% by administration of the double-stranded oligonucleotide of the invention. In various aspects, an Aha gene, e.g. an Aha1 gene, is suppressed by at least about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% or 80% by administration of the double-stranded oligonucleotide of the invention. In various aspects, an Aha gene, e.g. an Aha1 gene, is suppressed by at least about 81%, 82%, 83%, 84%, 85%, 86%, 87%, 88%, 89%, 90%, 91%, 92%, 93%, 94%, 95%, 96%, 97%, 98%, 99% or 100% by administration of the double-stranded oligonucleotide of the invention.

As used herein in the context of Aha expression, e.g. Aha1 expression, the terms “treat”, “treatment”, and the like, refer to relief from or alleviation of pathological processes mediated by Aha expression. In the context of the present invention insofar as it relates to any of the other conditions recited herein below (other than pathological processes mediated by Aha expression), the terms “treat”, “treatment”, and the like mean to relieve or alleviate at least one symptom associated with such condition, or to slow or reverse the progression of such condition.

As used herein, the phrases “therapeutically effective amount” and “prophylactically effective amount” refer to an amount that provides a therapeutic benefit in the treatment, prevention, or management of pathological processes mediated by Aha expression or an overt symptom of pathological processes mediated by Aha expression. The specific amount that is therapeutically effective can be readily determined by ordinary medical practitioner, and may vary depending on factors known in the art, such as, e.g. the type of pathological processes mediated by Aha expression, the patient's history and age, the stage of pathological processes mediated by Aha expression, and the administration of other anti-pathological processes mediated by Aha expression agents.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

Treatment of Protein Misfolding

The present invention provides methods for the treatment of misfolding diseases by decreasing levels of functional Aha1 protein (e.g., SEQ ID NO: 4) and/or other related molecules with similar function, as well as methods for the screening of agents useful for treatment of protein misfolding diseases.

The technology described herein is based in part on the observation that decreased levels of functional Aha1, an Hsp90 ATPase activator, can markedly stabilize ΔF508 (ΔF508 polypeptide of SEQ ID NO: 3), a mutant of CFTR (CFTR mRNA of SEQ ID NO: 1; CFTR polypeptide of SEQ ID NO: 2) characterized by a phenylalanine deletion at 508, in a folded state that is accessible to the COPII export machinery for transport to the cell surface. Various therapeutic strategies described herein are directed to downregulation of functional Aha1 and/or other related molecules with similar function, salvage of mutant CFTR misfolding, rescue of Hsp90-mediated trafficking to the cell surface, and at least partially restoration of channel functions in a subject.

Agents that Decrease Functional Aha1

Agents that decrease levels of functional Aha1 and/or other related molecules with similar function, can target functional Aha1 and/or Hsp90 ATPase such that binding to one component or both of the components by the agent effects a decrease in heat shock protein ATPase activator activity, the activation state of Hsp90 ATPase, and/or the activity level of activated Hsp90 ATPase, consequently resulting in stabilization of misfolded proteins. A crystal structure of the complex between Hsp90 and Aha1 has been reported (Meyer et al. (2004) EMBO J. 23, 511-519). Given a structural and mechanistic understanding of Aha1, Hsp90 ATPase, and the binding complex formed between the two, it is within the skill of the art to design agents that bind, for example ionically or covalently, to one or both components and thereby reduce the activation of Hsp90 ATPase by functional Aha1.

The various classes of agents for use herein as agents that decrease levels of functional Aha1 and/or related molecules with similar function, generally include, but are not limited to, RNA interference molecules, antibodies, small inorganic molecules, antisense oligonucleotides, and aptamers.

RNA interference (RNAi) can be used to decrease the levels of functional Aha1 (and/or other related molecules with similar functions) (see e.g., Examples 8-10). RNAi methods can utilize double stranded RNAs, for example, small interfering RNAs (siRNA), short hairpin RNA (shRNA), and micro RNAs (miRNA). The following discussion will focus on dsRNA generally, but one skilled in the art will recognize many approaches are available for other RNAi molecules, such as miRNA. RNAi molecules, specific for functional Aha1 and/or other related molecules of similar function, are also commercially available from a variety of sources (e.g., Silencer® In Vivo Ready dsRNAs, Aha1 dsRNA ID#s 1136422, 136423, 36424, 19683, 19588, 19773, Ambion, Tex.; Sigma Aldrich, MO; Invitrogen, CA).

Several dsRNA molecule design programs using a variety of algorithms are known to the art (see e.g., Cenix algorithm, Ambion; BLOCK-iT™ RNAi Designer, Invitrogen; dsRNA Whitehead Institute Design Tools, Bioinoformatics & Research Computing). Traits influential in defining optimal dsRNA sequences include G/C content at the termini of the dsRNAs, Tₘ of specific internal domains of the dsRNA, dsRNA length, position of the target sequence within the CDS (coding region), and nucleotide content of the 3′ overhangs.

Administration of dsRNA molecules specific for functional Aha1, and/or other related molecules with similar functions, can effect the RNAi-mediated degradation of the target (e.g., Aha1) mRNA. For example, a therapeutically effective amount of dsRNA specific for Aha1 can be adminstered to patient in need thereof to treat a protein misfolding disease. In one aspect, the dsRNA that effects decreased levels of functional Aha1 has a nucleotide sequence including SEQ ID NOs: 57-146 (see Table 2 below).

Generally, an effective amount of dsRNA molecule can comprise an intercellular concentration at or near the site of misfolding from about 1 nanomolar (nM) to about 100 nM, and in various aspects from about 2 nM to about 50 nM, and in other aspects from about 2.5 nM to about 10 nM. It is contemplated that greater or lesser amounts of dsRNA can be administered.

The dsRNA can be administered to the subject by any means suitable for delivering the RNAi molecules to the cells of interest. For example, dsRNA molecules can be administered by gene gun, electroporation, or by other suitable parenteral or enteral administration routes, such as intravitreous injection. RNAi molecules can also be administered locally (lung tissue) or systemically (circulatory system) via pulmonary delivery. A variety of pulmonary delivery devices can be effective at delivering functional Aha1-specific RNAi molecules to a subject (see below). RNAi molecules can be used in conjunction with a variety of delivery and targeting systems, as described in further detail below. For example, dsRNA can be encapsulated into targeted polymeric delivery systems designed to promote payload internalization.

The dsRNA can be targeted to any stretch of less than 30 contiguous nucleotides, generally about 19-25 contiguous nucleotides, in the functional Aha1 (or other related molecule with similar function) mRNA target sequences, e.g. SEQ ID NOs: 12-56 (see Table 1 below). Searches of the human genome database (BLAST) can be carried out to ensure that selected dsRNA sequence will not target other gene transcripts. Techniques for selecting target sequences for dsRNA are known in the art (see e.g., Reynolds et al. (2004) Nature Biotechnology 22(3), 326-330). Thus, the sense strand of the present dsRNA can comprise a nucleotide sequence identical to any contiguous stretch of about 19 to about 25 nucleotides in the target mRNA of functional Aha1 (or related molecule with similar function). Generally, a target sequence on the target mRNA can be selected from a given cDNA sequence corresponding to the target mRNA, for example, beginning 50 to 100 nt downstream (i.e., in the 3′ direction) from the start codon. The target sequence can, however, be located in the 5′ or 3′ untranslated regions, or in the region nearby the start codon.

The dsRNA of the invention can comprise an RNA strand (the antisense strand) having a region which is less than 30 nucleotides in length, generally 19-25 nucleotides in length, and is substantially complementary to at least part of an mRNA transcript of an Aha gene. The use of these dsRNAs enables the targeted degradation of mRNAs of genes that are implicated in replication and or maintenance of cancer cells in mammals, and/or in the degradation of misfolded Cystic Fibrosis Transmembrane Conductance Regulator (CFTR). Using cell-based and animal assays, very low dosages of these dsRNA can specifically and efficiently mediate RNAi, resulting in significant inhibition of expression of an Aha gene. Thus, the methods and compositions of the invention comprising these dsRNAs are useful for treating pathological processes mediated by Aha expression, e.g. protein misfolding, including cancer and/or cystic fibrosis, by targeting a gene involved in protein degradation.

The following detailed description discloses how to make and use the dsRNA and compositions containing dsRNA to inhibit the expression of an Aha gene, as well as compositions and methods for treating diseases and disorders caused by the expression of an Aha gene, such as cancer and/or cystic fibrosis. The pharmaceutical compositions of the invention comprise a dsRNA having an antisense strand comprising a region of complementarity which is less than 30 nucleotides in length, generally 19-25 nucleotides in length, and is substantially complementary to at least part of an RNA transcript of an Aha gene, together with a pharmaceutically acceptable carrier.

Accordingly, certain aspects of the invention provide pharmaceutical compositions comprising the dsRNA of the invention together with a pharmaceutically acceptable carrier, methods of using the compositions to inhibit expression of an Aha gene, and methods of using the pharmaceutical compositions to treat diseases caused by expression of an Aha gene.

One aspect of the present invention provides dsRNA molecules for inhibiting the expression of an Aha gene, e.g. an Aha1 gene, in a cell or mammal, wherein the dsRNA comprises an antisense strand comprising a region of complementarity which is complementary to at least a part of an mRNA formed in the expression of an Aha gene, e.g. an Aha1 gene, and wherein the region of complementarity is less than 30 nucleotides in length, generally 19-25 nucleotides in length. The dsRNA may be identical to one of the dsRNAs shown in Table 2, or it may effect cleavage of an mRNA encoding an Aha gene within the target sequence of one of the dsRNAs shown in Table 2.

TABLE 1

Homo sapiens Aha 1 mRNA Target Sequences (Sequence
Position Based on Coding Sequence of GenBank Ac-
cession No. NM_012111.1 (SEQ ID NO: 11; Ensembl
Gene Report No. ENSG00000100591))

1. mRNA Target Sequence Based on Aha Gene Sequence
AAATTGGTCCACGGATAAGCT:

| | |
|---|---|
| AAAUUGGUCCACGGAUAAGCU | (SEQ ID NO: 12) |

Position in gene sequence: 99
2. mRNA Target Sequence Based on Aha Gene Sequence
AAGCTGAAAACACTGTTCCTG:

| | |
|---|---|
| AAGCUGAAAACACUGUUCCUG | (SEQ ID NO: 13) |

Position in gene sequence: 115
3. mRNA Target Sequence Based on Aha Gene Sequence
AAAACACTGTTCCTGGCAGTG:

| | |
|---|---|
| AAAACACUGUUCCUGGCAGUG | (SEQ ID NO: 14) |

Position in gene sequence: 121
4. mRNA Target Sequence Based on Aha Gene Sequence
AAAATGAAGAAGGCAAGTGTG:

| | |
|---|---|
| AAAAUGAAGAAGGCAAGUGUG | (SEQ ID NO: 15) |

Position in gene sequence: 149
5. mRNA Target Sequence Based on Aha Gene Sequence
AATGAAGAAGGCAAGTGTGAG:

| | |
|---|---|
| AAUGAAGAAGGCAAGUGUGAG | (SEQ ID NO: 16) |

Position in gene sequence: 151
6. mRNA Target Sequence Based on Aha Gene Sequence
AAGAAGGCAAGTGTGAGGTGA:

| | |
|---|---|
| AAGAAGGCAAGUGUGAGGUGA | (SEQ ID NO: 17) |

Position in gene sequence: 155
7. mRNA Target Sequence Based on Aha Gene Sequence
AAGTGAGTAAGCTTGATGGAG:

| | |
|---|---|
| AAGUGAGUAAGCUUGAUGGAG | (SEQ ID NO: 18) |

Position in gene sequence: 179
8. mRNA Target Sequence Based on Aha Gene Sequence
AACAATCGCAAAGGGAAACTT:

| | |
|---|---|
| AACAAUCGCAAAGGGAAACUU | (SEQ ID NO: 19) |

Position in gene sequence: 211
9. mRNA Target Sequence Based on Aha Gene Sequence
AATCGCAAAGGGAAACTTATC:

| | |
|---|---|
| AAUCGCAAAGGGAAACUUAUC | (SEQ ID NO: 20) |

Position in gene sequence: 214
10. mRNA Target Sequence Based on Aha Gene
Sequence
AAAGGGAAACTTATCTTCTTT:

| | |
|---|---|
| AAAGGGAAACUUAUCUUCUUU | (SEQ ID NO: 21) |

Position in gene sequence: 220
11. mRNA Target Sequence Based on Aha Gene
Sequence
AAACTTATCTTCTTTTATGAA:

| | |
|---|---|
| AAACUUAUCUUCUUUUAUGAA | (SEQ ID NO: 22) |

Position in gene sequence: 226
12. mRNA Target Sequence Based on Aha Gene
Sequence
AATGGAGCGTCAAACTAAACT:

| | |
|---|---|
| AAUGGAGCGUCAAACUAAACU | (SEQ ID NO: 23) |

Position in gene sequence: 245
13. mRNA Target Sequence Based on Aha Gene
Sequence
AAACTAAACTGGACAGGTACT:

| | |
|---|---|
| AAACUAAACUGGACAGGUACU | (SEQ ID NO: 24) |

Position in gene sequence: 256
14. mRNA Target Sequence Based on Aha Gene
Sequence
AAACTGGACAGGTACTTCTAA:

| | |
|---|---|
| AAACUGGACAGGUACUUCUAA | (SEQ ID NO: 25) |

Position in gene sequence: 261
15. mRNA Target Sequence Based on Aha Gene
Sequence
AAGTCAGGAGTACAATACAAA:

| | |
|---|---|
| AAGUCAGGAGUACAAUACAAA | (SEQ ID NO: 26) |

Position in gene sequence: 280
16. mRNA Target Sequence Based on Aha Gene
Sequence
AATACAAAGGACATGTGGAGA:

| | |
|---|---|
| AAUACAAAGGACAUGUGGAGA | (SEQ ID NO: 27) |

Position in gene sequence: 293
17. mRNA Target Sequence Based on Aha Gene
Sequence
AATTTGTCTGATGAAAACAGC:

| | |
|---|---|
| AAUUUGUCUGAUGAAAACAGC | (SEQ ID NO: 28) |

Position in gene sequence: 319
18. mRNA Target Sequence Based on Aha Gene
Sequence
AAAACAGCGTGGATGAAGTGG:

| | |
|---|---|
| AAAACAGCGUGGAUGAAGUGG | (SEQ ID NO: 29) |

Position in gene sequence: 332
19. mRNA Target Sequence Based on Aha Gene
Sequence
AAGTGGAGATTAGTGTGAGCC:

| | |
|---|---|
| AAGUGGAGAUUAGUGUGAGCC | (SEQ ID NO: 30) |

Position in gene sequence: 347
20. mRNA Target Sequence Based on Aha Gene
Sequence
AAAGATGAGCCTGACACAAAT:

| | |
|---|---|
| AAAGAUGAGCCUGACACAAAU | (SEQ ID NO: 31) |

Position in gene sequence: 373
21. mRNA Target Sequence Based on Aha Gene
Sequence
AAATCTCGTGGCCTTAATGAA:

| | |
|---|---|
| AAAUCUCGUGGCCUUAAUGAA | (SEQ ID NO: 32) |

Position in gene sequence: 390
22. mRNA Target Sequence Based on Aha Gene
Sequence
AATGAAGGAAGAAGGGGTGAA:

| | |
|---|---|
| AAUGAAGGAAGAAGGGGUGAA | (SEQ ID NO: 33) |

Position in gene sequence: 405
23. mRNA Target Sequence Based on Aha Gene
Sequence
AAGGAAGAAGGGGTGAAACTT:

| | |
|---|---|
| AAGGAAGAAGGGGUGAAACUU | (SEQ ID NO: 34) |

Position in gene sequence: 409
24. mRNA Target Sequence Based on Aha Gene
Sequence
AAGAAGGGGTGAAACTTCTAA:

| | |
|---|---|
| AAGAAGGGGUGAAACUUCUAA | (SEQ ID NO: 35) |

Position in gene sequence: 413
25. mRNA Target Sequence Based on Aha Gene
Sequence
AAGGGGTGAAACTTCTAAGAG:

| | |
|---|---|
| AAGGGGUGAAACUUCUAAGAG | (SEQ ID NO: 36) |

Position in gene sequence: 416
26. mRNA Target Sequence Based on Aha Gene
Sequence
AAACTTCTAAGAGAAGCAATG:

| | |
|---|---|
| AAACUUCUAAGAGAAGCAAUG | (SEQ ID NO: 37) |

Position in gene sequence: 424
27. mRNA Target Sequence Based on Aha Gene
Sequence
AAGAGAAGCAATGGGAATTTA:

| | |
|---|---|
| AAGAGAAGCAAUGGGAAUUUA | (SEQ ID NO: 38) |

Position in gene sequence: 432
28. mRNA Target Sequence Based on Aha Gene
Sequence
AAGCAATGGGAATTTACATCA:

| | |
|---|---|
| AAGCAAUGGGAAUUUACAUCA | (SEQ ID NO: 39) |

Position in gene sequence: 437
29. mRNA Target Sequence Based on Aha Gene
Sequence
AATGGGAATTTACATCAGCAC:

| | |
|---|---|
| AAUGGGAAUUUACAUCAGCAC | (SEQ ID NO: 40) |

Position in gene sequence: 441
30. mRNA Target Sequence Based on Aha Gene
Sequence
AATTTACATCAGCACCCTCAA:

| | |
|---|---|
| AAUUUACAUCAGCACCCUCAA | (SEQ ID NO: 41) |

Position in gene sequence: 447
31. mRNA Target Sequence Based on Aha Gene
Sequence
AATGAATGGAGAGTCAGTAGA:

| | |
|---|---|
| AAUGAAUGGAGAGUCAGUAGA | (SEQ ID NO: 42) |

Position in gene sequence: 501
32. mRNA Target Sequence Based on Aha Gene
Sequence
AATGGAGAGTCAGTAGACCCA:

| | |
|---|---|
| AAUGGAGAGUCAGUAGACCCA | (SEQ ID NO: 43) |

Position in gene sequence: 505
33. mRNA Target Sequence Based on Aha Gene
Sequence
AAGCCTGCTCCTTCAAAAACC:

| | |
|---|---|
| AAGCCUGCUCCUUCAAAAACC | (SEQ ID NO: 44) |

Position in gene sequence: 565
34. mRNA Target Sequence Based on Aha Gene
Sequence
AAAATCCCCACTTGTAAGATC:

| | |
|---|---|
| AAAAUCCCCACUUGUAAGAUC | (SEQ ID NO: 45) |

Position in gene sequence: 607
35. mRNA Target Sequence Based on Aha Gene
Sequence
AATCCCCACTTGTAAGATCAC:

| | |
|---|---|
| AAUCCCCACUUGUAAGAUCAC | (SEQ ID NO: 46) |

Position in gene sequence: 609
36. mRNA Target Sequence Based on Aha Gene
Sequence
AAGATCACTCTTAAGGAAACC:

| | |
|---|---|
| AAGAUCACUCUUAAGGAAACC | (SEQ ID NO: 47) |

Position in gene sequence: 622
37. mRNA Target Sequence Based on Aha Gene
Sequence
AAGGAAACCTTCCTGACGTCA:

| | |
|---|---|
| AAGGAAACCUUCCUGACGUCA | (SEQ ID NO: 48) |

Position in gene sequence: 634
38. mRNA Target Sequence Based on Aha Gene
Sequence
AACATTAGAAGCAGACAGAGG:

| | |
|---|---|
| AACAUUAGAAGCAGACAGAGG | (SEQ ID NO: 49) |

Position in gene sequence: 720
39. mRNA Target Sequence Based on Aha Gene
Sequence
AAGCAGACAGAGGTGGAAAGT:

| | |
|---|---|
| AAGCAGACAGAGGUGGAAAGU | (SEQ ID NO: 50) |

Position in gene sequence: 728
40. mRNA Target Sequence Based on Aha Gene
Sequence
AAAGTTCCACATGGTAGATGG:

| | |
|---|---|
| AAAGUUCCACAUGGUAGAUGG | (SEQ ID NO: 51) |

Position in gene sequence: 744
41. mRNA Target Sequence Based on Aha Gene
Sequence
AACGTCTCTGGGGAATTTACT:

| | |
|---|---|
| AACGUCUCUGGGGAAUUUACU | (SEQ ID NO: 52) |

Position in gene sequence: 766
42. mRNA Target Sequence Based on Aha Gene
Sequence
AATTTACTGATCTGGTCCCTG:

| | |
|---|---|
| AAUUUACUGAUCUGGUCCCUG | (SEQ ID NO: 53) |

Position in gene sequence: 779
43. mRNA Target Sequence Based on Aha Gene
Sequence
AAACATATTGTGATGAAGTGG:

| | |
|---|---|
| AAACAUAUUGUGAUGAAGUGG | (SEQ ID NO: 54) |

Position in gene sequence: 802
44. mRNA Target Sequence Based on Aha Gene
Sequence
AAGTGGAGGTTTAAATCTTGG:

| | |
|---|---|
| AAGUGGAGGUUUAAAUCUUGG | (SEQ ID NO: 55) |

Position in gene sequence: 817
45. mRNA Target Sequence Based on Aha Gene
Sequence
AAACAGACCTTTGGCTATGGC:

| | |
|---|---|
| AAACAGACCUUUGGCUAUGGC | (SEQ ID NO: 56) |

Position in gene sequence: 982

TABLE 2

dsRNA agents for the down-regulation of Homo
sapiens Functional Aha Protein Expression

| | |
|---|---|
| 1. | dsRNA based on Aha Gene Target Sequence 1 |

| | |
|---|---|
| Sense strand dsRNA: | |
| AUUGGUCCACGGAUAAGCU | (SEQ ID NO: 57) |
| Antisense strand dsRNA: | |
| AGCUUAUCCGUGGACCAAU | (SEQ ID NO: 58) |

| | |
|---|---|
| 2. | dsRNA based on Aha Gene Target Sequence 2 |

| | |
|---|---|
| Sense strand dsRNA: | |
| GCUGAAAACACUGUUCCUG | (SEQ ID NO: 59) |
| Antisense strand dsRNA: | |
| CAGGAACAGUGUUUUCAGC | (SEQ ID NO: 60) |

| | |
|---|---|
| 3. | dsRNA based on Aha Gene Target Sequence 3 |

| | |
|---|---|
| Sense strand dsRNA: | |
| AACACUGUUCCUGGCAGUG | (SEQ ID NO: 61) |
| Antisense strand dsRNA: | |
| CACUGCCAGGAACAGUGUU | (SEQ ID NO: 62) |

| | |
|---|---|
| 4. | dsRNA based on Aha Gene Target Sequence 4 |

| | |
|---|---|
| Sense strand dsRNA: | |
| AAUGAAGAAGGCAAGUGUG | (SEQ ID NO: 63) |
| Antisense strand dsRNA: | |
| CACACUUGCCUUCUUCAUU | (SEQ ID NO: 64) |

| | |
|---|---|
| 5. | dsRNA based on Aha Gene Target Sequence 5 |

| | |
|---|---|
| Sense strand dsRNA: | |
| UGAAGAAGGCAAGUGUGAG | (SEQ ID NO: 65) |
| Antisense strand dsRNA: | |
| CUCACACUUGCCUUCUUCA | (SEQ ID NO: 66) |

| | |
|---|---|
| 6. | dsRNA based on Aha Gene Target Sequence 6 |

| | |
|---|---|
| Sense strand dsRNA: | |
| GAAGGCAAGUGUGAGGUGA | (SEQ ID NO: 67) |
| Antisense strand dsRNA: | |
| UCACCUCACACUUGCCUUC | (SEQ ID NO: 68) |

| | |
|---|---|
| 7. | dsRNA based on Aha Gene Target Sequence 7 |

| | |
|---|---|
| Sense strand dsRNA: | |
| GUGAGUAAGCUUGAUGGAG | (SEQ ID NO: 69) |
| Antisense strand dsRNA: | |
| CUCCAUCAAGCUUACUCAC | (SEQ ID NO: 70) |

| | |
|---|---|
| 8. | dsRNA based on Aha Gene Target Sequence 8 |

| | |
|---|---|
| Sense strand dsRNA: | |
| CAAUCGCAAAGGGAAACUU | (SEQ ID NO: 71) |
| Antisense strand dsRNA: | |
| AAGUUUCCCUUUGCGAUUG | (SEQ ID NO: 72) |

| | |
|---|---|
| 9. | dsRNA based on Aha Gene Target Sequence 9 |

| | |
|---|---|
| Sense strand dsRNA: | |
| UCGCAAAGGGAAACUUAUC | (SEQ ID NO: 73) |
| Antisense strand dsRNA: | |
| GAUAAGUUUCCCUUUGCGA | (SEQ ID NO: 74) |

| | |
|---|---|
| 10. | dsRNA based on Aha Gene Target Sequence 10 |

| | |
|---|---|
| Sense strand dsRNA: | |
| AGGGAAACUUAUCUUCUUU | (SEQ ID NO: 75) |
| Antisense strand dsRNA: | |
| AAAGAAGAUAAGUUUCCCU | (SEQ ID NO: 76) |

| | |
|---|---|
| 11. | dsRNA based on Aha Gene Target Sequence 11 |

| | |
|---|---|
| Sense strand dsRNA: | |
| ACUUAUCUUCUUUUAUGAA | (SEQ ID NO: 77) |
| Antisense strand dsRNA: | |
| UUCAUAAAAGAAGAUAAGU | (SEQ ID NO: 78) |

| | |
|---|---|
| 12. | dsRNA based on Aha Gene Target Sequence 12 |

| | |
|---|---|
| Sense strand dsRNA: | |
| UGGAGCGUCAAACUAAACU | (SEQ ID NO: 79) |
| Antisense strand dsRNA: | |
| AGUUUAGUUUGACGCUCCA | (SEQ ID NO: 80) |

| | |
|---|---|
| 13. | dsRNA based on Aha Gene Target Sequence 13 |

| | |
|---|---|
| Sense strand dsRNA: | |
| ACUAAACUGGACAGGUACU | (SEQ ID NO: 81) |
| Antisense strand dsRNA: | |
| AGUACCUGUCCAGUUUAGU | (SEQ ID NO: 82) |

| | |
|---|---|
| 14. | dsRNA based on Aha Gene Target Sequence 14 |

| | |
|---|---|
| Sense strand dsRNA: | |
| ACUGGACAGGUACUUCUAA | (SEQ ID NO: 83) |
| Antisense strand dsRNA: | |
| UUAGAAGUACCUGUCCAGU | (SEQ ID NO: 84) |

| | |
|---|---|
| 15. | dsRNA based on Aha Gene Target Sequence 15 |

| | |
|---|---|
| Sense strand dsRNA: | |
| GUCAGGAGUACAAUACAAA | (SEQ ID NO: 85) |
| Antisense strand dsRNA: | |
| UUUGUAUUGUACUCCUGAC | (SEQ ID NO: 86) |

| | |
|---|---|
| 16. | dsRNA based on Aha Gene Target Sequence 16 |

| | |
|---|---|
| Sense strand dsRNA: | |
| UACAAAGGACAUGUGGAGA | (SEQ ID NO: 87) |
| Antisense strand dsRNA: | |
| UCUCCACAUGUCCUUUGUA | (SEQ ID NO: 88) |

| | |
|---|---|
| 17. | dsRNA based on Aha Gene Target Sequence 17 |

| | |
|---|---|
| Sense strand dsRNA: | |
| UUUGUCUGAUGAAAACAGC | (SEQ ID NO: 89) |
| Antisense strand dsRNA: | |
| GCUGUUUUCAUCAGACAAA | (SEQ ID NO: 90) |

| | |
|---|---|
| 18. | dsRNA based on Aha Gene Target Sequence 18 |

| | |
|---|---|
| Sense strand dsRNA: | |
| AACAGCGUGGAUGAAGUGG | (SEQ ID NO: 91) |
| Antisense strand dsRNA: | |
| CCACUUCAUCCACGCUGUU | (SEQ ID NO: 92) |

| | |
|---|---|
| 19. | dsRNA based on Aha Gene Target Sequence 19 |

| | |
|---|---|
| Sense strand dsRNA: | |
| GUGGAGAUUAGUGUGAGCC | (SEQ ID NO: 93) |
| Antisense strand dsRNA: | |
| GGCUCACACUAAUCUCCAC | (SEQ ID NO: 94) |

| | |
|---|---|
| 20. | dsRNA based on Aha Gene Target Sequence 20 |

| | |
|---|---|
| Sense strand dsRNA: | |
| AGAUGAGCCUGACACAAAU | (SEQ ID NO: 95) |
| Antisense strand dsRNA: | |
| AUUUGUGUCAGGCUCAUCU | (SEQ ID NO: 96) |

| | |
|---|---|
| 21. | dsRNA based on Aha Gene Target Sequence 21 |

| | |
|---|---|
| Sense strand dsRNA: | |
| AUCUCGUGGCCUUAAUGAA | (SEQ ID NO: 97) |
| Antisense strand dsRNA: | |
| UUCAUUAAGGCCACGAGAU | (SEQ ID NO: 98) |

| | |
|---|---|
| 22. | dsRNA based on Aha Gene Target Sequence 22 |

| | |
|---|---|
| Sense strand dsRNA: | |
| UGAAGGAAGAAGGGGUGAA | (SEQ ID NO: 99) |
| Antisense strand dsRNA: | |
| UUCACCCCUUCUUCCUUCA | (SEQ ID NO: 100) |

| | |
|---|---|
| 23. | dsRNA based on Aha Gene Target Sequence 23 |

| | |
|---|---|
| Sense strand dsRNA: | |
| GGAAGAAGGGGUGAAACUU | (SEQ ID NO: 101) |
| Antisense strand dsRNA: | |
| AAGUUUCACCCCUUCUUCC | (SEQ ID NO: 102) |

| | |
|---|---|
| 24. | dsRNA based on Aha Gene Target Sequence 24 |

| | |
|---|---|
| Sense strand dsRNA: | |
| GAAGGGGUGAAACUUCUAA | (SEQ ID NO: 103) |
| Antisense strand dsRNA: | |
| UUAGAAGUUUCACCCCUUC | (SEQ ID NO: 104) |

| | |
|---|---|
| 25. | dsRNA based on Aha Gene Target Sequence 25 |

| | |
|---|---|
| Sense strand dsRNA: | |
| GGGGUGAAACUUCUAAGAG | (SEQ ID NO: 105) |
| Antisense strand dsRNA: | |
| CUCUUAGAAGUUUCACCCC | (SEQ ID NO: 106) |

| | |
|---|---|
| 26. | dsRNA based on Aha Gene Target Sequence 26 |

| | |
|---|---|
| Sense strand dsRNA: | |
| ACUUCUAAGAGAAGCAAUG | (SEQ ID NO: 107) |
| Antisense strand dsRNA: | |
| CAUUGCUUCUCUUAGAAGU | (SEQ ID NO: 108) |

| | |
|---|---|
| 27. | dsRNA based on Aha Gene Target Sequence 27 |

| | |
|---|---|
| Sense strand dsRNA: | |
| GAGAAGCAAUGGGAAUUUA | (SEQ ID NO: 109) |
| Antisense strand dsRNA: | |
| UAAAUUCCCAUUGCUUCUC | (SEQ ID NO: 110) |

| | |
|---|---|
| 28. | dsRNA based on Aha Gene Target Sequence 28 |

| | |
|---|---|
| Sense strand dsRNA: | |
| GCAAUGGGAAUUUACAUCA | (SEQ ID NO: 111) |
| Antisense strand dsRNA: | |
| UGAUGUAAAUUCCCAUUGC | (SEQ ID NO: 112) |

| | |
|---|---|
| 29. | dsRNA based on Aha Gene Target Sequence 29 |

| | |
|---|---|
| Sense strand dsRNA: | |
| UGGGAAUUUACAUCAGCAC | (SEQ ID NO: 113) |
| Antisense strand dsRNA: | |
| GUGCUGAUGUAAAUUCCCA | (SEQ ID NO: 114) |

| | |
|---|---|
| 30. | dsRNA based on Aha Gene Target Sequence 30 |

| | |
|---|---|
| Sense strand dsRNA: | |
| UUUACAUCAGCACCCUCAA | (SEQ ID NO: 115) |
| Antisense strand dsRNA: | |
| UUGAGGGUGCUGAUGUAAA | (SEQ ID NO: 116) |

| | |
|---|---|
| 31. | dsRNA based on Aha Gene Target Sequence 31 |

| | |
|---|---|
| Sense strand dsRNA: | |
| UGAAUGGAGAGUCAGUAGA | (SEQ ID NO: 117) |
| Antisense strand dsRNA: | |
| UCUACUGACUCUCCAUUCA | (SEQ ID NO: 118) |

| | |
|---|---|
| 32. | dsRNA based on Aha Gene Target Sequence 32 |

| | |
|---|---|
| Sense strand dsRNA: | |
| UGGAGAGUCAGUAGACCCA | (SEQ ID NO: 119) |
| Antisense strand dsRNA: | |
| UGGGUCUACUGACUCUCCA | (SEQ ID NO: 120) |

| | |
|---|---|
| 33. | dsRNA based on Aha Gene Target Sequence 33 |

| | |
|---|---|
| Sense strand dsRNA: | |
| GCCUGCUCCUUCAAAAACC | (SEQ ID NO: 121) |
| Antisense strand dsRNA: | |
| GGUUUUUGAAGGAGCAGGC | (SEQ ID NO: 122) |

| | |
|---|---|
| 34. | dsRNA based on Aha Gene Target Sequence 34 |

| | |
|---|---|
| Sense strand dsRNA: | |
| AAUCCCCACUUGUAAGAUC | (SEQ ID NO: 123) |
| Antisense strand dsRNA: | |
| GAUCUUACAAGUGGGGAUU | (SEQ ID NO: 124) |

| | |
|---|---|
| 35. | dsRNA based on Aha Gene Target Sequence 35 |

| | |
|---|---|
| Sense strand dsRNA: | |
| UCCCCACUUGUAAGAUCAC | (SEQ ID NO: 125) |
| Antisense strand dsRNA: | |
| GUGAUCUUACAAGUGGGGA | (SEQ ID NO: 126) |

| | |
|---|---|
| 36. | dsRNA based on Aha Gene Target Sequence 36 |

| | |
|---|---|
| Sense strand dsRNA: | |
| GAUCACUCUUAAGGAAACC | (SEQ ID NO: 127) |
| Antisense strand dsRNA: | |
| GGUUUCCUUAAGAGUGAUC | (SEQ ID NO: 128) |

| | |
|---|---|
| 37. | dsRNA based on Aha Gene Target Sequence 37 |

| | |
|---|---|
| Sense strand dsRNA: | |
| GGAAACCUUCCUGACGUCA | (SEQ ID NO: 129) |
| Antisense strand dsRNA: | |
| UGACGUCAGGAAGGUUUCC | (SEQ ID NO: 130) |

| | |
|---|---|
| 38. | dsRNA based on Aha Gene Target Sequence 38 |

| | |
|---|---|
| Sense strand dsRNA: | |
| CAUUAGAAGCAGACAGAGG | (SEQ ID NO: 131) |
| Antisense strand dsRNA: | |
| CCUCUGUCUGCUUCUAAUG | (SEQ ID NO: 132) |

| | |
|---|---|
| 39. | dsRNA based on Aha Gene Target Sequence 39 |

| | |
|---|---|
| Sense strand dsRNA: | |
| GCAGACAGAGGUGGAAAGU | (SEQ ID NO: 133) |
| Antisense strand dsRNA: | |
| ACUUUCCACCUCUGUCUGC | (SEQ ID NO: 134) |

| | |
|---|---|
| 40. | dsRNA based on Aha Gene Target Sequence 40 |

| | |
|---|---|
| Sense strand dsRNA: | |
| AGUUCCACAUGGUAGAUGG | (SEQ ID NO: 135) |
| Antisense strand dsRNA: | |
| CCAUCUACCAUGUGGAACU | (SEQ ID NO: 136) |

| | |
|---|---|
| 41. | dsRNA based on Aha Gene Target Sequence 41 |

| | |
|---|---|
| Sense strand dsRNA: | |
| CGUCUCUGGGGAAUUUACU | (SEQ ID NO: 137) |
| Antisense strand dsRNA: | |
| AGUAAAUUCCCCAGAGACG | (SEQ ID NO: 138) |

| | |
|---|---|
| 42. | dsRNA based on Aha Gene Target Sequence 42 |

| | |
|---|---|
| Sense strand dsRNA: | |
| UUUACUGAUCUGGUCCCUG | (SEQ ID NO: 139) |
| Antisense strand dsRNA: | |
| CAGGGACCAGAUCAGUAAA | (SEQ ID NO: 140) |

| | |
|---|---|
| 43. | dsRNA based on Aha Gene Target Sequence 43 |

| | |
|---|---|
| Sense strand dsRNA: | |
| ACAUAUUGUGAUGAAGUGG | (SEQ ID NO: 141) |
| Antisense strand dsRNA: | |
| CCACUUCAUCACAAUAUGU | (SEQ ID NO: 142) |

| | |
|---|---|
| 44. | dsRNA based on Aha Gene Target Sequence 44 |

| | |
|---|---|
| Sense strand dsRNA: | |
| GUGGAGGUUUAAAUCUUGG | (SEQ ID NO: 143) |
| Antisense strand dsRNA: | |
| CCAAGAUUUAAACCUCCAC | (SEQ ID NO: 144) |

| | |
|---|---|
| 45. | dsRNA based on Aha Gene Target Sequence 45 |

| | |
|---|---|
| Sense strand dsRNA: | |
| ACAGACCUUUGGCUAUGGC | (SEQ ID NO: 145) |
| Antisense strand dsRNA: | |
| GCCAUAGCCAAAGGUCUGU | (SEQ ID NO: 146) |

TABLE 3

Primers for Vector Transcribing shRNA for the
down-regulation of Homo sapiens Functional Aha
Protein Expression

| | |
|---|---|
| 1. | Primer based on Aha Gene Target Sequence 1 |
| | (SEQ ID NO: 12) |
| | Sense strand: |

(SEQ ID NO: 147)

GATCCATTGGTCCACGGATAAGCTTTCAAGAGAAGCTTATCCGTG
GACCAATTTTTTTGGAAA
Antisense strand:

(SEQ ID NO: 148)

AGCTTTTCCAAAAAAATTGGTCCACGGATAAGCTTCTCTTGAAAG
CTTATCCGTGGACCAATG

| | |
|---|---|
| 2. | Primer based on Aha Gene Target Sequence 7 |
| | (SEQ ID NO: 18) |
| | Sense strand: |

(SEQ ID NO: 149)

GATCCGTGAGTAAGCTTGATGGAGTTCAAGAGACTCCATCAAGC
TTACTCACTTTTTTGGAAA
Antisense strand:

(SEQ ID NO: 150)

AGCTTTTCCAAAAAAGTGAGTAAGCTTGATGGAGTCTCTTGAACT
CCATCAAGCTTACTCACG

| | |
|---|---|
| 3. | Primer based on Aha Gene Target Sequence 13 |
| | (SEQ ID NO: 24) |
| | Sense strand: |

(SEQ ID NO: 151)

GATCCACTAAACTGGACAGGTACTTTCAAGAGAAGTACCTGTCCA
GTTTAGTTTTTTTGGAAA
Antisense strand:

(SEQ ID NO: 152)

AGCTTTTCCAAAAAAACTAAACTGGACAGGTACTTCTCTTGAAAG
TACCTGTCCAGTTTAGTG

TABLE 4
shRNA Sequences Transcribed by Encoding Vector

(SEQ ID NO: 153)

1.
GAUCCAUUGGUCCACGGAUAAGCUUUCAAGAGAAGCUUAUCCGUGGACCA
AUUUUUUUGGAAA

(SEQ ID NO: 154)

2.
GAUCCGUGAGUAAGCUUGAUGGAGUUCAAGAGACUCCAUCAAGCUUACUC
ACUUUUUUGGAAA

(SEQ ID NO: 155)

3.
GAUCCACUAAACUGGACAGGUACUUUCAAGAGAAGUACCUGUCCAGUUUA
GUUUUUUUGGAAA

In various aspects of the present invention, the dsRNA can have at least 5, at least 10, at least 15, at least 18, or at least 20 contiguous nucleotides per strand in common with at least one strand, and in various aspects both strands, of one of the dsRNAs shown in Table 2. Alternative dsRNAs that target elsewhere in the target sequence of one of the dsRNAs provided in Table 2 can readily be determined using the target sequence and the flanking Aha1 sequence.

The dsRNA comprises two RNA strands that are complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of an Aha gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). For example, deoxyribonucleotide sequence “tt” or ribonucleotide sequence “UU” can be connected to the 3′-end of both sense and antisense strands to form overhangs. The dsRNA can be synthesized by standard methods known in the art as further discussed below, e.g., by use of an automated DNA synthesizer, such as are commercially available from, for example, Biosearch, Applied Biosystems, Inc. In one aspect of the present invention, an Aha gene can be a human Aha1 gene.

In various aspects, the dsRNA comprises at least two sequences selected from this group, wherein one of the at least two sequences is complementary to another of the at least two sequences, and one of the at least two sequences is substantially complementary to a sequence of an mRNA generated in the expression of an Aha gene, e.g. an Aha1 gene.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well.

The dsRNA of the invention can contain one to three mismatches to the target sequence. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity, and preferably from the 5′-end. For example, for a 23 nucleotide dsRNA strand which is complementary to a region of an Aha gene, the dsRNA generally does not contain any mismatch within the central 13 nucleotides. In another aspect, the antisense strand of the dsRNA does not contain any mismatch in the region from positions 1, or 2, to positions 9, or 10, of the antisense strand (counting 5′-3′). The methods described within the invention can be used to determine whether a dsRNA containing a mismatch to a target sequence is effective in inhibiting the expression of an Aha gene. Consideration of the efficacy of dsRNAs with mismatches in inhibiting expression of an Aha gene is important, especially if the particular region of complementarity in an Aha gene is known to have polymorphic sequence variation within the population.

In one aspect, at least one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. Generally, the antisense strand of the dsRNA has a nucleotide overhang at the 3′-end, and the 5′-end is blunt. In another aspect, one or more of the nucleotides in the overhang is replaced with a nucleoside thiophosphate.

Vector Encoded RNAi Agents

The dsRNA of the invention can also be expressed from recombinant viral vectors intracellularly in vivo. The recombinant viral vectors of the invention comprise sequences encoding the dsRNA of the invention and any suitable promoter for expressing the dsRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the dsRNA in a particular tissue or in a particular intracellular environment. The use of recombinant viral vectors to deliver dsRNA of the invention to cells in vivo is discussed in more detail below.

dsRNA of the invention can be expressed from a recombinant viral vector either as two separate, complementary RNA molecules, or as a single RNA molecule with two complementary regions.

Those of skill in the art will recognize that any viral vector capable of accepting the coding sequences for the dsRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference. For example, the dsRNA of the invention is expressed as two separate, complementary single-stranded RNA molecules from a recombinant AAV vector comprising, for example, either the U6 or H1 RNA promoters, or the cytomegalovirus (CMV) promoter. A suitable AV vector for expressing the dsRNA of the invention, a method for constructing the recombinant AV vector, and a method for delivering the vector into target cells, are described in Xia H et al. (2002), Nat. Biotech. 20: 1006-1010. Suitable AAV vectors for expressing the dsRNA of the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Non-dsRNA Agents

Antibodies can be used to decrease levels of functional Aha1 and/or other related molecules with similar function. For example, antibodies can decrease levels of functional Aha1 by specifically binding to functional Aha1, the Hsp90 ATPase binding site for functional Aha1, and/or the functional Aha1-Hsp90 ATPase complex. Antibodies within the scope of the invention include, for example, polyclonal antibodies, monoclonal antibodies, antibody fragments, and antibody-based fusion molecules. Engineering, production, screening, purification, fragmentation, and therapeutic use of antibodies are well known in the art (see generally, Carter (2006) Nat Rev Immunol. 6(5), 343-357; Coligan (2005) Short Protocols in Immunology, John Wiley & Sons, ISBN 0471715786); Teillaud (2005) Expert Opin Biol Ther. 5(Supp. 1) S15-27; Subramanian, ed. (2004) Antibodies: Volume 1: Production and Purification, Springer, ISBN 0306482452; Brent et al., ed. (2003) Current Protocols in Molecular Biology, John Wiley & Sons Inc, ISBN 047150338X; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929). Various types of antibodies specific for functional Aha1 can also be obtained from a variety of commercial sources. The terminal half-life of antibodies in plasma can be tuned over a wide range, for example several minutes to several weeks, to fit clinical goals for treating protein misfolding diseases (see e.g., Carter et al. (2006) Nat Rev Immunol. 6(5), 343-357, 353). Chimeric, humanized, and fully human MAbs can effectively overcome potential limitations on the use of antibodies derived from non-human sources to treat protein misfolding diseases, thus providing decreased immunogenicity with optimized effector functions (see e.g., Teillaud (2005) Expert Opin. Biol. Ther. 5(1), S15-S27; Tomizuka et al. (2000) Proc. Nat. Acad. Sci. USA 97, 722-727; Carter et al. (2006) Nat Rev Immunol. 6(5), 343-357, 346-347). Antibodies can be altered or selected so as to achieve efficient antibody internalization. As such, the antibodies can more effectively interact with target intracellular molecules, such as functional Aha1 and/or related molecules with similar functions, or complexes including such. Further, antibody-drug conjugates can increase the efficiency of antibody internalization. Efficient antibody internalization can be desirable for delivering functional Aha1 specific antibodies to the intracellular environment so as to salvage defective folding and transit of proteins characterized by suboptimal folding energetics. Conjugation of antibodies to a variety of agents that can facilitate cellular internalization of antibodies is known in the art (see generally Wu et al. (2005) Nat. Biotechnol. 23(9), 1137-1146; McCarron et al. (2005) Mol Interv 5(6), 368-380; Niemeyer (2004) Bioconjugation Protocols, Strategies and Methods, Humana Press, ISBN 1588290980; Hermanson (1996) Bioconjugate Techniques, Academic Press, ISBN 0123423368).

Small organic molecules that interact specifically with heat shock protein co-chaperones, such as the Hsp90 co-chaperone Aha1, can be used to decrease the levels of functional Aha1 and/or other related molecules with similar functions. Identification of a pharmaceutical or small molecule inhibitor of functional Aha1 can be readily accomplished through standard high-throughput screening methods. Furthermore, standard medical chemistry approaches can be applied to these agents to enhance or modify their activity so as to yield additional agents.

Purified aptamers that specifically recognize and bind to functional Aha1 (or other related molecules with similar function) nucleotides or proteins can be used to decrease the level of functional Aha1 (and/or other related molecules with similar functions). Aptamers are nucleic acids or peptide molecules selected from a large random sequence pool to bind to specific target molecule. The small size of aptamers makes them easier to synthesize and chemically modify and enables them to access epitopes that otherwise might be blocked or hidden. And aptamers are generally nontoxic and weak antigens because of their close resemblance to endogenous molecules. Generation, selection, and delivery of aptamers is within the skill of the art (see e.g., Lee et al. (2006) Curr Opin Chem. Biol. 10, 1-8; Yan et al. (2005) Front Biosci 10, 1802-1827; Hoppe-Seyler and Butz (2000) J Mol. Med. 78(8), 426-430). Negative selection procedures can yield aptamers that can finely discriminate between molecular variants. For example, negative selection procedures can yield aptamers that can discriminate between Hsp90/ADP and Hsp90/ATP; or can discriminate between functional Aha1, Hsp90 ATPase, and the functional Aha1-Hsp90 ATPase binding complex. Aptamers can also be used to temporally and spatially regulate protein function (e.g., functional Aha1 function) in cells and organisms. For example, the ligand-regulated peptide (LiRP) system provides a general method where the binding activity of intracellular peptides is controlled by a peptide aptamer in turn regulated by a cell-permeable small molecule (see e.g., Binkowski (2005) Chem & Biol. 12(7), 847-55). Using LiRP or a similar delivery system, the binding activity of functional Aha1 could be controlled by a cell-permeable small molecule that interacts with the introduced intracellular functional Aha1-specific protein aptamer. Thus, aptamers can provide an effective means to decrease functional Aha1 levels by, for example, directly binding the functional Aha1 mRNA, functional Aha1 expressed protein, the Hsp90 ATPase binding site for functional Aha1, and/or the functional Aha1-Hsp90 ATPase complex.

Purified antisense nucleic acids that specifically recognize and bind to ribonucleotides encoding functional Aha1 (and/or other related molecules with similar function) can be used to decrease the levels of functional Aha1 (and/or other related molecules with similar functions). Antisense nucleic acid molecules within the invention are those that specifically hybridize (e.g., bind) under cellular conditions to cellular mRNA and/or genomic DNA encoding, for example functional Aha1 protein, in a manner that inhibits expression of that protein, e.g., by inhibiting transcription and/or translation. Antisense molecules, effective for decreasing functional Aha1 levels, can be designed, produced, and administered by methods commonly known to the art (see e.g., Chan et al. (2006) Clinical and Experimental Pharmacology and Physiology 33(5-6), 533-540).

Ribozyme molecules designed to catalytically cleave target mRNA transcripts can also be used to decrease levels of functional Aha1 and/or related molecules with similar activity. Ribozyme molecules specific for functional Aha1 can be designed, produced, and administered by methods commonly known to the art (see e.g., Fanning and Symonds (2006) Handbook Experimental Pharmacology 173, 289-303G, reviewing therapeutic use of hammerhead ribozymes and small hairpin RNA). Triplex-forming oligonucleotides can also be used to decrease levels of functional Aha1 and/or related molecules with similar activity (see generally, Rogers et al. (2005) Current Medicinal Chemistry 5(4), 319-326).

Administration

Agents for use in the methods described herein can be delivered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

The agents described herein can be formulated by any conventional manner using one or more pharmaceutically acceptable carriers and/or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of the agent, preferably in purified form, together with a suitable amount of carrier so as to provide the form for proper administration to the subject. The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents of the present invention and/or together with other biologically active or biologically inert agents. Such biologically active or inert agents may be in fluid or mechanical communication with the agent(s) or attached to the agent(s) by ionic, covalent, Van der Waals, hydrophobic, hydrophillic or other physical forces.

When used in the methods of the invention, a therapeutically effective amount of one of the agents described herein can be employed in pure form or, where such forms exist, in pharmaceutically acceptable salt form and with or without a pharmaceutically acceptable excipient. For example, the agents of the invention can be administered, at a reasonable benefit/risk ratio applicable to any medical treatment, in a sufficient amount sufficient to rescue intracellular and/or extracellular trafficking of a protein characterized by suboptimal folding energetics and/or at least partially restore channel functions in a subject.

Toxicity and therapeutic efficacy of such agents can be determined by standard pharmaceutical procedures in cell cultures and/or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where large therapeutic indices are preferred.

The amount of an agent that may be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses. Agent administration can occur as a single event or over a time course of treatment. For example, an agent can be administered daily, weekly, bi-weekly, or monthly. For some conditions, treatment could extend from several weeks to several months or even a year or more.

The specific therapeutically effective dose level for any particular subject will depend upon a variety of factors including the disorder being treated and the severity of the disorder; activity of the specific agent employed; the specific composition employed; the age, body weight, general health, sex and diet of the patient; the time of administration; the route of administration; the rate of excretion of the specific agent employed; the duration of the treatment; drugs used in combination or coincidental with the specific agent employed and like factors well known in the medical arts. It will be understood by a skilled practitioner that the total daily usage of the agents for use in the present invention will be decided by the attending physician within the scope of sound medical judgment.

Agents that decrease the level of functional Aha1, or other related molecules with similar function, can also be used in combination with other therapeutic modalities. Thus, in addition to the therapies described herein, one may also provide to the subject other therapies known to be efficacious for particular protein misfolding diseases.

Controlled-release (or sustained-release) preparations may be formulated to extend the activity of the agent and reduce dosage frequency. Controlled-release preparations can also be used to effect the time of onset of action or other characteristics, such as blood levels of the agent, and consequently affect the occurrence of side effects.

Controlled-release preparations may be designed to initially release an amount of an agent that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized and/or excreted from the body. The controlled-release of an agent may be stimulated by various inducers, e.g., change in pH, change in temperature, enzymes, water, or other physiological conditions or molecules.

Controlled-release systems may include, for example, an infusion pump which may be used to administer the agent in a manner similar to that used for delivering insulin or chemotherapy to specific organs or tumors. Typically, using such a system, the agent is administered in combination with a biodegradable, biocompatible polymeric implant (see below) that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

The agents of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions (see below). Other methods of controlled-release delivery of agents will be known to the skilled artisan and are within the scope of the invention.

Agents that decrease levels of functional Aha1 and/or other related molecules with similar functions can be administered through a variety of routes well known in the arts. Examples include methods involving direct injection (e.g., systemic or stereotactic), implantation of cells engineered to secrete the factor of interest, drug-releasing biomaterials, implantable matrix devices, implantable pumps, injectable gels and hydrogels, liposomes, micelles (e.g., up to 30 □m), nanospheres (e.g., less than 1 □m), microspheres (e.g., 1-100 μm), reservoir devices, etc.

Pulmonary delivery of macromoles and/or drugs, such as the agents described herein, provide for relatively easy, non-invasive administration to the local tissue of the lungs or the circulatory system for systemic circulation (see e.g., Cryan (2004) AAPS J. 7(1) article 4, E20-41, providing a review of pulmonary delivery technology). Advantages of pulmonary delivery include noninvasiveness, large surface area for absorption (˜75 m2), thin (˜0.1 to 0.5 □m) alveolar epitheliuem permitting rapid absorption, absence of first pass metabolism, decreased proteolytic activity, rapid onset of action, and high bioavailablity. Drug formulations for pulmonary delivery, with or without excipients and/or a dispersible liquid, are known to the art. Carrier-based systems for biomolecule delivery, such as polymeric delivery systems, liposomes, and micronized carbohydrates, can be used in conjunction with pulmonary delivery. Penetration enhancers (e.g., surfactants, bile salts, cyclodextrins, enzyme inhibitors (e.g., chymostatin, leupeptin, bacitracin), and carriers (e.g., microspheres and liposomes) can be used to enhance uptake across the alveolar epithelial cells for systemic distribution. Various inhalation delivery devices, such as metered-dose inhalers, nebulizers, and dry-powder inhalers, that can be used to deliver the biomolecules described herein are known to the art (e.g., AErx (Aradigm, Calif.); Respimat (Boehringer, Germany); AeroDose (Aerogen Inc., CA)). As known in the art, device selection can depend upon the state of the biomolecule (e.g., solution or dry powder) to be used, the method and state of storage, the choice of excipients, and the interactions between the formulation and the device. Dry powder inhalation devices are particularly preferred for pulmonary delivery of protein-based agents (e.g., Spinhaler (Fisons Pharmaceuticals, NY); Rotohaler (GSK, NC); Diskhaler (GSK, NC); Spiros (Dura Pharmaceuticals, CA); Nektar (Nektar Pharmaceuticals, CA)). Dry powder formulation of the active biological ingredient to provide good flow, dispersability, and stability is known to those skilled in the art.

Agents affecting a decrease in levels of functional Aha1 can be encapsualted and administered in a variety of carrier delivery systems. Examples of carrier delivery systems include microspheres, hydrogels, polymeric implants, smart ploymeric carriers, and liposomes. Carrier-based systems for biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; and/or improve shelf life of the product.

Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and delivery of the biomolecules described herein. Fabrication, encapsulation, and stabilization of microspheres for a variety of biomolecule payloads are within the skill of the art (see e.g., Varde & Pack (2004) Expert Opin. Biol. 4(1) 35-51). Release rate of microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). Microspheres encapsulating the agents described herein can be administered in a variety of means including parenteral, oral, pulmonary, implantation, and pumping device.

Polymeric hydrogels, composed of hydrophillic polymers such as collagen, fibrin, and alginate, can also be used for the sustained release of agents that decrease levels of functional Aha1 and/or other related molecules with similar function (see generally, Sakiyama et al. (2001) FASEB J. 15, 1300-1302).

Three-dimensional polymeric implants, on the millimeter to centimeter scale, can be loaded with agents that decrease levels of functional Aha1 and/or other related molecules with similar function (see generally, Teng et al (2002) Proc. Natl. Acad. Sci. U.S.A. 99, 3024-3029). A polymeric implant typically provides a larger depot of the bioactive factor. The implants can also be fabricated into structural supports, tailoring the geometry (e.g., shape, size, porosity) to the application. Implantable matrix-based delivery systems are also commercially available in a variety of sizes and delivery profiles (e.g., Innovative Research of America, Sarasota, Fla.).

“Smart” polymeric carriers can be used to administer agents that decrease levels of functional Aha1 and/or other related molecules with similar function (see generally, Stayton et al. (2005) Orthod Craniofacial Res 8, 219-225; Wu et al. (2005) Nature Biotech (2005) 23(9), 1137-1146). Carriers of this type utilize polymers that are hydrophilic and stealth-like at physiological pH, but become hydrophobic and membrane-destabilizing after uptake into the endosomal compartment (i.e., acidic stimuli from endosomal pH gradient) where they enhance the release of the cargo molecule into the cytoplasm. Design of the smart polymeric carrier can incorporate pH-sensing functionalities, hydrophobic membrane-destabilizing groups, versatile conjugation and/or complexation elements to allow the drug incorporation, and an optional cell targeting component. Potential therapeutic macromolecular cargo includes peptides, proteins, antibodies, polynucleotides, plasmid DNA (pDNA), aptamers, antisense oligodeoxynucleotides, silencing RNA, and/or ribozymes that effect a decrease in levels of functional Aha1 and/or related molecules with similar function. As an example, smart polymeric carriers, internalized through receptor mediated endocytosis, can enhance the cytoplasmic delivery of functional Aha1-targeted dsRNA, and/or other agents described herein. Polymeric carriers include, for example, the family of poly(alkylacrylic acid) polymers, specific examples including poly(methylacrylic acid), poly(ethylacrylic acid) (PEAA), poly(propylacrylic acid) (PPAA), and poly(butylacrylic acid) (PBAA), where the alkyl group progressively increased by one methylene group. Smart polymeric carriers with potent pH-responsive, membrane destabilizing activity can be designed to be below the renal excretion size limit. For example, poly(EAA-co-BA-co-PDSA) and poly(PAA-co-BA-co-PDSA) polymers exhibit high hemolytic/membrane destabilizing activity at the low molecular weights of 9 and 12 kDa, respectively. Various linker chemistries are available to provide degradable conjugation sites for proteins, nucleic acids, and/or targeting moieties. For example, pyridyl disulfide acrylate (PDSA) monomer allow efficient conjugation reactions through disulfide linkages that can be reduced in the cytoplasm after endosomal translocation of the therapeutics.

Liposomes can be used to administer agents that decrease levels of functional Aha1 and/or other related molecules with similar function. The drug carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as (phosphatidylcholines), phosphatidylethanolamines (PE), sphingomyelins, phosphatidylserines, phosphatidylglycerols (PG), and phosphatidylinositols (PI). Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used to deliver the biomolecules of the invention. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (e.g., pH-sensitive liposomes) (see e.g., Lasic (1997) Liposomes in Gene Delivery, CRC Press, FL).

Various other delivery systems are known in the art and can be used to administer the agents of the invention. Moreover, these and other delivery systems may be combined and/or modified to optimize the administration of the agents of the present invention.

Pharmaceutical Compositions Comprising dsRNA

In various aspects, the invention provides pharmaceutical compositions comprising a dsRNA, as described herein, and a pharmaceutically acceptable carrier. The pharmaceutical composition comprising the dsRNA is useful for treating a disease or disorder associated with the expression or activity of an Aha gene, such as pathological processes mediated by Aha1 expression. Such pharmaceutical compositions are formulated based on the mode of delivery. One example is compositions that are formulated for systemic administration via parenteral delivery.

The pharmaceutical compositions of the invention are administered in dosages sufficient to inhibit expression of an Aha gene. The present inventors have found that, because of their improved efficiency, compositions comprising the dsRNA of the invention can be administered at surprisingly low dosages. A maximum dosage of 5 mg dsRNA per kilogram body weight of recipient per day is sufficient to inhibit or completely suppress expression of an Aha gene.

In general, a suitable dose of dsRNA will be in the range of 0.01 microgram to 5.0 milligrams per kilogram body weight of the recipient per day, generally in the range of 1 microgram to 1 mg per kilogram body weight per day. The pharmaceutical composition may be administered once daily, or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art and are particularly useful for vaginal delivery of agents, such as could be used with the agents of the present invention. In various aspects, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by Aha expression. Such models are used for in vivo testing of dsRNA, as well as for determining a therapeutically effective dose.

The present invention also includes pharmaceutical compositions and formulations which include the dsRNA compounds of the invention. The pharmaceutical compositions of the present invention may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. For example, topical formulations include those in which the dsRNAs of the invention are in admixture with a topical delivery agent such as lipids, liposomes, fatty acids, fatty acid esters, steroids, chelating agents and surfactants. In various aspects, lipids and liposomes can include neutral (e.g. dioleoylphosphatidyl ethanolamine=DOPE, dimyristoylphosphatidyl choline=DMPC, distearolyphosphatidyl choline) negative (e.g. dimyristoylphosphatidyl glycerol=DMPG) and cationic (e.g. dioleoyltetramethylaminopropyl=DOTAP and dioleoylphosphatidyl ethanolamine=DOTMA). dsRNAs of the invention may be encapsulated within liposomes or may form complexes thereto, in particular to cationic liposomes. Alternatively, dsRNAs may be complexed to lipids, in particular to cationic lipids. In various aspects, fatty acids and esters can include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof.

Those of skill in the art will recognize that compositions and formulations for oral administration can include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations can be those in which dsRNAs of the invention are administered in conjunction with one or more penetration enhancers, surfactants, and chelators. Surfactants can include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Fatty acids can include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Combinations of penetration enhancers, for example, fatty acids/salts in combination with bile acids/salts can be useful. For example, a combination can be the sodium salt of lauric acid, capric acid and UDCA. Further penetration enhancers can include polyoxyethylene-9-lauryl ether, polyoxyethylene-20-cetyl ether.

Those of skill in the art will also recognize that dsRNAs of the invention may be delivered orally, in granular form including sprayed dried particles, or complexed to form micro or nanoparticles. dsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Complexing agents can include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), D EAE-methacrylate, D EAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In various aspects of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Screening

Another aspect of the invention is directed to a system for screening candidate agents for actions on functional Aha1 and/or other related molecules with similar functions, which can be useful for the development of compositions for therapeutic or prophylactic treatment of protein folding diseases. Assays can be performed on living mammalian cells, which more closely approximate the effects of a particular serum level of drug in the body. Cell lines expressing a protein with energetically disfavorable folding characteristics would be useful for evaluating the activity of potential bioactive agents on functional Aha1 and/or other related molecules with similar function, or on extracts prepared from the cultured cell lines. Studies using extracts offer the possibility of a more rigorous determination of direct agent/enzyme interactions.

Thus, the present invention may provide a method to evaluate a agent to decrease the level of functional Aha1 and/or other related molecules with similar functions, and thus to stabilize the folding of proteins with energetically disfavorable folding characteristics in a mammalian host, for example a human host. This assay may comprise contacting the misfolded protein-expressing transgenic cell line or an extract thereof with a preselected amount of the agent in a suitable culture medium or buffer, and measuring the level of functional Aha1 and/or other related molecules with similar functions, as compared to a control cell line or portion of extract in the absence of said agent and/or a control cell line expressing a non-misfolded variant of the protein of interest. For example, screening methods can identify agents that decrease levels of functional Aha1, decrease intracellular Aha1 binding to Hsp90, decrease activation of Hsp90 ATPase, decrease intracellular levels of Hsp90/ATP, and/or increase intracellular levels of Hsp90/ADP.

More specifically, a candidate agent for the treatment of a protein misfolding disease can be screened by providing a cell stably expressing a misfolded protein of interest in a suitable culture medium or buffer, administering the candidate agent to the cell, measuring the levels of functional Aha1 in the cell, and determining whether the candidate agent decreases intracellular functional Aha1 level. Alternatively, a candidate agent for the treatment of a protein misfolding disease can be screened by providing a cell stably expressing a misfolded protein of interest in a suitable culture medium or buffer, administering the candidate agent to the cell, measuring the levels of intracellular Aha1 binding to Hsp90 and/or activation of Hsp90 ATPase, and determining whether the candidate agent decreases such binding and/or activation. Desirable candidates will generally possess the ability to decrease the levels of functional Aha1 in the cell. Provision of a cell stably expressing a misfolded protein is within the skill of the art (see e.g., Examples 1-11).

Any method suitable for detecting levels of functional Aha1 and/or related molecules with similar function, or complexes formed thereto, may be employed for levels resultant from administration of the candidate agent (see e.g., Examples 5-9). Among the traditional methods which may be employed are co-immunoprecipitation, crosslinking, co-purification through gradients or chromatographic columns, and activity assays related to Aha1 function. Utilizing procedures such as these allows for the identification of the proteins and/or complexes of interest.

The agents identified in the screen will generally demonstrate the ability to interact with functional Aha1 and/or related molecules with similar function in such a way as to effect a stabilization of proteins with suboptimal folding kinetics so as to result in increased protein transit. For example, identified agents may decrease levels of functional Aha1, decrease intracellular Aha1 binding to Hsp90, decrease activation of Hsp90 ATPase, decrease intracellular levels of Hsp90/ATP, and/or increase intracellular levels of Hsp90/ADP. These agents can include, but are not limited to, nucleic acids, polypeptides, dsRNAs, antisense molecules, aptamers, ribozymes, triple helices, antibodies, and small inorganic molecules.

Further, the screening methods described above can employ another cell stably expressing a non-misfolded protein variant. By administering the candidate agent, in a substantially similar fashion as to the other cell (expressing a protein with suboptinmal folding kinetics), and measuring the transit level of the non-misfolded protein, one can determine whether the candidate agent substantially decreases the transit level of the non-misfolded protein. Preferably, identified agents do not substantially interfere with folding and/or transit of the non-misfolded protein. Also, a cell stably expressing other proteins can be used similarly to determine whether the agent affects the folding and/or transit of other related or unrelated proteins. For example, an identified agent that decreases levels of functional Aha1 preferably does not significantly impair transit of other proteins with more energetically stable folds.

The invention also encompasses methods for identifying agents that specifically bind to functional Aha1 and/or other related molecules with similar function. One such method involves the steps of providing immobilized purified functional Aha1 protein and at least one test agent; contacting the immobilized protein with the test agent; washing away agents not bound to the immobilized protein; and detecting whether or not the test agent is bound to the immobilized protein. Those agents remaining bound to the immobilized protein are those that specifically interact with the functional Aha1 protein.

The present invention also comprises the use of functional Aha1 (and/or other molecules with similar function) in drug discovery efforts to elucidate relationships that exist between functional Aha1 (and/or other molecules with similar function) and a disease state, phenotype, or condition, such as protein misfolding diseases. These methods include detecting or decreasing levels of Aha1 polynucleotides comprising contacting a sample, tissue, cell, or organism with the agents of the present invention, measuring the nucleic acid or protein level of functional Aha1, and/or a related phenotypic or chemical endpoint at some time after treatment, and optionally comparing the measured value to a non-treated sample or sample treated with a further agent of the invention. These methods can also be performed in parallel or in combination with other experiments to determine the function of unknown genes for the process of target validation or to determine the validity of a particular gene product as a target for treatment or prevention of a particular disease, condition, or phenotype.

Therapeutic Treatment

One aspect of the invention provides methods of treatment for protein folding diseases. Without being bound by a particular theory, it is possible that decreasing functional Aha1 levels, and hence decreasing Hsp90 ATPase activity, may allow additional time for the kinetically challenged ΔF508 mutant to utilize the rescue chaperone to create a more export competent fold. Protein folding can, therefore, be treated in a subject in need thereof by administering an agent that decreases the level of functional Aha1 and/or other related molecules having similar function.

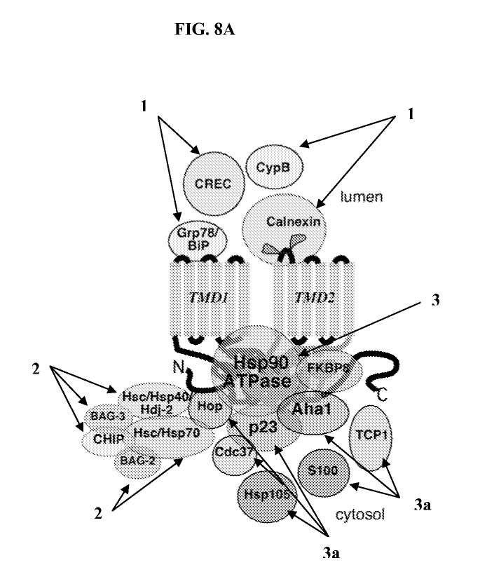

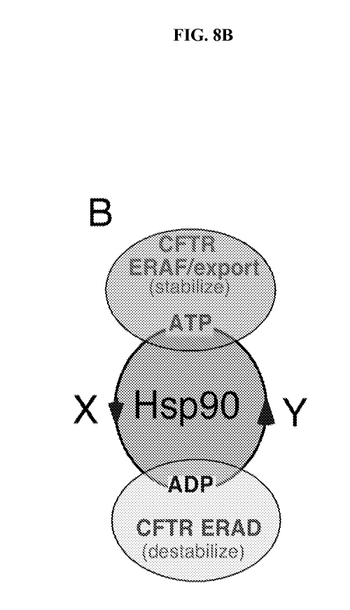

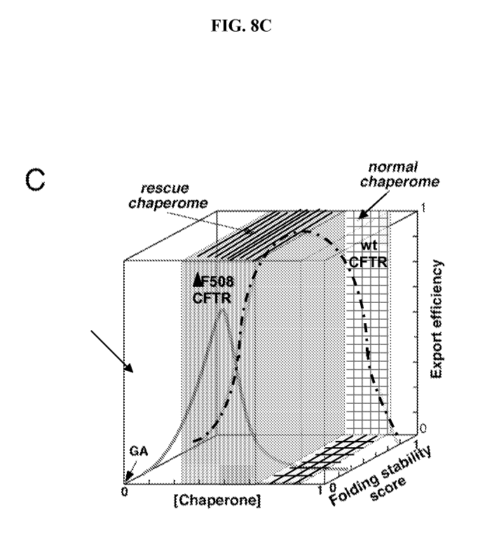

Further, and again without being bound by a particular theory, it is possible that the Hsp90-ADP-state favors a link of cargo and ERAD pathways, whereas the Hsp90-ATP-state affords coupling to COPII based on the response to functional Aha1 (see e.g., FIG. 8B, X) or p23 (see e.g., FIG. 8B, Y) given their known biochemical properties. Thus, another approach for prophylactic or therapeutic treatment of a protein misfolding disease can involve administering to a subject in need thereof an agent that decreases binding of a functional Aha1 to Hsp90 ATPase and/or decreases resulting activation levels resulting from binding of functional Aha1 to Hsp90 ATPase.

Preferably, administration of the agent does not substantially interfere with folding and/or transit of other intracellular proteins. For example, administration of an agent that decrease levels of functional Aha1, decreases binding of functional Aha1 to Hsp90 ATPase, and/or decreases resulting activation levels resulting from binding of functional Aha1 to Hsp90 ATPase to treat a protein misfolding disease preferably does not significantly impair transit of other proteins, for example, other proteins with more energetically stable folds.

Disease states or conditions indicative of a need for therapy in the context of the present invention, and/or amenable to treatment methodologies described herein, include protein misfolding diseases such as CF, marfan syndrome, Fabry disease, Gaucher's disease, retinitis pigmentosa 3, Alzheimer's disease, Type II diabetes, Parkinson's disease, spongiform encephalopathies such as Creutzfeldt-Jakob disease, primary systemic amyloidosis, secondary systemic amyloidosis, senile systemic amyloidodis, familial amyloid polyneuropathy 1, hereditary cerebral amyloid angiopathy, hemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, and hereditary renal amyloidosis. For example, protein misfolding diseases treatable according to methods described herein include those diseases where misfolded proteins result in decreased protein transit from the ER and increased protein degradation, such as CF, marfan syndrome, Fabry disease, Gaucher's disease, and retinitis pigmentosa 3. As another example, protein misfolding diseases treatable according to methods described herein include those diseases where misfolded proteins result in deposition of insoluble aggregates, such as Alzheimer's disease, Type II diabetes, Parkinson's disease, and spongiform encephalopathies (e.g., Creutzfeldt-Jakob disease). The protein misfolding diseases listed above can be caused, at least in part, by misfolded of CFTR, fibrillin, alpha galactosidase, beta glucocerebrosidase, rhodopsin, amyloid beta and tau (islet amyloid polypeptide), amylin, alpha synuclein, prion, immunoglobulin light chain, serum amyloid A, transthyretin, cystatin C, β2-microglobulin, apolipoprotein A-1, gelsolin, calcitonin, atrial natriuretic factor, lysozyme, insulin, and fibrinogen.

A determination of the need for treatment will typically be assessed by a history and physical exam consistent with the disease. For example, the diagnosis of CF can involve a combination of clinical criteria and analysis of sweat Cl-values. In addition, DNA analysis for ΔF508 can be performed. Such CF diagnosis is within the skill of the art (see e.g., Cutting (2005) Annu Rev Genomics Hum Genet. 6, 237-260, reviewing CF). Subjects with an identified need of therapy include those with a diagnosed protein misfolding disease or indication of a protein misfolding disease amenable to therapeutic treatment described herein and subjects who have been treated, are being treated, or will be treated for a protein misfolding disease. The subject is preferably an animal, including, but not limited to, mammals, reptiles, and avians, more preferably horses, cows, dogs, cats, sheep, pigs, and chickens, and most preferably human.

Another aspect of the invention is directed toward rescuing a cell from the effects of protein misfolding. Such approach is directed to cellular function and can be performed in vitro, in vivo, or ex vivo. As an example, rescue of a cell from the effect of protein misfolding can occur in a cell from a cultured cell line. As another example, rescue of a cell from the effect of protein misfolding can occur in a cell removed from a subject and then subsequently reintroduced to the subject. As a further example, rescue of a cell from the effect of protein misfolding can occur in a cell of the subject in situ. Administration of an agent that decreases levels of functional Aha1 and/or related molecules with similar function to a cell wherein protein misfolding occurs can facilitate stabilization of energetically unstable folds, resulting in rescue of impaired intracellular and/or extracellular transit of the protein. For example, administration to a cell expressing misfolded ΔF508 CFTR of an agent that reduces levels of the Hsp90 co-chaperone and functional Aha1 can enhance ΔF508 ER stability, rescue ΔF508 trafficking to the cell surface, increase cell surface ΔF508 availability, and/or at least partially restore channel function (see e.g., Example 10). Preferably, administration of an agent to decrease levels of functional Aha1 to rescue a cell from the effects of protein misfolding preferably does not substantially interfere with folding and/or transit of other intracellular proteins.

Therapeutic Treatment Using dsRNA

The invention relates in particular to the use of a dsRNA or a pharmaceutical composition prepared therefrom for the treatment of Cystic Fibrosis. Owing to the inhibitory effect on Aha1 expression, a dsRNA according to the invention or a pharmaceutical composition prepared therefrom can enhance the quality of life of Cystic Fibrosis patients.

Furthermore, the invention relates to the use of a dsRNA or a pharmaceutical composition of the invention aimed at the treatment of cancer, e.g., for inhibiting tumor growth and tumor metastasis. For example, the dsRNA or a pharmaceutical composition prepared therefrom may be used for the treatment of solid tumors, like breast cancer, lung cancer, head and neck cancer, brain cancer, abdominal cancer, colon cancer, colorectal cancer, esophagus cancer, gastrointestinal cancer, glioma, liver cancer, tongue cancer, neuroblastoma, osteosarcoma, ovarian cancer, pancreatic cancer, prostate cancer, retinoblastoma, Wilm's tumor, multiple myeloma and for the treatment of skin cancer, like melanoma, for the treatment of lymphomas and blood cancer. The invention further relates to the use of an dsRNA according to the invention or a pharmaceutical composition prepared therefrom for inhibiting Aha1 expression and/or for inhibiting accumulation of ascites fluid and pleural effusion in different types of cancer, e.g., breast cancer, lung cancer, head cancer, neck cancer, brain cancer, abdominal cancer, colon cancer, colorectal cancer, esophagus cancer, gastrointestinal cancer, glioma, liver cancer, tongue cancer, neuroblastoma, osteosarcoma, ovarian cancer, pancreatic cancer, prostate cancer, retinoblastoma, Wilm's tumor, multiple myeloma, skin cancer, melanoma, lymphomas and blood cancer. Owing to the inhibitory effect on Aha1 expression, a dsRNA according to the invention or a pharmaceutical composition prepared therefrom can enhance the quality of life of cancer patients.

The invention furthermore relates to the use of an dsRNA or a pharmaceutical composition thereof, e.g., for treating Cystic Fibrosis or cancer or for preventing tumor metastasis, in combination with other pharmaceuticals and/or other therapeutic methods, e.g., with known pharmaceuticals and/or known therapeutic methods, such as, for example, those which are currently employed for treating Cystic Fibrosis or cancer and/or for preventing tumor metastasis. Where the pharmaceutical composition aims for the treatment of Cystic fibrosis, the composition can be, for example, given to a combination with daily chest physiotherapy, orally applied pancreatic enzymes, daily oral or inhaled antibiotics to counter lung infection, inhaled anti-asthma therapy, corticosteroid tablets, dietary vitamin supplements, especially A and D, inhalation of Pulmozyme medicines to relieve constipation or to improve the activity of the enzyme supplements, insulin for CF-related diabetes, medication for CF-associated liver disease, and oxygen to help with breathing.

Where the pharmaceutical composition aims for the treatment of cancer and/or for preventing tumor metastasis, the composition can be, for example, given to a combination with radiation therapy and chemotherapeutic agents, such as cisplatin, cyclophosphamide, 5-fluorouracil, adriamycin, daunorubicin or tamoxifen.

The invention can also be practiced by including with a specific RNAi agent another anti-cancer chemotherapeutic agent, such as any conventional chemotherapeutic agent. The combination of a specific binding agent with such other agents can potentiate the chemotherapeutic protocol. Numerous chemotherapeutic protocols will present themselves in the mind of the skilled practitioner as being capable of incorporation into the method of the invention. Any chemotherapeutic agent can be used, including alkylating agents, antimetabolites, hormones and antagonists, radioisotopes, as well as natural products. For example, the compound of the invention can be administered with antibiotics such as doxorubicin and other anthracycline analogs, nitrogen mustards such as cyclophosphamide, pyrimidine analogs such as 5-fluorouracil, cisplatin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of a tetracycline compound with another treatment modality, e.g., surgery, radiation, etc., also referred to herein as “adjunct antineoplastic modalities.” Thus, the method of the invention can be employed with such conventional regimens with the benefit of reducing side effects and enhancing efficacy.

### EXAMPLES

Aspects of the present teachings may be further understood in light of the following examples, which should not be construed as limiting the scope of the present teachings in any way.

### Example 1

### CFTR Interactome

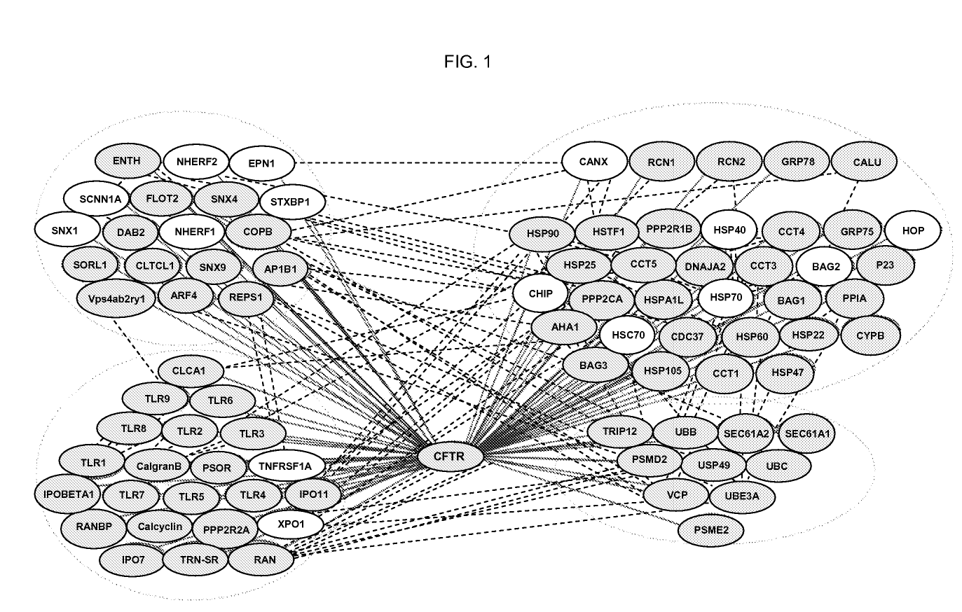

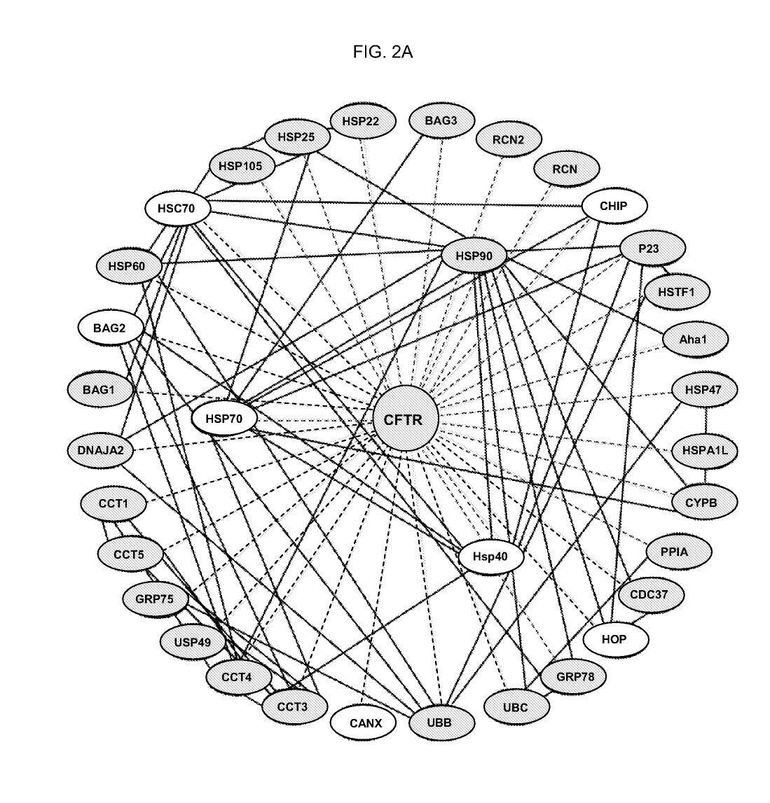

To define global protein interactions involved in CFTR trafficking and function in the exocytic and endocytic pathways, CFTR-containing protein complexes were immunoisolated from cell lines expressing wild-type CFTR (see e.g., FIG. 1), protease digested, and the composition of the peptide mixture determined using multidimensional protein identification technology (MudPIT) (Lin et al., Biochim Biophys Acta 1646, 1 (2003)).

CFTR was immunoprecipitated from stable BHK cell lines over-expressing either wild-type or ΔF508 CFTR, or the Calu-3, HT29 and T84 cell lines expressing wild-type CFTR. Baby Hamster Kidney (BHK) cells stably expressing wt or ΔF508 CFTR were maintained in DMEM supplemented with F12, 5% fetal bovine serum (FBS), 100 units/ml each of penicillin and streptomycin (Pen/Strep), and 500 μM methotrexate (Xanodyne Pharmacal, Inc., Florence, Ky.). Parental BHK cells not expressing CFTR were cultured in the same medium except without methotrexate. Human lung cell line Calu-3, and human intestinal cell lines HT29 and T84, all expressing endogenous wt CFTR were purchased from ATCC and maintained according to manufacturer's instructions.

CFTR and co-immunoprecipitating proteins in whole cell detergent lysates were bound to Sepharose beads coupled with the anti-CFTR monoclonal antibody M3A7. To capture transient or weak interactions that occur as CFTR transits through different subcellular compartments, immunoprecipitations were carried out in the absence or presence of the cleavable chemical cross-linker dithiobissuccinimidylpropionate (DSP) that was added to intact cells prior to cell lysis. To control for non-specific binding of proteins to beads, several conditions were used to indicate background recoveries including incubation of cell lysates in the presence beads alone, or beads coupled to a monoclonal antibody directed against the VSV-G, a protein only found in cells infected with vesicular stomatitis virus (Calu-3, T84 and H89 datasets). In the case of BHK cells, immunoprecipitates from wild-type and ΔF508 expressing stable cell lines was compared directly to the parent cell line not expressing CFTR. In the datasets provided in the Excel files, recovered proteins from both non-cross-linked and cross-linked methods are pooled.

Following immunoprecipitation, protein complexes were digested by denaturing the proteins in freshly prepared 8 M guanidine HCl followed by dilution to 2 M. Endoproteinase LysC was used to digest the proteins for 8 hours, followed by dilution to 1 M guanidine HCl and trypsin digestion using Porozyme™ trypsin beads. All digestions are performed at 37° C.

Protease digested immune complexes were subjected to LC/LC/MS/MS analysis using MudPIT. This approach has been described in detail by several authors (Link et al., 1999 Nat Biotechnol 17, 676-682; MacCoss et al., 2002, Proc Natl Acad Sci USA 99, 7900-7905; MacCoss et al., 2002, Anal Chem 74, 5593-5599; McDonald et al., 2002 Int J Mass Spect 219, 245-251; Washburn et al., 2001 Nat Biotechnol 19, 242-247). Briefly, the denatured, reduced and alkylated proteins were split into three fractions and digested over night at 37° C. with three different proteases (trypsin, subtylisin and elastase). The resulting peptide mixture was acidified with formic acid (5%). Subsequently, a three phase microcapillary column was constructed by slurry packing ˜7 cm of 5-μm Aqua C18 material (Aqua, Phenomimex) into a 100 μm fused silica capillary, which had been previously pulled to a tip diameter of ˜5 μm using a Sutter Instruments laser puller (Sutter Manufacturing, Novato, Calif.). Next, 3 cm of 5-μm Partisphere strong cation exchange resin (Partisphere, Whatman) followed by another 3 cm of 5-μm Aqua C18 chromatography material was packed into the column. The column was then equilibrated with 5% acetonitrile/0.1% formic acid for ˜30 min before the peptide mixture was loaded onto the back-end of a triphasic chromatography column using a high pressure cell.

After loading the peptide digests, the column was placed inline with an Agilent 1100 quaternary HPLC and analyzed using a modified 6-step separation. The buffer solutions were 5% acetonitrile/0.1% formic acid (buffer A), 80% acetonitrile/0.1% formic acid (buffer B), and 500 mM ammonium acetate/5% acetonitrile/0.1% formic acid (buffer C). Step I consisted of a 100 min gradient from 0-100% buffer B. Steps 2-5 had the following profile: 3 min of 100% buffer A, 2 min of X % buffer C, a 10 min gradient from 0-15% buffer B, and a 97 min gradient from 15-45% buffer B. The 2 min buffer C percentages (X) were 10, 20, 30, 40% respectively for the 6-step analysis. In the final step, the gradient contained: 3 min of 100% buffer A, 20 min of 100% buffer C, a 10 min gradient from 0-15% buffer B, and a 107 min gradient from 15-70% buffer B.

As peptides eluted from the microcapillary column, they were electrosprayed directly into an LCQ-Deca mass spectrometer with the application of a distal 2.4 kV spray voltage. A cycle of one full-scan mass spectrum (400-1400 m/z) followed by 3 data-dependent MS/MS spectra at a 35% normalized collision energy was repeated continuously throughout each step of the multidimensional separation. Application of mass spectrometer scan functions and HPLC solvent gradients are controlled by the Xcalibur data system.

Tandem mass spectra were analyzed sequentially using the following protocol. First, a software algorithm (2 to 3) was used to determine the appropriate charge state (either +2 or +3) from multiple charged peptide mass spectra, and delete spectra of poor quality (Sadygov et al., 2002, J Proteome Res 1, 211-215). The MS/MS spectra after 2 to 3 was searched using a parallel virtual machine (PVM) version of SEQUEST™ (Yates et al., 1995, Anal Chem 67, 3202-3210; Yates et al., 1995, Anal Chem 67, 1426-1436) running on a Beowolf computer cluster (˜75 cpu's) against a protein database constructed from the combined human, mouse and rat databases (HMR) from Refseq. Database search results were filtered, sorted, and displayed using the DTASelect program (Tabb et al., 2002, J Proteome Res 1, 21-26). Default DTASelect criteria were employed (i.e., +1 1.8, +2 2.5, +3 3.5, ΔCN 0.08, and at least two peptides per locus).

The protein components identified from replicate immuonprecipitations were merged and the resulting dataset was then annotated with the GO_Annotation using the EASE analysis program supplied by the NIH (Hosack et al., 2003, Genome Biol 4, R70). From the control-subtracted proteome, proteins which were annotated by GO_Molecular_Function as belonging to the nucleic acid binding, or structural categories which are common contaminants from whole cell proteomic experiments, were eliminated. The resulting list of proteins primarily included cytoplasmic chaperones, endoplasmic reticulum (ER) lumenal proteins, late secretory pathway components, cell surface interactors, and proteosome/ubiquitination components.

FIG. 1 is a cartoon depicting components comprising the CFTR interactome (light ovals, previously established interactions; dark ovals, new interactions recovered in the current study) as nodes in the network and are divided into subnetworks that potentially facilitate protein folding in the ER (I), ERAD (II), membrane trafficking (III), and post-ER regulators and effectors (IV). Dark lines are edges in the network that show direct or indirect protein interactions between CFTR and the indicated component identified by MudPIT. Light gray lines illustrate edges that define interactions based on the Tmm co-expression database, accessed using the Cytoscape platform. Table 7 shows the results of an array conducted on proteins recovered using multidimensional protein identification technology (MudPIT) in the indicated cell types expressing wild-type CFTR, arranged in the order of fractional sequence coverage by mass spectrometry.

Results showed that the identified protein generate a network of protein interactions defining the CFTR proteome or interactome (see e.g., FIG. 1). Proteins comprising the CFTR interactome can be divided into subnetworks that collectively define functional groups that include components required for folding and export from the ER (see e.g., FIG. 1-I), that mediate ERAD (see e.g., FIG. 1-II), that direct transport between the exocytic and endocytic compartments (see e.g., FIG. 1-III), and components that are potential binding partners involved in CFTR function and regulation at the cell surface (see e.g., FIG. 1B-IV).

A number of protein interactions found for mature wild-type CFTR found at the cell surface validate the database (see e.g., Table 8). For example, CFTR is a gated chloride channel whose activity is regulated by cAMP-dependent protein kinases and protein phosphatases (Guggino and Banks-Schlegel, 2004). Protein phosphatase 2A (PP2A) or PP2C have a role in CFTR dephosphorylation and down-regulation of CFTR activity in a variety of cell types. Although kinases were not detected as a stable interacting partners in any cell line examined, presumably because of their very transient interaction, wild-type CFTR in nearly all cell lines showed strong interaction with PP2A-both the regulatory and catalytic subunits (Thelin et al., 2005; Vastiau et al., 2005). In addition to PP2A, sodium-hydrogen exchanger (NHE) isoform 3 regulators 1 and 3 (NHERF-1/3) (Mohler et al., 1999; Yun et al., 1997) were recovered in the CFTR proteome. NHERFs are localized to the apical surface of lung cells and are well-documented to interact with CFTR through the C-terminal PDZ domains (Guggino and Banks-Schlegel, 2004, Am J Respir Crit. Care Med 170, 815-820). A previous unknown interactor includes calgranulin B (S100-A8), a member of the divergent S100 family of EF-hand-containing cytosolic Ca″ binding proteins (Donato, 2003, Microsc Res Tech 60, 540-551; Heizmann, 2002, Methods Mol Biol 172, 69-80). Calgranulin B has been implicated in CF inflammatory pathways (Fanjul et al., 1995, Am J Physiol 268, C1241-1251; Renaud et al., 1994, Biochem Biophys Res Commun 201, 1518-1525; Xu et al., 2003, J Biol Chem 278, 7674-7682), suggesting a possible modulatory role related to CFTR lung pathophysiology.

Results also showed that, generally, the identification of multiple endocytic trafficking components illustrate the importance of CFTR internalization and recycling in normal function. A second group of components highlights direct or indirect interactions of wild-type CFTR with the membrane trafficking machinery (see e.g., Table 7). These include sortilinrelated receptor L (SORL1), disabled homolog 2 (Dab2), RaIBP1 associated Eps domain containing protein (Reps 1), ARF4, clathrin light chain, vacuolar sorting protein 4 (Vps4p), enthoprotin, and sorting nexins (SNX) 4 and 9. SORL1 has a single transmembrane domain, is localized to recycling endosomes and involved in internalization of multiple ligands (Jacobsen et al., 2001, J Biol Chem 276, 22788-22796). Dab2 functions as a cargo-selective endocytic clathrin adaptor (Bonifacino and Traub, 2003, Annu Rev Biochem 72, 395-447; Mishra et al., 2002, Embo J 21, 4915-4926), whereas Repsl is able to bind to proteins containing the NPF internalization motif found in CFTR (Yamaguchi et al., 1997, J Biol Chem 272, 31230-31234) and couple to the Rab 1′-FIP2 family of endocytic GTPase regulators (Bilan et al., 2004, J Cell Sci 117, 1923-1935; Cullis et al., 2002, J Biol Chem 277, 49158-49166; Gentzsch et al., 2004, Mol Biol Cell 15, 2684-2696; Swiatecka-Urban et al., 2005, J Biol Chem 280, 36762-36772) by a cargo selection machinery containing AP2, Dab2. ARF4 is a small GTPase implicated in endocytic/recycling compartments (Donaldson and Honda, 2005, Biochem Soc Trans 33, 639-642; Langhorst et al., 2005, Cell Mol Life Sci 62, 2228-2240; Morrow and Parton, 2005, Traffic 6, 725-740), whereas VPS4 likely functions in the transport of proteins from late endosomal compartments to the lysosome (Bowers et al., 2004, Traffic 5, 194-210; Hislop et al., 2004, J Biol Chem 279, 22522-22531; Scheuring et al., 2001, J Mol Biol 312, 469-480; Scott et al., 2005, Embo J 24, 3658-3669). Enthoprotin interacts with clathrin adaptor API, with the Golgi-localized y-ear containing, ARF-binding protein 2 (McPherson and Ritter, 2005, Mol Neurobiol 32, 73-87; Wasiak et al., 2003, FEBS Lett 555, 437-442; Wasiak et al., 2002, J Cell Biol 158, 855-862), and through its carboxyl terminal domain, to the terminal domain of clathrin heavy chain to stimulate the formation of clathrin-coated vesicle (Kalthoff et al., 2002, Mol Biol Cell 13, 4060-4073; Wasiak et al., 2003, supra; Wasiak et al., 2002, supra), consistent with the recovery of the clathrin light chain (Ybe et al., 2003, Traffic 4, 850-856) and the established role for clathrin in CFTR recycling (Cheng et al., 2004, J Biol Chem 279, 1892-1898; Hu et al., 2001, Biochem J 354, 561-572; Lukacs et al., 1997, Biochem J 328 (Pt 2), 353-361; Peter et al., 2002, J Biol Chem 277, 49952-49957; Picciano et al., 2003, Am J Physiol Cell Physiol 285, C1009-1018; Weixel and Bradbury, 2000, J Biol Chem 275, 3655-3660; Weixel and Bradbury, 2001, Pflugers Arch 443 Suppl 1, S70-74; Weixel and Bradbury, 2001, J Biol Chem 276, 46251-46259). Additional adaptors identified in the interactome include Snx4 and Snx9 (Carlton et al., 2005, Traffic 6, 75-82; Lundmark and Carlsson, 2003, J Biol Chem 278, 46772-46781; Wasiak et al., 2003, J Cell Biol 158, 855-862). Snx9 binds the β-appendage domain of AP2 and assists AP2 in its function at the plasma membrane in clathrin and dynamin mediated internalization (Lin et al., 2002, supra; Lundmark and Carlsson, 2003, supra; Lundmark and Carlsson, 2004, J Biol Chem 279, 42694-42702; Lundmark and Carlsson, 2005, Methods Enzymol 404, 545-556; Soulet et al., 2005, Mol Biol Cell 16, 2058-2067; Teasdale et al., 2001, Biochem J 358, 7-16). Snx4 has been reported to interact with amphiphysin to facilitate endocytic trafficking of transferrin and other recycling components (Hettema et al., 2003, Embo J 22, 548-557; Leprince et al., 2003, J Cell Sci 116, 1937-1948).

While the above results focus on effector and trafficking components, both wild-type and ΔF508-CFTR are degraded by ERAD pathways that involve both ubiquitin and proteasome components (Amaral, 2004, J Mol Neurosci 23, 41-48) (see e.g., Table 8). The proteome from both wild-type and ΔF508 CFTR expressing cells contain components involved in ERAD. These include the translocation/dislocation Sec61 channel and VCP/p97/Cdc48, a chaperone directing delivery to the proteasome. The role of the proteasome is indicated by the enrichment in 26S proteasome subunits and components of the ubiquitination pathway in the interactome (see e.g., Table 8). Interestingly, the ubiquitinating-conjugating protein E3A recovered in the proteome shows interaction with Ubc6, a class of E2 ubiquitin-conjugating enzymes frequently invoked for ERAD, including CFTR (Lenk et al., 2002, J Cell Sci 115, 3007-3014).

Whether these ligases are only involved in ERAD at the level of the ER or also participate in down-regulation of CFTR at the cell surface through endocytic/lysosomal targeting pathways remains to be determined (Gentzsch et al., 2004, Mol Biol Cell; Sharma et al., 2004, J Cell Biol 164, 923-933; Swiatecka-Urban et al., 2005, supra). In addition to those examples discussed above, additional components are predicted to have direct or indirect interactions with CFTR (see e.g., Tables 1 and 2).

The above is a systems biology approach aided by the sensitivity of the MudPIT proteomics technology taken to identify transient interactions that contribute to CFTR folding and trafficking pathways, the CFTR interactome. While some proteins that have been shown to interact with CFTR in post-ER compartments were not identified, this could reflect limitations of the mass spectometry technique, the immunoprecipitation conditions optimized for consistency within the study, and the fact that the interactome is likely composed of very dynamic, and therefore, transient interactions that are difficult to capture and highly depended on cell type and growth conditions. The interactome encompassing all known interactions (see e.g., FIG. 1) can provide a new baseline to begin to assess the many different protein complexes necessary for CFTR to achieve and maintain functionally at the apical cell surface.

TABLE 5
Post-ER CFTR interacting proteins of known function*

| | | | | |
|---|---|---|---|---|
| Reference Sequence | | | | |
| accession code | Protein name | % cov. | unique | total |

Cell surface transporters and regulators

| | | | | |
|---|---|---|---|---|
| NM_004252 | NHERF-1** | 20% | 5 | 6 |
| NM_004785 | NHERF-2*** | 14% | 4 | 5 |
| NM_001285 | CLCA1 | 2% | 2 | 2 |

Components of post-ER trafficking machinery

| | | | | |
|---|---|---|---|---|
| NM_003105 | SORL1 | 1% | 1 | 12 |
| NM_016760 | clathrin, light chain | 13% | 3 | 11 |
| NM_003794 | sorting nexin 4 | 3% | 3 | 5 |
| NM_014666 | enthoprotin | 9% | 4 | 5 |
| NM_007479 | ARF4 | 16% | 2 | 4 |
| NM_023118 | Dab2 | 5% | 3 | 3 |
| NM_016451 | βCOP | 5% | 2 | 2 |
| NM_013245 | VPS 4A | 5% | 2 | 2 |
| NM_009503 | VCP | 7% | 2 | 2 |
| NM_009048 | Reps1 | 6% | 2 | 2 |
| NM_016224 | sorting nexin 9 | 5% | 2 | 2 |
| NM_008028 | flotillin 2 | 5% | 2 | 2 |

*Shown are the percentage sequence coverage (% coverage), number of unique peptide spectra (unique), and number of total peptide spectra (total) as identified by mass spectrometry.
**Shown are the peptide data for BHK cells. Those for HT29 are 11%, 2, 2.
***Shown are peptide data for Calu-3 cells. Those for HT29 are 20%, 7, 10.

TABLE 6
Degradation proteome associated with CFTR

| Reference | | | | | | | |
|---|---|---|---|---|---|---|---|
| Sequence | | | | | | | |
| accesssion | | ΔF508 | | | WT^{#} | | |
| code | Protein name | A* | B* | C* | A* | B* | C* |
| NM_017314 | ubiquitin C | 6% | 5 | 117 | 5% | 3 | 36 |
| NM_011664 | ubiquitin B | 16% | 5 | 52 | 12% | 3 | 16 |
| NM_007126 | VCP/p97/Cdc48 | 25% | 10 | 14 | 7% | 2 | 2 |
| NM_002808 | proteasome 26S, non-ATPase, 2 | 13% | 7 | 8 | 4% | 2 | 2 |
| NM_008944 | proteasome, alpha type 2 | 17% | 2 | 5 | | | |
| NM_002795 | proteasome, beta type, 3 | 24% | 3 | 3 | | | |
| NM_011185 | proteasome, beta type 1 | 23% | 3 | 3 | | | |
| NM_011967 | proteasome, alpha type 5 | 23% | 3 | 3 | | | |
| NM_006503 | proteasome 26S, ATPase, 4 | 12% | 2 | 3 | | | |
| NM_008948 | proteasome 26S, ATPase 3 | 8% | 2 | 3 | | | |
| NM_002796 | proteasome, beta type, 4 | 16% | 2 | 2 | | | |
| NM_002815 | proteasome 26S, non-ATPase, 11 | 5% | 2 | 2 | | | |
| NM_013336 | Sec61 alpha subunit isoform 1 | | | | 11% | 4 | 7 |
| NM_004652 | ubiquitin specific protease 9 | | | | 1% | 2 | 3 |
| NM_000462 | ubiquitin protein ligase E3A | | | | 6% | 2 | 2 |
| NM_004238 | THR interactor 12 | | | | 2% | 2 | 2 |
| NM_018144 | Sec61, alpha subunit 2 | | | | 11% | 2 | 2 |

^{#}Proteins recovered from BHK(wild-type CFTR), Calu-3, HT29, T84 cell lines
*Shown are the percentage sequence coverage (A), number of unique peptide spectra (B), and number of total peptide spectra (C) as identified by mass spectrometry.

TABLE 7
CFTR proteome for cell lines expressing wild-type CFTR

| | | | Sequence Coverage (%) | | | |
|---|---|---|---|---|---|---|
| | | | BHK | | | |
| Protein# | RefSeq AC | gene name | wt | Calu-3 | HT29 | T84 |
| 1 | NM_000492 | cystic fibrosis | 0.599 | 0.526 | 0.455 | 0.407 |
| | | transmembrane | | | | |
| | | conductance regulator | | | | |
| 2 | NM_008379 | karyopherin (importin) beta 1 | 0.579 | 0.287 | 0.068 | 0.075 |
| 3 | NM_009037 | reticulocalbin | 0.495 | | | |
| 4 | NM_006597 | Hsc70 | 0.466 | | 0.375 | 0.354 |
| 5 | NM_001539 | Hsp40-A1 (Hdj2) | 0.403 | 0.081 | 0.113 | |
| 6 | NM_006391 | importin 7 | 0.334 | 0.078 | 0.046 | |
| 7 | NM_022310 | GRP78 | 0.328 | 0.188 | 0.137 | |
| 8 | NM_005507 | cofilin 1 (non-muscle) | 0.307 | | | |
| 9 | NM_005880 | Hsp40-A2 (Hdj3) | 0.282 | | 0.133 | |
| 10 | NM_002715 | protein phosphatase 2 | 0.275 | 0.11 | | |
| | | (formerly 2A), catalytic | | | | |
| | | subunit, alpha isoform | | | | |
| 11 | NM_001316 | CSE1 chromosome | 0.255 | 0.143 | 0.048 | |
| | | segregation 1-like (yeast) | | | | |
| 12 | NM_002717 | protein phosphatase 2 | 0.248 | | | |
| | | (formerly 2A), regulatory | | | | |
| | | subunit B (PR 52), alpha | | | | |
| | | isoform | | | | |
| 14 | NM_011992 | reticulocalbin 2 | 0.221 | | | |
| 15 | NM_002901 | reticulocalbin 1, EF-hand | 0.215 | 0.145 | | |
| | | calcium binding domain | | | | |
| 16 | NM_008302 | Hsp90 beta | 0.214 | 0.171 | 0.076 | |
| 17 | NM_012030 | NHERF-1 | 0.197 | | 0.112 | |
| 18 | NM_006098 | guanine nucleotide binding | 0.189 | | | |
| | | protein (G protein), beta | | | | |
| | | polypeptide 2-like 1 | | | | |
| 19 | NM_002902 | reticulocalbin 2, EF-hand | 0.183 | 0.186 | 0.123 | |
| | | calcium binding domain | | | | |
| 20 | NM_011313 | S100 calcium binding | 0.18 | | | |
| | | protein A6 (calcyclin) | | | | |
| 22 | NM_018243 | hypothetical protein | 0.175 | | | |
| | | FLJ10849 | | | | |
| 23 | NM_009906 | ceroid-lipofuscinosis, | 0.16 | | | |
| | | neuronal 2 | | | | |
| 24 | NM_002882 | RAN binding protein 1 | 0.159 | | | |
| 25 | NM_007479 | ADP-ribosylation factor 4 | 0.156 | | | |
| 26 | NM_003400 | exportin 1 (CRM1 | 0.154 | 0.093 | 0.053 | 0.142 |
| | | homolog, yeast) | | | | |
| 27 | NM_002716 | protein phosphatase 2 | 0.151 | | | |
| | | (formerly 2A), regulatory | | | | |
| | | subunit A (PR 65), beta | | | | |
| | | isoform | | | | |
| 28 | NM_001681 | SERCA2 | 0.15 | | | 0.092 |
| 29 | NM_021594 | ERM-binding | 0.149 | | | |
| | | phosphoprotein | | | | |
| 30 | NM_005998 | chaperonin containing | 0.14 | | | |
| | | TCP1, subunit 3 (gamma) | | | | |
| 31 | NM_007637 | chaperonin subunit 5 | 0.131 | | | |
| | | (epsilon) | | | | |
| 32 | NM_011664 | ubiquitin B | 0.121 | 0.072 | | |
| 33 | NM_021671 | db83 | 0.117 | | | |
| 34 | NM_013336 | protein transport protein | 0.111 | | 0.113 | 0.099 |
| | | SEC61 alpha subunit | | | | |
| | | isoform 1 | | | | |
| 35 | NM_028152 | MMS19 (MET18 S. cerevisiae)- | 0.11 | | | |
| | | like | | | | |
| 36 | NM_004282 | BAG-2 | 0.104 | | | |
| 37 | NM_001746 | calnexin | 0.096 | | | 0.095 |
| 38 | NM_012470 | transportin-SR | 0.094 | | | |
| 39 | NM_025291 | steroid receptor RNA | 0.091 | | | |
| | | activator 1 | | | | |
| 40 | NM_013686 | TCP1 | 0.09 | | | |
| 41 | NM_005345 | Hsp70-1A | 0.083 | | 0.315 | 0.083 |
| 42 | NM_020645 | chromosome 11 open | 0.083 | | | |
| | | reading frame 14 | | | | |
| 43 | NM_018307 | ras homolog gene family, | 0.081 | | | |
| | | member T1 | | | | |
| 44 | NM_021979 | Hsp70-2 | 0.078 | 0.128 | 0.102 | 0.078 |
| 45 | NM_001219 | calumenin | 0.07 | 0.111 | | |
| 46 | NM_006430 | chaperonin containing | 0.067 | | | |
| | | TCP1, subunit 4 (delta) | | | | |
| 47 | NM_006310 | aminopeptidase puromycin | 0.066 | | | |
| | | sensitive | | | | |
| 48 | NM_006325 | RAN, member RAS | 0.065 | | | |
| | | oncogene family | | | | |
| 50 | NM_005348 | Hsp90, alpha | 0.061 | | | |
| 51 | NM_007995 | ficolin A | 0.057 | | | |
| 52 | NM_019685 | RuvB-like protein 1 | 0.057 | | | |
| 53 | NM_004461 | phenylalanine-tRNA | 0.055 | | | |
| | | synthetase-like | | | | |
| 54 | NM_000917 | procollagen-proline, 2- | 0.054 | | | |
| | | oxoglutarate 4- | | | | |
| | | dioxygenase (proline 4- | | | | |
| | | hydroxylase), alpha | | | | |
| | | polypeptide I | | | | |
| 55 | NM_019942 | septin 6 | 0.049 | | | |
| 56 | NM_017314 | ubiquitin C | 0.046 | 0.027 | | |
| 57 | NM_004522 | kinesin family member 5C | 0.04 | | | |
| 58 | NM_007508 | ATPase, H+ transporting, | 0.039 | | | |
| | | V1 subunit A, isoform 1 | | | | |
| 59 | NM_030706 | tripartite motif protein 2 | 0.039 | | | |
| 60 | NM_009955 | dihydropyrimidinase-like 2 | 0.037 | | | |
| 61 | NM_032069 | Glutamate receptor | 0.036 | | | |
| | | interacting protein | | | | |
| 62 | NM_002155 | Hsp70B′ | 0.034 | 0.061 | 0.107 | 0.034 |
| 63 | NM_003794 | sorting nexin 4 | 0.033 | | 0.102 | |
| 64 | NM_033309 | hypothetical protein | 0.032 | 0.032 | | |
| | | MGC4655 | | | | |
| 65 | NM_016338 | importin 11 | 0.032 | | | |
| 66 | NM_004521 | kinesin family member 5B | 0.028 | | | |
| 67 | NM_007054 | kinesin family member 3A | 0.027 | | | |
| 68 | NM_008633 | microtubule-associated | 0.025 | | | |
| | | protein 4 | | | | |
| 69 | NM_023115 | protocadherin 15 | 0.024 | | | |
| 70 | NM_016448 | RA-regulated nuclear | 0.015 | | | |
| | | matrix-associated protein | | | | |
| 71 | NM_000038 | adenomatosis polyposis | 0.014 | | | |
| | | coli | | | | |
| 72 | NM_004624 | vasoactive intestinal | 0.014 | | | |
| | | peptide receptor 1 | | | | |
| 73 | NM_004652 | ubiquitin specific protease | 0.014 | | | |
| | | 9, X chromosome (fat | | | | |
| | | facets-like Drosophila) | | | | |
| 74 | NM_022954 | MEGF1 | 0.012 | | | |
| 75 | NM_000296 | polycystic kidney disease 1 | 0.009 | | | |
| | | (autosomal dominant) | | | | |
| 76 | NM_000100 | cystatin B (stefin B) | | 0.337 | | |
| 77 | NM_007108 | transcription elongation | | 0.314 | 0.398 | 0.314 |
| | | factor B (SIll), polypeptide | | | | |
| | | 2 (18 kDa, elongin B) | | | | |
| 78 | NM_002965 | S100 calcium binding | | 0.246 | | |
| | | protein A9 (calgranulin B) | | | | |
| 79 | NM_008143 | guanine nucleotide binding | | 0.243 | | |
| | | protein, beta 2, related | | | | |
| | | sequence 1 | | | | |
| 80 | NM_007355 | heat shock 90 kDa protein | | 0.229 | | |
| | | 1, beta | | | | |
| 81 | NM_002818 | proteasome (prosome, | | 0.226 | | |
| | | macropain) activator | | | | |
| | | subunit 2 (PA28 beta) | | | | |
| 82 | NM_002306 | lectin, galactoside-binding, | | 0.212 | | |
| | | soluble, 3 (galectin 3) | | | | |
| 83 | NM_017147 | cofilin 1 | | 0.205 | | |
| 84 | NM_002963 | S100 calcium binding | | 0.198 | 0.198 | |
| | | protein A7 (psoriasin 1) | | | | |
| 85 | NM_006070 | TRK-fused gene | | 0.195 | | |
| 86 | NM_016647 | mesenchymal stem cell | | 0.178 | 0.178 | |
| | | protein DSCD75 | | | | |
| 87 | NM_021199 | sulfide quinone reductase- | | 0.151 | 0.073 | 0.151 |
| | | like (yeast) | | | | |
| 88 | NM_004785 | NHERF-2 | | 0.139 | 0.198 | |
| 89 | NM_002156 | heat shock 60 kDa protein | | 0.133 | | |
| | | 1 (chaperonin) | | | | |
| 90 | NM_005527 | heat shock 70 kDa protein | | 0.131 | 0.109 | |
| | | 1-like | | | | |
| 91 | NM_014225 | protein phosphatase 2 | | 0.126 | | |
| | | (formerly 2A), regulatory | | | | |
| | | subunit A (PR 65), alpha | | | | |
| | | isoform | | | | |
| 92 | NM_012111 | Aha1, activator of heat | 0.34 | 0.121 | | |
| | | shock 90 kDa protein | | | | |
| | | ATPase homolog 1 (yeast) | | | | |
| 93 | NM_006415 | serine | | 0.116 | | |
| | | palmitoyltransferase, long | | | | |
| | | chain base subunit 1 | | | | |
| 95 | NM_004208 | programmed cell death 8 | 0.093 | | | |
| | | (apoptosis-inducing factor) | | | | |
| 96 | NM_022934 | DnaJ-like protein | 0.081 | 0.081 | | |
| 97 | NM_021863 | testis-specific heat shock | 0.062 | 0.103 | 0.079 | |
| | | protein-related gene hst70 | | | | |
| 98 | NM_019390 | lamin A | 0.058 | 0.162 | | |
| 99 | NM_000462 | ubiquitin protein ligase | 0.055 | | | |
| | | E3A (human papilloma | | | | |
| | | virus E6-associated | | | | |
| | | protein, Angelman | | | | |
| | | syndrome) | | | | |
| 100 | NM_002808 | proteasome (prosome, | 0.04 | | | |
| | | macropain) 26S subunit, | | | | |
| | | non-ATPase, 2 | | | | |
| 101 | NM_024334 | hypothetical protein | 0.022 | | | |
| | | MGC3222 | | | | |
| 102 | NM_004327 | breakpoint cluster region | 0.017 | | | |
| 103 | NM_001035 | ryanodine receptor 2 | 0.006 | | | |
| | | (cardiac) | | | | |
| 104 | NM_005648 | transcription elongation | | 0.357 | | |
| | | factor B (SIII), polypeptide | | | | |
| | | 1 (15 kDa, elongin C) | | | | |
| 106 | NM_005389 | protein-L-isoaspartate (D- | | 0.33 | 0.617 | |
| | | aspartate) O- | | | | |
| | | methyltransferase | | | | |
| 107 | NM_008786 | protein-L-isoaspartate (D- | | 0.291 | | |
| | | aspartate) O- | | | | |
| | | methyltransferase 1 | | | | |
| 108 | NM_013232 | programmed cell death 6 | | 0.215 | | |
| 109 | NM_010481 | GRP75 | | 0.189 | 0.262 | |
| 110 | NM_000117 | emerin (Emery-Dreifuss | | 0.181 | | |
| | | muscular dystrophy) | | | | |
| 112 | NM_018144 | likely ortholog of mouse | | 0.113 | | |
| | | SEC61, alpha subunit 2 (S. cerevisiae) | | | | |
| 114 | NM_009795 | calpain, small subunit 1 | | 0.108 | 0.134 | |
| 115 | NM_007126 | valosin-containing protein | | 0.096 | | |
| 116 | NM_030971 | similar to rat tricarboxylate | | 0.093 | | |
| | | carrier-like protein | | | | |
| 117 | NM_022314 | tropomyosin 3, gamma | | 0.085 | 0.349 | |
| 118 | NM_006149 | lectin, galactoside-binding, | | 0.056 | 0.053 | |
| | | soluble, 4 (galectin 4) | | | | |
| 119 | NM_014612 | chromosome 9 open | | 0.051 | | |
| | | reading frame 10 | | | | |
| 120 | NM_016451 | coatomer protein complex, | | 0.051 | | |
| | | subunit beta | | | | |
| 121 | NM_005358 | LIM domain only 7 | | 0.05 | | |
| 122 | NM_013245 | vacuolar protein sorting 4A | | 0.046 | | |
| | | (yeast) | | | | |
| 123 | NM_016739 | GPI-anchored membrane | | 0.046 | | |
| | | protein 1 | | | | |
| 124 | NM_007245 | ataxin 2 related protein | | 0.044 | | |
| 125 | NM_004238 | thyroid hormone receptor | | 0.015 | | |
| | | interactor 12 | | | | |
| 126 | NM_006904 | protein kinase, DNA- | | 0.014 | 0.013 | |
| | | activated, catalytic | | | | |
| | | polypeptide | | | | |
| 127 | NM_031819 | FAT tumor suppressor | | 0.004 | | |
| | | (Drosophila) homolog | | | | |
| 128 | NM_001540 | heat shock 27 kDa protein 1 | | | 0.346 | |
| 129 | NM_013474 | apolipoprotein A-II | | | 0.324 | |
| 130 | NM_000611 | CD59 antigen p18-20 | | | 0.297 | |
| | | (antigen identified by | | | | |
| | | monoclonal antibodies | | | | |
| | | 16.3A5, EJ16, EJ30, EL32 | | | | |
| | | and G344) | | | | |
| 131 | NM_031469 | SH3 domain binding | | | 0.29 | |
| | | glutamic acid-rich protein | | | | |
| | | like 2 | | | | |
| 132 | NM_023009 | MARCKS-like protein | | | 0.251 | |
| 133 | NM_018362 | lin-7 homolog C (C. elegans) | | | 0.228 | |
| 134 | NM_006118 | HS1 binding protein | | | 0.201 | |
| 135 | NM_014666 | enthoprotin | | | 0.174 | |
| 136 | NM_023945 | membrane-spanning 4- | | | 0.17 | |
| | | domains, subfamily A, | | | | |
| | | member 5 | | | | |
| 137 | NM_002067 | guanine nucleotide binding | | | 0.162 | |
| | | protein (G protein), alpha | | | | |
| | | 11 (Gq class) | | | | |
| 138 | NM_001833 | clathrin, light polypeptide | | | 0.138 | |
| | | (Lca) | | | | |
| 139 | NM_002354 | tumor-associated calcium | | | 0.131 | |
| | | signal transducer 1 | | | | |
| 140 | NM_018188 | hypothetical protein | | | 0.128 | |
| | | FLJ10709 | | | | |
| 141 | NM_017724 | leucine rich repeat (in FLII) | | | 0.118 | |
| | | interacting protein 2 | | | | |
| 142 | NM_016963 | tropomodulin 3 | | | 0.116 | |
| 143 | NM_031033 | guanine nucleotide-binding | | | 0.111 | |
| | | protein alpha 11 subunit | | | | |
| 144 | NM_004447 | epidermal growth factor | | | 0.101 | |
| | | receptor pathway substrate 8 | | | | |
| 145 | NM_002070 | guanine nucleotide binding | | | 0.082 | |
| | | protein (G protein), alpha | | | | |
| | | inhibiting activity | | | | |
| | | polypeptide 2 | | | | |
| 146 | NM_001835 | clathrin, heavy | | | 0.077 | |
| | | polypeptide-like 1 | | | | |
| 147 | NM_002087 | granulin | | | 0.074 | |
| 148 | NM_019653 | WD-40-repeat-containing | | | 0.074 | |
| | | protein with a SOCS box 1 | | | | |
| 149 | NM_009386 | tight junction protein 1 | | | 0.073 | |
| 150 | NM_002778 | prosaposin (variant | | | 0.071 | |
| | | Gaucher disease and | | | | |
| | | variant metachromatic | | | | |
| | | leukodystrophy) | | | | |
| 151 | NM_004360 | cadherin 1, type 1, E- | | | 0.068 | |
| | | cadherin (epithelial) | | | | |
| 152 | NM_031922 | Reps1 | | | 0.062 | |
| 153 | NM_014935 | phosphoinositol 3- | | | 0.06 | |
| | | phosphate-binding protein-3 | | | | |
| 154 | NM_022098 | hypothetical protein | | | 0.055 | |
| | | LOC63929 | | | | |
| 155 | NM_001343 | Dab2 | | | 0.053 | |
| 156 | NM_004475 | flotillin 2 | | | 0.05 | |
| 157 | NM_016224 | sorting nexin 9 | | | 0.05 | |
| 158 | NM_014271 | interleukin 1 receptor | | | 0.049 | |
| | | accessory protein-like 1 | | | | |
| 159 | NM_014812 | KARP-1-binding protein | | | 0.049 | |
| 160 | NM_002958 | RYK receptor-like tyrosine | | | 0.048 | |
| | | kinase | | | | |
| 161 | NM_033299 | phospholipase D gene 2 | | | 0.045 | |
| 162 | NM_023063 | epithelial protein lost in | | | 0.044 | |
| | | neoplasm | | | | |
| 163 | NM_014428 | tight junction protein 3 | | | 0.039 | |
| | | (zona occludens 3) | | | | |
| 164 | NM_031382 | testis expressed gene 16 | | | 0.037 | |
| 165 | NM_033049 | mucin 13, epithelial | | | 0.037 | |
| | | transmembrane | | | | |
| 166 | NM_016745 | ATPase, Ca++ | | | 0.032 | |
| | | transporting, ubiquitous | | | | |
| 167 | NM_031823 | Wolfram syndrome 1 | | | 0.027 | |
| 168 | NM_001115 | adenylate cyclase 8 (brain) | | | 0.024 | |
| 169 | NM_007454 | AP-1, beta 1 subunit | | | 0.024 | |
| 170 | NM_001285 | CLCA1 | | | 0.023 | |
| 171 | NM_003253 | T-cell lymphoma invasion | | | 0.023 | |
| | | and metastasis 1 | | | | |
| 172 | NM_003174 | supervillin | | | 0.019 | |
| 173 | NM_015756 | shroom | | | 0.018 | |
| 174 | NM_003105 | SORL1 | | | 0.01 | |

TABLE 8
Comparison of wild-type and ΔF508 CFTR BHK proteome

| | | | Sequence | |
|---|---|---|---|---|
| | | | Coverage (%) | |
| | | | BHK | |
| Protein # | Refseq AC | gene name | ΔF508 | BHK wt |
| 1 | NM_000492 | cystic fibrosis transmembrane | 0.569 | 0.599 |
| | | conductance regulator | | |
| 2 | NM_008379 | karyopherin (importin) beta 1 | 0.209 | 0.579 |
| 3 | NM_009037 | reticulocalbin | 0.274 | 0.495 |
| 4 | NM_006597 | Hsc70 | 0.582 | 0.466 |
| 5 | NM_001539 | Hsp40-A1 (Hdj2) | 0.307 | 0.403 |
| 6 | NM_006391 | importin 7 | 0.043 | 0.334 |
| 7 | NM_022310 | GRP78 | 0.397 | 0.328 |
| 8 | NM_005507 | cofilin 1 (non-muscle) | 0.337 | 0.307 |
| 9 | NM_005880 | Hsp40-A2 (Hdj3) | 0.318 | 0.282 |
| 10 | NM_002715 | protein phosphatase 2 (formerly 2A), | 0.084 | 0.275 |
| | | catalytic subunit, alpha isoform | | |
| 11 | NM_001316 | CSE1 chromosome segregation 1-like | 0.045 | 0.255 |
| | | (yeast) | | |
| 12 | NM_002717 | protein phosphatase 2 (formerly 2A), | 0.235 | 0.248 |
| | | regulatory subunit B (PR 52), alpha | | |
| | | isoform | | |
| 13 | NM_023565 | chromosome segregation 1-like (S. cerevisiae) | 0.045 | 0.238 |
| 14 | NM_011992 | reticulocalbin 2 | 0.087 | 0.221 |
| 15 | NM_002901 | reticulocalbin 1, EF-hand calcium | 0.224 | 0.215 |
| | | binding domain | | |
| 16 | NM_008302 | Hsp90, beta | 0.358 | 0.214 |
| 17 | NM_012030 | NHERF-1 | 0.152 | 0.197 |
| 18 | NM_006098 | guanine nucleotide binding protein (G | | 0.189 |
| | | protein), beta polypeptide 2-like 1 | | |
| 19 | NM_002902 | reticulocalbin 2, EF-hand calcium | | 0.183 |
| | | binding domain | | |
| 20 | NM_011313 | S100 calcium binding protein A6 | | 0.18 |
| | | (calcyclin) | | |
| 22 | NM_018243 | hypothetical protein FLJ10849 | 0.184 | 0.175 |
| 23 | NM_009906 | ceroid-lipofuscinosis, neuronal 2 | | 0.16 |
| 24 | NM_002882 | RAN binding protein 1 | | 0.159 |
| 25 | NM_007479 | ADP-ribosylation factor 4 | | 0.156 |
| 26 | NM_003400 | exportin 1 (CRM1 homolog, yeast) | | 0.154 |
| 27 | NM_002716 | protein phosphatase 2 (formerly 2A), | 0.201 | 0.151 |
| | | regulatory subunit A (PR 65), beta | | |
| | | isoform | | |
| 28 | NM_001681 | SERCA2 | 0.085 | 0.15 |
| 29 | NM_021594 | ERM-binding phosphoprotein | 0.104 | 0.149 |
| 30 | NM_005998 | chaperonin containing TCP1, subunit | | 0.14 |
| | | 3 (gamma) | | |
| 31 | NM_007637 | chaperonin subunit 5 (epsilon) | | 0.131 |
| 32 | NM_011664 | ubiquitin B | 0.161 | 0.121 |
| 33 | NM_021671 | db83 | | 0.117 |
| 34 | NM_013336 | protein transport protein SEC61 alpha | | 0.111 |
| | | subunit isoform 1 | | |
| 35 | NM_028152 | MMS19 (MET18 S. cerevisiae)-like | | 0.11 |
| 36 | NM_004282 | BAG-2 | 0.28 | 0.104 |
| 37 | NM_001746 | calnexin | 0.164 | 0.096 |
| 38 | NM_012470 | transportin-SR | | 0.094 |
| 39 | NM_025291 | steroid receptor RNA activator 1 | | 0.091 |
| 40 | NM_013686 | TCP1 | 0.095 | 0.09 |
| 41 | NM_005345 | Hsp70-1A | 0.193 | 0.083 |
| 42 | NM_020645 | chromosome 11 open reading frame | | 0.083 |
| | | 14 | | |
| 43 | NM_018307 | ras homolog gene family, member T1 | | 0.081 |
| 44 | NM_021979 | Hsp70-2 | 0.156 | 0.078 |
| 45 | NM_001219 | calumenin | | 0.07 |
| 46 | NM_006430 | chaperonin containing TCP1, subunit | | 0.067 |
| | | 4 (delta) | | |
| 47 | NM_006310 | aminopeptidase puromycin sensitive | | 0.066 |
| 48 | NM_006325 | RAN, member RAS oncogene family | | 0.065 |
| 50 | NM_005348 | Hsp90, alpha | 0.392 | 0.061 |
| 51 | NM_007995 | ficolin A | | 0.057 |
| 52 | NM_019685 | RuvB-like protein 1 | 0.143 | 0.057 |
| 53 | NM_004461 | phenylalanine-tRNA synthetase-like | | 0.055 |
| 54 | NM_000917 | procollagen-proline, 2-oxoglutarate 4- | | 0.054 |
| | | dioxygenase (proline 4-hydroxylase), | | |
| | | alpha polypeptide I | | |
| 55 | NM_019942 | septin 6 | | 0.049 |
| 56 | NM_017314 | ubiquitin C | 0.06 | 0.046 |
| 57 | NM_004522 | kinesin family member 5C | 0.061 | 0.04 |
| 58 | NM_007508 | ATPase, H+ transporting, V1 subunit | | 0.039 |
| | | A, isoform 1 | | |
| 59 | NM_030706 | tripartite motif protein 2 | | 0.039 |
| 60 | NM_009955 | dihydropyrimidinase-like 2 | | 0.037 |
| 61 | NM_032069 | Glutamate receptor interacting protein | | 0.036 |
| 62 | NM_002155 | Hsp70B′ | 0.096 | 0.034 |
| 63 | NM_003794 | sorting nexin 4 | | 0.033 |
| 64 | NM_033309 | hypothetical protein MGC4655 | 0.032 | 0.032 |
| 65 | NM_016338 | importin 11 | 0.045 | 0.032 |
| 66 | NM_004521 | kinesin family member 5B | 0.038 | 0.028 |
| 67 | NM_007054 | kinesin family member 3A | | 0.027 |
| 68 | NM_008633 | microtubule-associated protein 4 | | 0.025 |
| 69 | NM_023115 | protocadherin 15 | | 0.024 |
| 70 | NM_016448 | RA-regulated nuclear matrix- | 0.015 | 0.015 |
| | | associated protein | | |
| 71 | NM_000038 | adenomatosis polyposis coli | 0.013 | 0.014 |
| 72 | NM_004624 | vasoactive intestinal peptide receptor 1 | | 0.014 |
| 73 | NM_004652 | ubiquitin specific protease 9, X | | 0.014 |
| | | chromosome (fat facets-like | | |
| | | Drosophila) | | |
| 74 | NM_022954 | MEGF1 | | 0.012 |
| 75 | NM_000296 | polycystic kidney disease 1 | 0.031 | 0.009 |
| | | (autosomal dominant) | | |
| 76 | NM_014225 | protein phosphatase 2 (formerly 2A), | 0.413 | |
| | | regulatory subunit A (PR 65), alpha | | |
| | | isoform | | |
| 78 | NM_022934 | DnaJ-like protein | 0.34 | |
| 79 | NM_010481 | GRP75 | 0.337 | |
| 80 | NM_007175 | chromosome 8 open reading frame 2 | 0.324 | |
| 81 | NM_002965 | S100 calcium binding protein A9 | 0.263 | |
| | | (calgranulin B) | | |
| 82 | NM_007126 | valosin-containing protein | 0.246 | |
| 83 | NM_002795 | proteasome (prosome, macropain) | 0.239 | |
| | | subunit, beta type, 3 | | |
| 84 | NM_005866 | type I sigma receptor | 0.229 | |
| 85 | NM_011185 | proteasome (prosome, macropain) | 0.229 | |
| | | subunit, beta type 1 | | |
| 86 | NM_002793 | proteasome (prosome, macropain) | 0.228 | |
| | | subunit, beta type, 1 | | |
| 87 | NM_011967 | proteasome (prosome, macropain) | 0.228 | |
| | | subunit, alpha type 5 | | |
| 88 | NM_006459 | similar to Caenorhabditis elegans | 0.171 | |
| | | protein C42C1.9 | | |
| 89 | NM_008944 | proteasome (prosome, macropain) | 0.171 | |
| | | subunit, alpha type 2 | | |
| 90 | NM_007688 | cofilin 2, muscle | 0.169 | |
| 91 | NM_010223 | FKBP8 | 0.166 | |
| 92 | NM_011971 | proteasome (prosome, macropain) | 0.166 | |
| | | subunit, beta type 3 | | |
| 93 | NM_024661 | hypothetical protein FLJ12436 | 0.162 | |
| 94 | NM_002796 | proteasome (prosome, macropain) | 0.159 | |
| | | subunit, beta type, 4 | | |
| 95 | NM_006601 | p23 | 0.156 | |
| 96 | NM_019766 | telomerase binding protein, p23 | 0.156 | |
| 97 | NM_025736 | RIKEN cDNA 4921531G14 gene | 0.145 | |
| 98 | NM_008143 | guanine nucleotide binding protein, | 0.142 | |
| | | beta 2, related sequence 1 | | |
| 99 | NM_000942 | cyclophilin B | 0.13 | |
| 100 | NM_002808 | proteasome (prosome, macropain) | 0.129 | |
| | | 26S subunit, non-ATPase, 2 | | |
| 101 | NM_006503 | proteasome (prosome, macropain) | 0.124 | |
| | | 26S subunit, ATPase, 4 | | |
| 102 | NM_013863 | BAG-3 | 0.121 | |
| 103 | NM_016737 | Hop | 0.114 | |
| 104 | NM_013559 | Hsp105 | 0.095 | |
| 105 | NM_016127 | hypothetical protein MGC8721 | 0.088 | |
| 106 | NM_017374 | protein phosphatase 2a, catalytic | 0.084 | |
| | | subunit, beta isoform | | |
| 107 | NM_011889 | septin 3 | 0.082 | |
| 108 | NM_014673 | KIAA0103 gene product | 0.081 | |
| 110 | NM_016742 | Cdc37 | 0.079 | |
| 111 | NM_008948 | proteasome (prosome, macropain) | 0.077 | |
| | | 26S subunit, ATPase 3 | | |
| 112 | NM_018085 | importin 9 | 0.077 | |
| 113 | NM_025754 | RIKEN cDNA 4933425L11 gene | 0.074 | |
| 114 | NM_018448 | TBP-interacting protein | 0.072 | |
| 115 | NM_002271 | karyopherin (importin) beta 3 | 0.063 | |
| 116 | NM_004576 | protein phosphatase 2 (formerly 2A), | 0.063 | |
| | | regulatory subunit B (PR 52), beta | | |
| | | isoform | | |
| 117 | NM_015129 | septin 6 | 0.062 | |
| 118 | NM_011304 | RuvB-like protein 2 | 0.06 | |
| 119 | NM_016395 | butyrate-induced transcript 1 | 0.056 | |
| 120 | NM_009864 | cadherin 1 | 0.055 | |
| 121 | NM_015292 | likely ortholog of mouse membrane | 0.053 | |
| | | bound C2 domain containing protein | | |
| 122 | NM_002815 | proteasome (prosome, macropain) | 0.05 | |
| | | 26S subunit, non-ATPase, 11 | | |
| 123 | NM_018695 | erbb2 interacting protein | 0.048 | |
| 124 | NM_008803 | phosphodiesterase 8A | 0.045 | |
| 125 | NM_004734 | doublecortin and CaM kinase-like 1 | 0.044 | |
| 126 | NM_008450 | kinesin 2 | 0.044 | |
| 127 | NM_006640 | MLL septin-like fusion | 0.042 | |
| 128 | NM_031508 | Glutamate receptor, ionotropic, | 0.042 | |
| | | kainate 5 | | |
| 129 | NM_019548 | trophinin | 0.041 | |
| 130 | NM_004320 | SERCA1 | 0.033 | |
| 131 | NM_017249 | membrane bound C2 domain | 0.026 | |
| | | containing protein | | |
| 132 | NM_000014 | alpha-2-macroglobulin | 0.023 | |
| 133 | NM_019120 | protocadherin beta 8 | 0.022 | |
| 134 | NM_004274 | A kinase (PRKA) anchor protein 6 | 0.019 | |
| 135 | NM_005120 | trinucleotide repeat containing 11 | 0.018 | |
| | | (THR-associated protein, 230 kDa | | |
| | | subunit) | | |
| 136 | NM_019226 | dynein, cytoplasmic, heavy chain 1 | 0.015 | |
| 137 | NM_031819 | FAT tumor suppressor (Drosophila) | 0.008 | |
| | | homolog | | |
| 138 | NM_001036 | ryanodine receptor 3 | 0.005 | |
| 139 | NM_012111 | Aha1, activator of heat shock 90 kDa | 0.15 | 0.34 |
| | | protein ATPase homolog 1 (yeast) | | |

### Example 2

### CFTR Spectra Linkage

From the wealth of interactions observed in the interactome (see Example 1), the basis for the loss of export of ΔF508 from the ER was examined as a means of understanding the most common form of CF. A change in protein folding energetics (Sekijima et al., 2005, Cell 121, 73-85; Strickland and Thomas, 1997, J Biol Chem 272, 25421-25424) in response to the Phe 508 deletion results in failure of ΔF508 CFTR to couple to the COPII budding machinery (Wang et al., 1998, FEBS Lett 427, 103), resulting in ER-associated degradation (ERAD) (Nishikawa 2005, J Biochem (Tokyo) 137, 551). Chaperone components that are currently thought to significantly affect CFTR folding through ERAD (Sekijima et al., 2005, supra) pathways include calnexin (Farinha and Amaral, 2005, Mol Cell Biol 25, 5242; Okiyoneda et al., 2004, Mol Biol Cell 15, 563; Pind et al., 1994, J Biol Chem 269, 12784) found in the lumen of the ER, as well as the cytosolic chaperone complexes Hsc-Hsp70/40 and Hsp90 (Albert et al., 2004, Mol Biol Cell 15, 4003; Amaral, 2004, supra; Loo et al., 1998, Embo J 17, 6879; Meacham et al., 1999, Embo J 18, 1492; Meacham et al., 2001, Nat Cell Biol 3, 100; Strickland et al., 1997, J Biol Chem 272, 25421; Younger et al., 2004, supra).

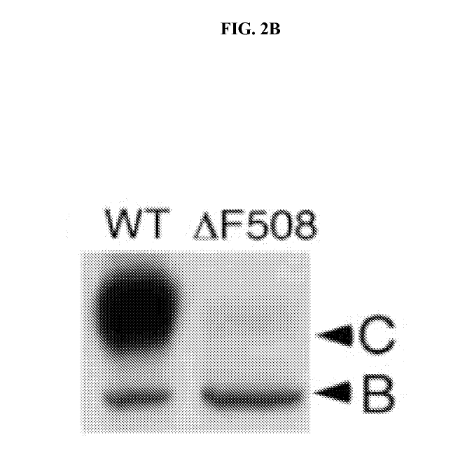

Consistent with these results, the proteomes of wild-type CFTR expressing cells (see e.g., FIG. 1) showed robust linkage based on total spectra recovered (see e.g., Table 7) to calnexin, Hsc-Hsp70/40 and Hsp90 cytosolic chaperones. These chaperone components likely define core machineries (FIG. 2) facilitating folding of wild-type CFTR as has been observed for other proteins (McClellan et al., 2005, Nat Cell Biol 7, 736-741).

Results showed that the proteomes of wild-type CFTR expressing cells (FIG. 1) showed robust linkage based on total spectra recovered (see e.g., Table 7) to calnexin, Hsc-Hsp70/40 and Hsp90 cytosolic chaperones. These results are consistent with reports that chaperone components currently thought to significantly affect CFTR folding through ERAF (Sekijima et al., 2005, supra) pathways include calnexin (Farinha and Amaral, 2005, supra; Okiyoneda et al., 2004, supra; Pind et al., 1994, J Biol Chem 269, 12784-12788) found in the lumen of the ER, as well as the cytosolic chaperone complexes Hsc-Hsp70/40 and Hsp90 (Albert et al., 2004, supra; Amaral, 2004, supra; Loo et al., 1998, supra; Meacham et al., 1999, supra; Meacham et al., 2001, supra; Strickland et al., 1997, supra; Younger et al., 2004, supra).

These chaperone components likely define core machineries (see e.g., FIG. 2A) facilitating folding of wild-type CFTR, as has been observed for other proteins (McClellan et al., 2005, supra).

TABLE 9
CFTR ER-associated folding proteome*

| | | ΔF508 CFTR | | | wt CFTR | | |
|---|---|---|---|---|---|---|---|
| | | | | | % | | |
| | | % sequence | | | sequence | | |
| | | coverage | unique total | | coverage | unique total | |
| RefSeq AC | Protein Name | A | B | C | A | B | C |
| NM_000492 | CFTR | 57 | 229 | 2481 | 60 | 333 | 4172 |
| NM_024351 | Hsc70 | 60 | 66 | 369 | 48 | 39 | 132 |
| NM_022310 | GRP78^{#} | 40 | 30 | 85 | 33 | 17 | 37 |
| NM_021979 | Hsp70-2 | 16 | 23 | 57 | 8 | 7 | 17 |
| NM_001746 | Calnexin^{#} | 16 | 13 | 52 | 10 | 4 | 7 |
| NM_008302 | Hsp90β | 36 | 24 | 49 | 21 | 11 | 18 |
| NM_010481 | GRP75 | 34 | 23 | 40 | | | |
| NM_005348 | Hsp90□ | 39 | 24 | 37 | 6 | 4 | 5 |
| NM_022934 | DnaJ-like | 34 | 11 | 35 | | | |
| | protein | | | | | | |
| NM_001539 | Hsp40-A1 | 31 | 10 | 33 | 40 | 13 | 25 |
| | (Hdj2) | | | | | | |
| NM_005345 | Hsp70-1A | 19 | 12 | 20 | 8 | 4 | 6 |
| NM_004282 | BAG-2 | 28 | 7 | 20 | 10 | 2 | 2 |
| NM_005880 | Hsp40-A2 | 32 | 9 | 19 | 28 | 6 | 16 |
| | (Hdj3) | | | | | | |
| NM_002155 | Hsp70B′ | 10 | 8 | 14 | 3 | 3 | 5 |
| NM_013559 | Hsp105 | 10 | 5 | 6 | | | |
| NM_013686 | TCP1 | 10 | 3 | 5 | 9 | 3 | 5 |
| NM_010223 | FKBP38 | 17 | 4 | 5 | | | |
| NM_013863 | BAG-3 | 12 | 4 | 5 | | | |
| NM_016737 | Hop | 11 | 4 | 4 | | | |
| NM_016742 | Cdc37 | 8 | 2 | 3 | | | |
| NM_000942 | cyclophilin B^{#} | 13 | 2 | 2 | | | |
| NM_006601 | p23 | 16 | 2 | 2 | | | |
| NM_012111 | Aha1 | 15 | 7 | 15 | 34 | 10 | 20 |
| NM_009037 | Reticulocalbin^{#} | 27 | 5 | 8 | 50 | 14 | 19 |
| NM_011992 | Reticulocalbin | 9 | 3 | 4 | 22 | 6 | 12 |
| | 2^{#} | | | | | | |
| NM_001219 | Calumenin^{#} | | | | 7 | 3 | 3 |

*Indicated are the interacting proteins in BHK cells, their percentage sequence coverage (A), number of unique spectra (B) and number of total spectra (C) as detected by mass spectrometry in cell lines examined (FIG. 1).
^{#}ER luminal chaperones.

### Example 3

### CFTR and ΔF508 Localization and Interactions

To identify components in the interactome that may be involved in the failure of ΔF508 to couple to the ER export machinery, the proteomes of wild-type and ΔF508 CFTR immunoprecipitated from BHK cells were compared. The parent BHK cell line not expressing CFTR was used as a negative control for non-specific interactions (see e.g., FIG. 2).

FIG. 2 is a series of depictions of the ER folding network. Table 8 shows the results of an array of proteins recovered using MudPIT in BHK cells not expressing CFTR (control), or those expressing either ΔF508 or wild-type CFTR, arranged in the order of fractional sequence coverage by mass spectrometry.

FIG. 2A is a cartoon depicting a composite view of the network comprising the CFTR ER folding and degradation proteomes. Light gray edges indicate potential direct or indirect interactions with CFTR; dark edges indicate known physical interactions between components based on data from HPRD, BIND, and IntAct protein interaction databases. Light green circle indicates core folding chaperones; the light pink circle indicates regulatory co-chaperones and ERAD components. FIG. 2B is an image of an SDS-PAGE immunoblot showing the typical steady-state levels of bands B and C observed in wild-type and ΔF508 CFTR expressing cells. For further methodology information, see Example 1.

For SDS-PAGE and immunoblotting, cells were washed twice with 500 μl of ice cold PBS and lysed by addition of 45 μl of freshly prepared TBS (50 mM Tris-HCl pH 7.0, 150 mM NaCl) supplemented with 1% Triton X-100 and protease inhibitor cocktail (Pierce) at 2 mg/ml of lysis buffer and incubated on ice for 30 min with occasional agitation. The lysates were collected and spun at 16,000×g for 20 min at 4° C. and the supernatants were collected and analyzed for protein concentration. The lysates (25 μg of total protein per lane) were separated by SDS-PAGE and transferred to nitrocellulose for Western blot analysis. Immunoblotting for actin (Chemicon, Temecula, Calif.) was used as an additional internal control for consistency of sample loading (not shown). CFTR was detected with a monoclonal antibody (M3A7 ascites) against an epitope at the C-terminal end of the second nucleotide binding domain (Kartner et al., 1992). p23 was detected with p23 ascites (JJ3, Abcam, Cambridge, Mass.), HOP with a rabbit polyclonal serum, FKBP8 with a rabbit polyclonal serum, and Aha1 with a rabbit Aha1 polyclonal serum. Also used was a monoclonal antibody (P5D4) against the C-terminal cytoplasmic tail of the vesicular stomatitis virus glycoprotein (VSV-G). The amount of each protein of interest was quantified by densitometry using an Alphalnnotech Fluorochem SP (Alphalnnotech, San Leandro, Calif.). Experiments were conducted in triplicates, and mean and standard error of the mean determined using an unpaired two-tailed t-test.

Results showed that, at physiological temperature (37° C.), wild-type CFTR is principally (>80-90%) in the band C Golgi processed glycoform found at the cell surface, with the remaining CFTR detected in the band B ER-associated core glycosylated glycoform (see e.g., FIG. 2B). In contrast, in cells expressing ΔF508 at 37° C., generally only 5-20% of the protein (reflecting cell type and growth conditions) can be detected in band C due to significantly reduced stability and folding for export. In this case, the protein is largely restricted to the immature core glycosylated band B ER glycoform (see e.g., FIG. 2B) where it is targeted for ERAD (Jensen et al., 1995, Cell 83, 129-135; Ward and Kopito, 1994, J Biol Chem 269, 25710-25718; Ward et al., 1995, Cell 83, 121-127). In addition to components likely involved in ERAD (Table 6), the ER folding interactome (see e.g., FIG. 2A) revealed that ΔF508, like wild-type CFTR, showed strong interactions with lumenal calnexin and the cytosolic Hsc-Hsp70/40 and Hsp90 cytosolic components (see e.g., Table 7). These results indicate that ΔF508 interacts with the core machinery directing the folding of wild-type CFTR (see e.g., FIG. 2A).

### Example 4

### Hsp90 Co-Chaperone Components in ΔF508 ER Interactome

The ΔF508 ER interactome was analyzed for the presence of Hsp90 co-chaperone components. Hsp90-dependent folding of a variety of client proteins is transiently regulated by co-chaperones (Picard, 2002, Cell Mol Life Sci 59, 1640-1648; Young et al., 2003, supra; Young et al., 2001, supra). Previous studies have suggested that folding of ΔF508 is kinetically impaired ((Qu et al., 1997, J Bioenerg Biomembr 29, 483-490; Qu et al., 1997, J Biol Chem 272, 15739-15744; Qu and Thomas, 1996, J Biol Chem 271, 7261-7264). Therefore, proteins found in the ΔF508 interactome would be expected to include those associated with folding intermediate(s) sensitive to the Phe 508 deletion that may accumulate in response to a kinetic defect in the folding pathway.

Results showed that a number of Hsp90 co-chaperone components in the ΔF508 ER interactome were not generally detected in the wild-type proteome (see e.g., FIG. 2A). This finding is consistent with the prediction of accumulated folding intermediate(s) sensitive to the Phe 508 deletion in ΔF508. Hsp90 co-chaperone components in the ΔF508 ER interactome that were not generally detected in the wild-type proteome included the Hsc-Hsp70/Hsp90 organizing protein (HOP), p23, Cdc37, the immunophilin FKBP8 and Aha1. Hsp90 co-chaperones have been studied for their roles as regulators of Hsp90-client interactions to modulate the fold of metastable client proteins including steroid hormone receptors (SHRs) and signaling kinases (Wegele, et al., 2004, supra). Other chaperone regulators detected included BAG-2/3 that have been studied for their role in regulation of Hsc-Hsp70 function in degradation of CFTR (Arndt et al., 2005, Mol Biol Cell; Dai et al., 2005, J Biol Chem 280, 37634), Hsp105, and the folding chaperonin TCP1.

The network of the known interactions between Hsc-Hsp70/40, Hsp90 and proteins potentially involved in their regulation illustrates the potential complexity of ΔF508 and wild-type CFTR folding pathways for ER export (see e.g., FIG. 2A).

### Example 5

### Effect of Co-Chaperone p23 on ΔF508 Folding in HEK293 Cells

The role of the key co-chaperone regulator p23 (Pratt and Toft, 2003, Exp Biol Med (Maywood) 228, 111-133; Prodromou and Pearl, 2003, Curr Cancer Drug Targets 3, 301-323; Wegele et al., 2004, supra) in HEK293 cells stably expressing ΔF508 was examined at several temperatures. P23, along with HOP and FKBP8, affect ATP-dependent folding steps in the cyclic Hsp90-client interaction pathway.

To begin to define the role of Hsp90 in folding and export of ΔF508 CFTR, dsRNA and transient transfection was used to control the level of protein expression of selected Hsp90 co-chaperones including p23 (see Example 5), HOP (see Example 6) and FKBP8 (see Example 7) that affect ATP-dependent folding steps in the cyclic Hsp90-client interaction pathway. Following recognition of a client molecule such as CFTR by the Hsc-Hsp70/40 complex, the ubiquitous co-chaperone HOP links the nascent Hsc-Hsp70/40-client complex to Hsp90 (Johnson et al., 1998, J Biol Chem 273, 3679-3686). Subsequently, the co-chaperone regulator p23, in the presence of ATP, displaces Hsc-Hsp70/40 and HOP to form the mature Hsp90-p23-client complex in the ATP-bound state (Wegele et al., 2004, supra). The cycling of Hsp90-client complexes containing p23 are regulated by immunophilins (Johnson and Toft, 1994, J Biol Chem 269, 24989-24993; Wu et al., 2004, Proc Natl Acad Sci USA 101, 8348-8353). Loss of the immunophilin FKBP52 in the case of the steroid hormone receptor (SHR), the prototypical Hsp90 client (Pratt and Toft, 2003, Exp Biol Med (Maywood) 228, 111-133; Prodromou and Pearl, 2003, supra), destabilizes the intermediate Hs 90-client chaperone complex, preventing hormone loading (Cheung-Flynn et al., 2005, Mol Endocrinol 19, 1654-1666).

HEK293 cells were maintained in DMEM supplemented with 10% FBS and Pen/Strep as above. HEK293 cells stably expressing ΔF508 CFTR were maintained in the same medium as above plus 150 μg/ml hygromycin B.

dsRNA and transient transfection were used to control the level of protein expression of p23. The cDNA clones for p23 was purchased from ATCC (Manassas, Va.) and were subcloned into pcDNA expression vector, and the sequence of the coding region was verified by DNA sequencing analysis. dsRNA solutions were prepared by mixing serum and antibiotic free DMEM or MEM-α with the indicated dsRNA at a working concentration of 0.6 μM (human p23, Ambion (Austin, Tex.) Cat. No. 16704 ID 18391) and 6 μl of HiPerFect (Qiagen, Valencia, Calif.) per well of a 12 well dish. Control dsRNA (Dharmacon Cat. No. CONJB-000015) was added at equal concentration to the dsRNA being tested. The dsRNA mixture (100 μl) was added to the cells containing 1.1 ml of the appropriate media at a final concentration of 50 nM and cultured at 37° C./5% CO₂ for 48 h. Upon completion of this incubation, the media was removed and replaced with 1.1 ml of fresh complete medium and 100 μl of freshly prepared dsRNA solution and cultured for an additional 33 h at 37° C./5% CO₂. Where indicated, the cells were subsequently transferred to a 30° C./5% CO₂ incubator or maintained at 37° C./5% CO₂, for an additional 15 h incubation.

Over-expression of human p23 was performed by co-transfecting HEK293 with plasmids expressing CFTR ΔF508 and p23 by vaccinia virus infection as previously described (Wang et al., 2004, supra). For samples analyzed at the permissive temperature cells were shifted to 30° C. for 15 h prior to harvesting.

SDS-PAGE and immunoblotting are as described in Example 3.

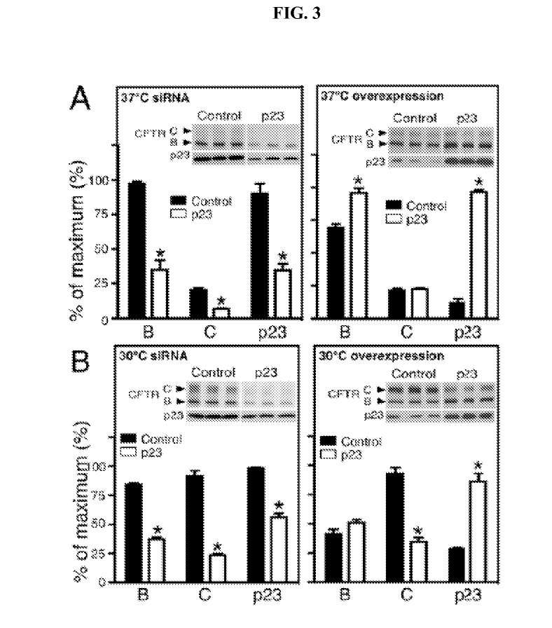

FIG. 3 is a series of bar graphs depicting the effect of the Hsp90 co-chaperone p23 on folding and export of ΔF508 from the ER. FIG. 3A is a pair of bar graphs showing percent maximum levels for the steady-state pool of ER glycoform ΔF508 (B), cell surface glycoform ΔF508 (C), and p23 expression. Human dsRNA to p23 (left panel) was used to reduce expression of the indicated protein at 37° C. Scrambled dsRNA was used as a control. Human cDNA to p23 (right panel) were used to overexpress the indicated protein at 37° C. The insets are images of an SDS-PAGE immunoblot for the steady-state pool of ER glycoform (band B) and cell surface glycoform (band C). The steady-state pools of bands B and C were determined using immunoblotting. FIG. 3B is as described for FIG. 3A except that cells were incubated at the permissive temperature (30°) to promote folding and export from the ER. The asterisks (*) indicate statistical significance (p≦0.05). Experiments were repeated independently in triplicate at least three times with representative results shown. For further methodology information, see Example 5.

Results showed that dsRNA reduction of p23 levels by ˜70% resulted in a comparable (60-70%) reduction in the steady-state pools of both the band B ER glycoform and the small pool of the band C cell surface glycoform when compared to the scrambled mock control (see e.g., FIG. 3A, left panel). Conversely, overexpression (3-5 fold) partially stabilized band B, but did not result in a significant increase in band C (see e.g., FIG. 3A, right panel). Interestingly, dsRNA reduction of p23 had a similar effect on stability of both band B and C wild-type CFTR in HEK293 (not shown), suggesting that p23 affects the dynamics of normal folding.

Because ΔF508 CFTR is a temperature sensitive folding mutant (Denning et al., 1992, Nature 358, 761-764), incubation of cells at the permissive temperature (30°) instead of 37° C. provides a more energetically favorable folding environment leading to significant levels of cell surface localized ΔF508.

Results showed that, at steady-state (15 h post temperature-shift from 37° C. to 30° C.), 40-50% of the total ΔF508 pool in HEK293 cells is typically found in band C (Denning et al., 1992, supra) (see e.g., FIG. 3B, left panel). Notably, even at the permissive folding temperature (30° C.), dsRNA reduction of p23 resulted in a significant decrease in the stability of band B and processing to band C (see e.g., FIG. 3B, left panel). At 30° C., overexpression had no effect on band B, but prevented processing to band C (see e.g., FIG. 3B, right panel). A similar dominant negative effect of p23 overexpression has been observed for other Hsp90-dependent signaling pathways reflecting excessive stabilization of the mature client complex (Pratt and Toft, 2003, supra).

Thus, consistent with the effects on wild-type CFTR, p23 is a modular component of folding that affects the stability of ER ΔF508 at both restrictive and permissive folding conditions. These results emphasize the potential differential role of the local chaperone environment on the kinetically impaired ΔF508 fold.

### Example 6

### Effect of Co-Chaperone FKBP8 on ΔF508 Folding in HEK293 Cells

The role of the co-chaperone regulator FKBP8 in HEK293 cells stably expressing ΔF508 was examined. Although unable to identify FKBP52 which is involved in SHR folding (Cheung-Flynn et al., 2005, supra) in the ΔF508 CFTR proteome, the immunophilin family member FKBP8 (Nielsen et al., 2001, Genomics 83, 181-192; Pedersen et al., 1999, Electrophoresis 20, 249-255) was detected. FKBP8 is a membrane-associated immunophilin that has been reported to be localized to both the mitochondria and the ER (Kang et al., 2005, FEBS Lett 579, 1469-1476; Shirane and Nakayama, 2003, Nat Cell Biol 5, 28-37; Weiwad et al., 2005, FEBS Lett 579, 1591-1596).

dsRNA and transient transfection were used to control the level of protein expression of FKBP8. The cDNA clones for FKBP8 were purchased from ATCC (Manassas, Va.) and were subcloned into pcDNA expression vector, and the sequence of the coding region was verified by DNA sequencing analysis. dsRNA solutions were prepared as in Example 5 but with human FKBP8, Ambion Cat. No. 16704 ID 45182. Over-expression of human FKBP8 was performed by co-transfecting HEK293 with plasmids expressing CFTR ΔF508 and FKBP8 by vaccinia virus infection as previously described (Wang et al., 2004, supra). For samples analyzed at the permissive temperature cells were shifted to 30° C. for 15 h prior to harvesting. SDS-PAGE and immunoblotting were as described in Example 3.

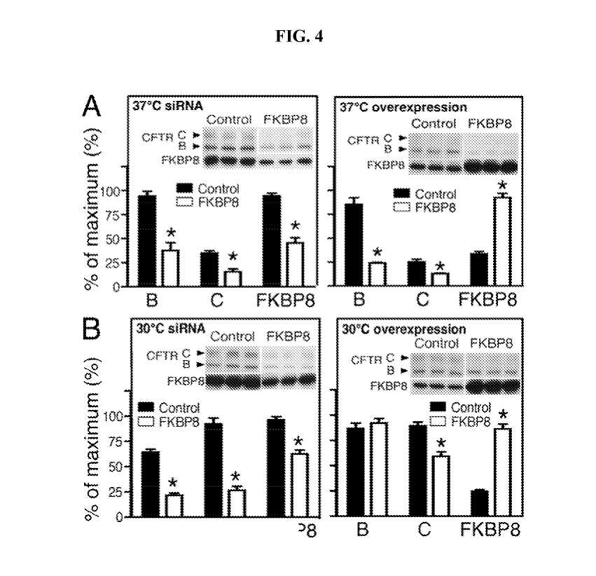

FIG. 4 is a series of bar graphs depicting the effect of the Hsp90 co-chaperone FKBP8 on folding and export of ΔF508 from the ER. FIG. 4A is a pair of bar graphs showing percent maximum levels for the steady-state pool of ER glycoform ΔF508 (B), cell surface glycoform ΔF508 (C), and FKBP8 expression. Human dsRNA to FKBP8 (left panel) was used to reduce expression of the indicated protein at 37° C. Scrambled dsRNA was used as a control. Human cDNA to FKBP8 (right panel) was used to overexpress the indicated protein at 37° C. The insets are images of an SDS-PAGE immunoblot for the steady-state pool of ER glycoform (band B) and cell surface glycoform (band C). FIG. 4B (B) is as described for FIG. 4A except that cells were incubated at the permissive temperature (30°) to promote folding and export from the ER. The asterisks (*) indicate statistical significance (p≦0.05). Experiments were repeated independently in triplicate at least three times with representative results shown. For further methodology information, see Example 6.

Results showed that FKBP8 has substantial overlap with the ER marker protein calnexin (not shown), a result consistent with previous reports. Similar to the effect of p23 dsRNA, significant destabilization of ΔF508 in response dsRNA reduction of FKBP8 at 37° C. was observed (see e.g., FIG. 4A, left panel). Interestingly, overexpression at 37° C. also destabilized CFTR (see e.g., FIG. 4A, right panel) raising the possibility that FKBP8 function is linked to the steady-state concentration of Hsp90. In contrast, dsRNA reduction of FKBP8 expression reduced (30-40%) the stability of ΔF508 CFTR at 30° C., with a corresponding reduction in the level of band C (−50%) (see e.g., FIG. 4B, left panel), whereas overexpression at 30° C. had only a modest effect on stability, yet it interfered with processing to band C (see e.g., FIG. 4B, right panel).

These results suggest that the Hsp90 co-chaperone FKBP8, like p23, acts as a folding modulator to control ΔF508 stability in the ER. These results emphasize the potential differential role of the local chaperone environment on the kinetically impaired ΔF508 fold.

### Example 7

### Effect of Co-Chaperone HOP on ΔF508 Folding in HEK293 Cells

The role of the co-chaperone regulator HOP in HEK293 cells stably expressing ΔF508 was examined. dsRNA and transient transfection were used to control the level of protein expression of HOP. The cDNA clones for HOP were purchased from ATCC (Manassas, Va.) and were subcloned into pcDNA expression vector, and the sequence of the coding region was verified by DNA sequencing analysis. dsRNA solutions were prepared as in Example 5 but with human HOP, Ambion Cat. No. 16704 ID 18719. Over-expression of human HOP was performed by co-transfecting HEK293 with plasmids expressing CFTR ΔF508 and HOP by vaccinia virus infection as previously described (Wang et al., 2004, supra). For samples analyzed at the permissive temperature cells were shifted to 30° C. for 15 h prior to harvesting. SDS-PAGE and immunoblotting were as described in Example 3.

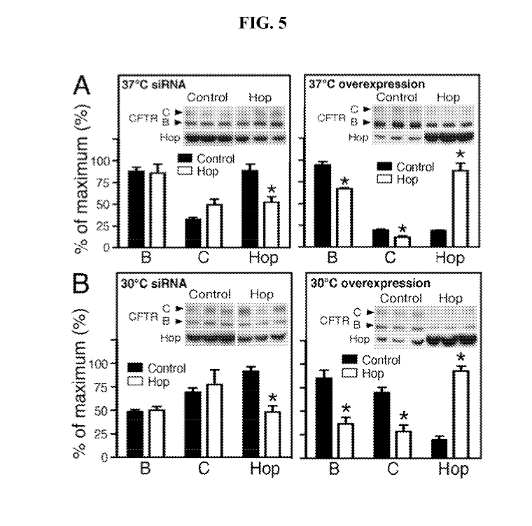

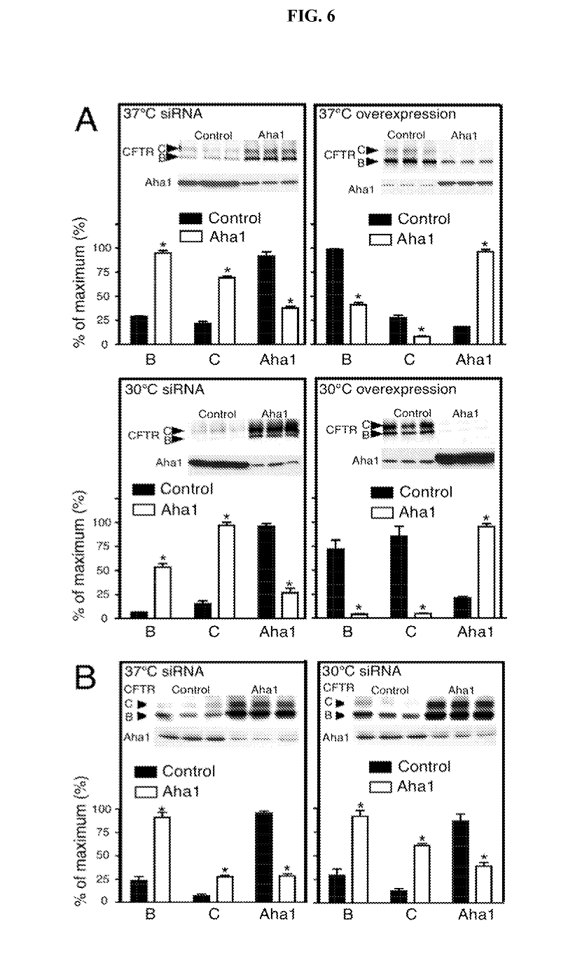

FIG. 5 is a series of bar graphs depicting the effect of the Hsp90 co-chaperone HOP on folding and export of ΔF508 from the ER. FIG. 5A is a pair of bar graphs showing percent maximum levels for the steady-state pool of ER glycoform ΔF508 (B), cell surface glycoform ΔF508 (C), and HOP expression. Human dsRNA to HOP (left panel) was used to reduce expression of the indicated protein at 37° C. Scrambled dsRNA was used as a control. Human cDNA to HOP (right panel) was used to overexpress the indicated protein at 37° C. The insets are images of an SDS-PAGE immunoblot for the steady-state pool of ER glycoform (band B) and cell surface glycoform (band C). FIG. 5B is as described for FIG. 5A except that cells were incubated at the permissive temperature (300) to promote folding and export from the ER. The asterisks (*) indicate statistical significance (p≦0.05). Experiments were repeated independently in triplicate at least three times with representative results shown. For further methodology information, see Example 7.

Results showed that, in contrast to the effects of dsRNA reduction of both p23 and FKBP8, the maximal reduction of HOP in response to dsRNA observed in HEK293 cells (40-60%) yielded little change in band B stability or the level of band C (see e.g., FIG. 5A, left panel), whereas overexpression (˜4-fold) partially destabilized both B and C (see e.g., FIG. 5A, right panel). Again, dsRNA of HOP did not effect folding or export of ΔF508 at 30° C. (see e.g., FIG. 5B, left panel). However, overexpression significantly destabilized the protein suggesting that a prolonged linkage to Hsc-Hsp 70/40 under permissive folding conditions favors targeting for degradation (see e.g., FIG. 5B, right panel).

These results suggest that that HOP facilitates a link between ΔF508 CFTR and Hsc-Hsp70/40 function in degradation (Arndt et al., 2005, supra; Meacham et al., 1999, supra; Meacham et al., 2001, supra; Younger et al., 2004, supra). These results emphasize the potential differential role of the local chaperone environment on the kinetically impaired ΔF508 fold.

### Example 8

### Effect of Co-Chaperone Aha1 on ΔF508 Folding in HEK293 Cells

The role of the co-chaperone regulator Aha1 in HEK293 cells stably expressing ΔF508 was examined. The most recently recognized member of the Hsp90 co-chaperone family is Aha1. Aha1 binds the middle domain of Hsp90 and is proposed to function as an ATPase activating protein regulating the ATP cycle of Hsp90 (Harst et al., 2005, Biochem J 387, 789-796; Mayer et al., 2002, Mol Cell 10, 1255-1256; Meyer, 2004, Embo J 23, 1402-1410; Meyer et al., 2003, Mol Cell 11, 647-658; Panaretou et al., 2002, Mol Cell 10, 1307-1318; Siligardi et al., 2004, J Biol Chem 279, 51989-51998).

dsRNA and transient transfection were used to control the level of protein expression of Aha1. The cDNA clone for Aha1 was amplified by PCR with a C-terminal myc-tag and cloned into pcDNA3.1+ and the sequence verified by sequencing. dsRNA solutions were prepared as in Example 5 but with 2 μM human Aha1 using 1 μM each of two dsRNAs (Dharmacon, Lafyette, Colo.) directed to human Aha1 sequences attggtccacggataagct (SEQ ID NO: 9; mRNA transcript SEQ ID NO: 12) and gtgagtaagcttgatggag (SEQ ID NO: 10; mRNA transcript SEQ ID NO: 18), and 6 μl of HiPerFect (Qiagen, Valencia, Calif.) per well of a 12 well dish. Control dsRNA (Dharmacon Cat. No. CONJB-000015) was added at equal concentration to Aha1 dsRNA. Over-expression of human Aha1 was performed by co-transfecting HEK293 with plasmids expressing CFTR ΔF508 and Aha1 by vaccinia virus infection as previously described (Wang et al., 2004, supra). For samples analyzed at the permissive temperature cells were shifted to 30° C. for 15 h prior to harvesting. SDS-PAGE and immunoblotting were as described in Example 3.

FIG. 6 is a series of bar graphs illustrating that ΔF508 export to the cell surface can be rescued by downregulation of functional Aha1. FIG. 6A is a set of bar graphs showing percent maximum levels for the steady-state pool of ER glycoform ΔF508 (B), cell surface glycoform ΔF508 (C), and Aha1 expression. Human Aha1 dsRNA (left panels) or human Aha1 cDNA (right panels) were used to reduce or overexpress, respectively, Aha1 in HEK293 cells expressing ΔF508 at 37° C. (upper panels) or 30° C. (lower panels). The insets are images of an SDS-PAGE immunoblot for the steady-state pool of ER glycoform (band B) and cell surface glycoform (band C). FIG. 6B is as described for 5A except that human Aha1 dsRNA was used to reduce Aha1 expression in CFBE41o-cells expressing ΔF508 at 37° C. (left panel) or 30° C. (right panel). The asterisks (*) indicate statistical significance (p≦0.05) using an unpaired, two-tailed t-test (triplicate samples). Representative results shown in triplicate from 4 independent experiments. For further methodology information, see Examples 8-9.

Results showed that dsRNA reduction of the endogenous level of Aha1 in HEK293 cells by 50-70% effected a marked 3- to 4-fold stabilization of ΔF508 band B (see e.g., FIG. 6A, upper left panel). An even more pronounced stabilization (4- to 5-fold) was observed at 30° C. (see e.g., FIG. 6A, lower left panel). Strikingly, at both 37° C. and 30° C., stabilization was associated with a corresponding increase in band C reflecting significant cell surface delivery (see e.g., FIG. 6A, left panels), a result not observed with the other co chaperones (see e.g., FIG. 3-5). Because the level of expression of Hsp90 co-chaperones can affect folding and export of ΔF508 at both the permissive (30° C.) and restrictive (37° C.) folding temperatures, the ΔF508 CFTR may be kinetically trapped in an on-pathway, metastable folded state(s) in response to the endogenous cytosolic pool of Hsp90 co-chaperones that normally facilitate folding of wild-type CFTR. The rescued band C was resistant to processing by endoglycosidase H (not shown), a hallmark of transport through the Golgi complex. In contrast to the effects of dsRNA, overexpression (4-fold) of Aha1 in HEK293 cells expressing ΔF508 significantly destabilized band B at both 37° C. (−60%) and 30° C. (>90%) with a corresponding loss of processing to band C (see e.g., FIG. 6A, right panels). Under these conditions, change in total pools of Hsc-Hsp70 or BIP was not detected, indicating that it is unlikely that a general ER stress response (Schroder and Kaufman, 2005, Annu Rev Biochem 74, 739-789) was induced by a modest reduction of Aha1 (results not shown).

By analogy to the dynamic role of Hsp90 in known folding pathways (Wegele et al., 2004, supra; Pratt and Toft, 2003, Exp Biol Med (Maywood) 228, 111-133; Prodromou and Pearl, 2003, supra), the above results suggest that regulation of the CFTR client interaction with Hsp90 through sequential interaction with co-chaperones may temporally coordinate steps in intradomain folding and/or coordinate inter-domain folding to avoid ERAD in the process of achieving the wild-type conformation. The need to coordinate intra- and interdomain folding is consistent with evidence that ΔF508 cannot achieve the proper interdomain interactions of NBD1 with TMD1 to produce a stable fold (Riordan, 2005, supra; Du et al., 2005, Nat Struct Mol Biol 12, 17-25). Moreover, the NBD2 domain, again temporally separated by synthesis of TMD2 (Riordan, 2005, supra), is misfolded in cells expressing ΔF508 CFTR and must dimerize with the NBD1 domain to activate one of the nucleotide binding pockets for channel function (Lewis, 2004, Embo J 23, 282-293). Stalled intermediates in the ΔF508 folding pathway are likely targets for recruitment of components such as CHIP and their regulatory factors HsBP1 and Bag-1/2 that bind the Hsc-Hsp70/40 complex and target of CFTR to ERAD (Alberti et al., 2004, Mol Biol Cell 15, 4003-4010,; Meacham et al., 2001, supra). It is now likely the relative abundance and perhaps the balance of specific regulators and co-chaperone components for both Hsc-Hsp70/40 and Hsp90 significantly influence the ability of wildtype and ΔF508 CFTR to fold for export from the ER. This conclusion is consistent with the general observation that ER stability and cell surface availability of ΔF508 is highly variable among different cell types.

### Example 9

### Effect of Co-Chaperone Aha1 on ΔF508 Folding in CFBE41o-Cells

Because HEK293 cells do not normally express ΔF508 and therefore may represent a special condition that is uniquely sensitive to the level of Aha1 activity (see Example 8), the effect of Aha1 dsRNA at 37° C. was examined in 1 lung cell line (CFBE41o-) that expresses endogenous levels of ΔF508.

Human bronchial cell line CFBE41o-derived from a CF patient homozygous for ΔF508 CFTR and the corrected HBE cell line was maintained in MEM supplemented with 10% FBS, Pen/Strep, 2 mM extra glutamine, and 2 μg/ml puromycin. dsRNA preparation and transfection, dsRNA solutions, and over expression of Aha1 were as described in Example 8. SDS-PAGE and immunoblotting were as described in Example 3.

Similar to the result observed in HEK293 cells, reduction of Aha1 resulted in stabilization of band B (−4-mold) compared to the scrambled control, with a corresponding 4- to 5-fold increase of band C either at 37° C. (see e.g., FIG. 6B, left panel) or 30° C. (see e.g., FIG. 6B, right panel), a level greater than that observed in the corrected CFBE41o-cell line (HBE) that expresses will-type CFTR (Bruscia et al., 2002, Gene Ther 9, 683-685) (see below). Pulse-chase analysis in CFBE41o-expressing ΔF508 revealed a 2-3 fold stabilization of band B in the ER preceding export to the cell surface (not shown). In contrast, no effect of Aha1 dsRNA was observed on stabilization of wild-type CFTR using pulse-chase analysis (not shown) or the steady-state cell surface levels of band C using the HBE cell line, suggesting that it is the ΔF508 mutant that requires an adjustment to the endogenous Aha1 pool to promote more efficient export.

The stabilization of band B and recovery of ΔF508 band C in response to altered levels of Aha1 suggests that reduced Aha1 activity may regulate Hsp90 chaperone dynamics to promote coupling of ΔF508 to the COPII ER export machinery.

### Example 10

### siRNA Design for Silencing Aha1

Candidate shRNA sequences for targeting the Aha1 gene were obtained from a search tool from Ambion (http://www.ambion.com/techlib/misc/siRNA_finder.html). The cDNA sequence of hAha1 (Ensembl Accession No. ENSG00000100591) was used to generate the exemplary listing. The sequences provide less than 50% GC content and avoid four or more Gs or Cs in a row. The complete listing is provided in Table 1.

Each shRNA was BLASTed against the human genome to identify possible off-target sites in other genes. Three dsRNAs chosen having a GC content around 40% and few off-target sites in other genes were selected: 99 (SEQ ID NO: 12), 179 (SEQ ID NO: 18) and 256 (SEQ ID NO: 24). Each of these three 19 base pair sequences has no more than 15 consecutive identities with any 5′ or 3′ untranslated regions, introns or exons of any other genes. The three sequences were cloned into the pSilencer vector (Ambion) to make stable Hela lines with reduced hAha1 expression. dsRNAs corresponding to the 99 and 179 sequences above were created (Dharmacon and Qiagen) for experiments provided herein.

### Example 11

### Aha 1 dsRNA Effect on Halide Conductance in CFBE41o-Cells Expressing ΔF508

While processing to the endo H resistant band C glycoform is a hallmark of transport from the ER to the cis/medial Golgi compartments (see e.g., Examples 8-9), it is possible that the rescued protein was trapped in late trans Golgi or endocytic compartments reflecting unanticipated contribution(s) of the Phe 508 deletion to abnormal sorting in post-ER pathways (see e.g., FIG. 1) (Gentzsch et al., 2004a; Sharma et al., 2004; Swiatecka-Urban et al., 2005). To test for this possibility, CFBE41o-cells expressing ΔF508 were treated with Aha1 dsRNA and surface halide conductance measured using an iodide efflux assay (Loo et al., 2005, supra).

As a positive control, the halide conductance of the corrected HBE cell line expressing wild-type CFTR was examined. For the Iodide efflux assay, wild type and ΔF508 CFBE41o-cells were seeded at a density of 5.0×10⁵ cells per 60 mm dish and grown under the conditions listed above for 5 days with a change of culture media every 2 days. dsRNA treatment was performed as indicated above with 20 μl of HiPerFect (Qiagen) per 60 mm dish. CFBE41o-cells were shifted to the permissive temperature of 30° C. for 15 h prior to iodide efflux analysis (Hughes et al., 2004). Cells were washed 5× with loading buffer (136 mM NaI; 3 mM KNO₃; 2 mM Ca(NO₃)₂; 20 mM Hepes and 11 mM glucose) and incubated for 1 h at room temperature with 2.5 ml of loading buffer. Cells were subsequently washed 15× with efflux buffer (136 mM NaNO₃; 3 mM KNO₃; 2 mM Ca(NO₃)₂; 20 mM Hepes and 11 mM glucose) and incubated with 2.5 ml of efflux buffer for 1 minute at room temperature and the media collected for analysis. This incubation was repeated for a total of 4 min. The cells were subsequently incubated with 2.5 ml of stimulation buffer (efflux buffer containing 10 μM forskolin (Sigma) and 50 μM genistein (Sigma)) for 1 min at room temperature and the media collected for analysis. This incubation was repeated for a total of 4 min. The cells were then incubated with 2.5 ml of efflux buffer for 1 min at room temperature and the media collected for analysis. This incubation was repeated for a total of 12 min. The samples were analyzed for iodide content using an iodide selective electrode (Analytical Sensors & Instruments) and a Beckman model 360 pH meter (VWR). The amount of iodide in the collected media was determined by extrapolating from a standard curve of known NaI concentrations. SDS-PAGE and immunoblotting were as described in Example 3.

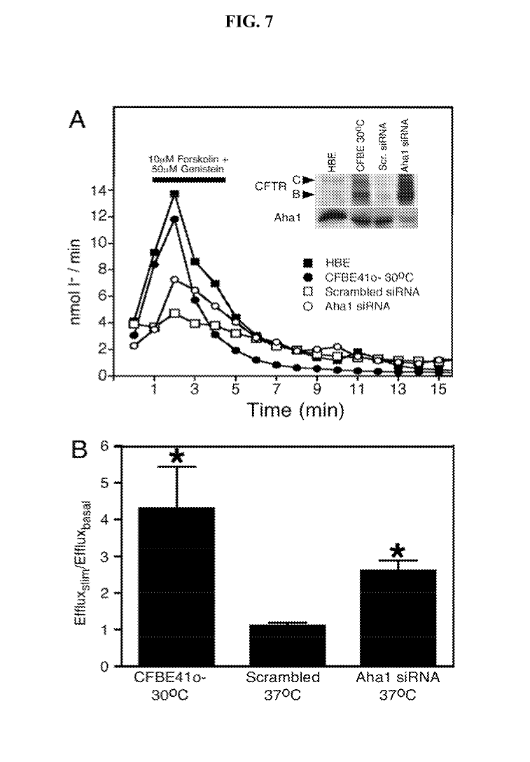

FIG. 7 is a line and scatter plot and a bar graph showing the effect of dsRNA Aha1 on iodide efflux by the CFBE41o-cell line. FIG. 7A is a line and scatter plot depicting iodide efflux over time. Iodide efflux was monitored in HBE cells expressing wild-type CFTR (closed boxes) or in ΔF508 expressing CFBE41o-cells that had been incubated at 37° C., or where indicated, at the permissive temperature of 30° C. (final 15 h) (closed circles), and transfected with Aha1 (open circles) or scrambled (control) (open boxes) dsRNA. CFTR channels were activated by addition of 10 μM forskolin and 50 μM genistein over a 4 min period starting at 1 min and subsequently washed out with efflux buffer. The effect of temperature-shift and dsRNA on CFTR maturation (band B to band C glycoforms) and Aha1 stability is shown in the inset. FIG. 7B is a bar graph depicting the ratio of halide conductance prior to addition of forskolin/genistein (0 min) and at 2 min, the peak period of halide flux. The asterisks (*) indicate statistical significance (p≦0.05) using the unpaired, two-tailed t-test (triplicate samples) between the temperature-corrected (lane a) and dsRNA-treated (lane c) CFBE41o-cells compared to the scrambled dsRNA-treated control (lane b). There was no statistically significant difference between halide conductance for temperature-corrected (lane a) and dsRNA corrected CFBE41O-cells (lane c) (p=0.2). Experiments were repeated independently at least three times with representative results shown. For further methodology information, see Example 10.

Results showed that treatment of the CFBE41o-cell line with Aha1 dsRNA resulted in ˜70-80% knock-down of endogenous Aha1, leading to stabilization of ΔF508 band B and C at levels ˜1.5-fold the 30° C. to temperature-corrected control and a 4-fold stabilization of band B over the scrambled dsRNA treated cells (see e.g., FIG. 7A, insert). Whereas HBE cells showed strong halide conductance, no conductance was detected in control CFBE41o-cells that were treated with scrambled dsRNA (see e.g., FIG. 7A). Shift of CFBE41o- to 30° C. resulted in recovery of 80-90% of the conductance observed in HBE cells (see e.g., FIG. 7A). Strikingly, CFBE41o-cells treated with dsRNA, but not scrambled, showed 50-80% recovery of halide conductance compared to that observed in temperature-corrected cells (see e.g., FIG. 7B). These results demonstrate that Aha 1 dsRNA restores halide conductance to CFBE41o-cells expressing ΔF508, thereby achieving functional rescue of CFTR.

FIG. 8 is a series of cartoons depicting Hsp90 chaperone/co-chaperone interactions directing CFTR folding. FIG. 8A is a cartoon highlighting components involved in wild-type and ΔF508 CFTR folding. They consist of lumenal chaperones (1), and a two-state cytosolic system that includes the core components Hsc-Hsp70/40 (2) and Hsp90 (3) as well as number of Hsc-Hsp70 (2) and Hsp90 (3) co-chaperone regulator. Additional chaperones such as TCP1 (Spiess et al., 2004, supra) and Hsp105/S100 (3a) may also contribute to folding. One or more of these protein interactions are kinetically disrupted by the Phe 508 deletion leading disruption of the Hsp90 ATPase cycle and CF pathophysiology. FIG. 8B is an illustration of the potential role of Hsp90 and the co-chaperones in folding and rescue of ΔF508 CFTR. The ATP/ADP cycle regulating folding for export through ERAF or targeting for ERAD can be dynamically controlled by co-chaperone regulators (X and Y) to adjust the kinetics of the chaperone cycle to the kinetics and energetics of the folding pathway. For example, down-regulation of Aha1 ATPase activity by dsRNA (X) would favor stabilization of ΔF508 for export by reducing Hsp90 ATPase activity, whereas down-regulation of p23 (Y) would favor destabilization leading to ERAD. FIG. 8C is a plot illustrating the relationship between the (co)chaperone concentration in the cytosol (X axis), a hypothetical ‘folding stability score’ defined by global protein energetics (Sekijima et al., 2005, Cell 121, 73-85) (Z axis), and ‘export efficiency’ reflecting the level of transport to the cell surface (Y axis). Whereas the more energetically stable wild-type CFTR (dashed curve) responds to the folding activity of the CFTR chaperone response to the normal concentration of Aha1 (box having grid, ‘normal chaperone’), the reduced folding energetics of ΔF508 (left solid curve) is unstable in this folding environment and fails to be exported. A change in the set-point of the Hsp90 ATP/ADP cycle afforded by downregulation of Aha1 (grey box, ‘rescue chaperone’) provides a more productive solvent by adjusting chaperone folding capacity (grid box) to folding of ΔF508 (left curve), while maintaining functionality of the wild-type CFTR fold (right solid curve, grid box). Geldanamycin (GA), a Hsp90 inhibitor, blocks both wild-type and ΔF508 CFTR folding and export by directly binding to Hsp90 and arresting the folding cycle (lower left corner) (Loo et al., 1998, supra).

In summary, the results above suggest that the intrinsic folding defect in mutant CFTR is kinetically linked to the activity of the Aha1-sensitive Hsp90 ATPase cycle. The working model developed from results herein emphasizes an environment-sensitive uncoupling from normal cellular folding pathways. In this view, the intrinsic rate of the Hsp90 ATP/ADP cycle controlling Hsp90-client complex interactions is coordinated with the energetics of folding of wild-type CFTR through co-chaperone activity. Whereas the folding energetics driving export of wildtype CFTR is optimized relative to the normal cellular chaperone pool (FIG. 8), a change in the activities of Aha1, and potentially other cochaperones, can alter these (FIG. 8) and the capacity of the chaperone folding/export pathway. In the case of Aha1, reduction of Hsp90 ATPase activity may allow additional time for the kinetically challenged ΔF508mutant (FIG. 8) to engage a ‘rescue’ chaperone pool (FIG. 8) to favor stability and folding for export. Because partial reduction of the Aha1 pool did not significantly impair the more energetically stable wild-type fold (FIG. 8), these results emphasize that the folding energetics of the Phe 508 deletion may lie outside the normal chaperoned folding boundaries. The ability of a unique local population of chaperones to modulate folding is consistent with recent observations that folding chaperones are now found to regulate specific cellular protein folding pathways (Albanese et al., 2006, Cell 124, 75-88), rather than simply function as inhibitors of protein aggregation (Wickner et al., 1999, Science 286, 1888-1893).

From an evolutionary perspective, genetic modifiers (Qu and Thomas, 1996, supra) are now likely to include folding chaperones that provide a favorable genetic or epigenetic (Cowen and Lindquist, 2005, Science 309, 2185; Queitsch et al., 2002, Nature 417, 618) environment for reduced function of the mutant, yet survival value when challenged with agonists such as cholera toxin where reduced chloride channel function would decrease the possibility of dehydration and death when compared to the wild-type population (Gabriel et al., 1994, Science 266, 107; Thiagarajah and Verkman, 2005, Trends Pharmacol Sci 26, 172). Thus, the activity of chaperone pools may define the difference between a tolerated polymorphism and a deleterious mutation in CF and other protein misfolding diseases.

### Example 12

### Hsp90 Binding to CFTR is Responsive to Aha1 Activity

To determine the effect of Aha1 knock-down on the interaction of ΔF508 with Hsp90, we analyzed the recovery of Hsp90 bound to CFTR following treatment of cells with Aha1 dsRNA. Cells expressing ΔF508 at 37° C. were incubated in presence of scrambled or Aha1 dsRNA. Cells were harvested, CFTR immunoprecipitated, and the amount of Hsp90 associated with ΔF508 quantified by immunoblotting. For these experiments, we analyzed the ratio of Hsp90 to CFTR recovered in the immunoprecipitate to determine the relative amount of Hsp90 bound to CFTR under control or knock-down conditions.

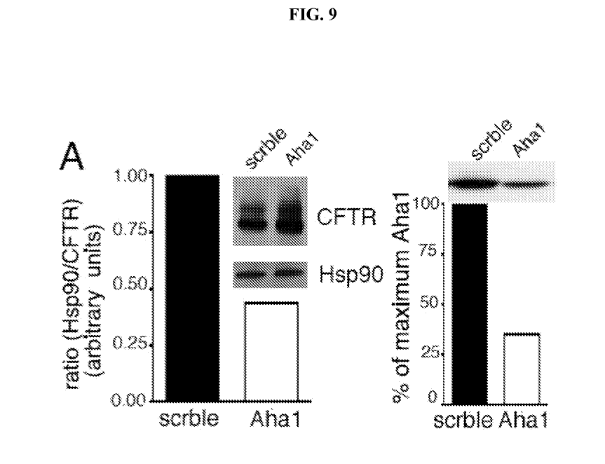

FIG. 9 illustrates effects of dsRNA Aha1 on Hsp90. HEK293 cells expressing ΔF508 at 37° C. were incubated in absence or presence Aha1 dsRNA. Cells were harvested, CFTR immunoprecipitated and the amount of Hsp90 recovered with ΔF508 was quantified by immunoblotting. Left panel: Ratio of Hsp90 to CFTR recovered in the immunoprecipitate. Right panel: fraction of Aha1 remaining in cells following Aha1 dsRNA treatment compared to scrambled control.

Under conditions in which we observed an ˜60% knock-down of Aha1 (FIG. 9, right panel), we observed a 50-60% decrease of bound Hsp90 at reduced levels of Aha1 (FIG. 9, left panel). In contrast, under these conditions we detected no change in the cellular levels of calnexin, BiP, Hsp40, Hsc-Hsp70, Hsp90, HOP FKBP8/38 and p23 compared to the scrambled control, indicating that a reduction in Aha1 can alter the steady-state pool of ΔF508 associated with Hsp90 in the ER. This result is consistent with the observation that Hsp90 and Hsp90 co-chaperone recovery in the CFTR wild-type interactome is reduced relative to the ΔF508 interactome despite comparable levels of band B. The results demonstrate that lowering the level of the Aha1 co-chaperone regulator can modify the kinetic interactions of ΔF508 with Hsp90 to facilitate more efficient progression through the folding pathway, thereby favoring export.

Other Aspects

The detailed description set-forth above is provided to aid those skilled in the art in practicing the present invention. However, the invention described and claimed herein is not to be limited in scope by the specific aspects herein disclosed because these aspects are intended as illustration of several aspects of the invention. Any equivalent aspects are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

Citation of a reference herein shall not be construed as an admission that such is prior art to the present invention. Specifically intended to be within the scope of the present invention, and incorporated herein by reference in its entirety for all purposes, is the following publication: Wang, X. et al., Hsp90 cochaperone Aha1 downregulation rescues misfolding of CFTR in cystic fibrosis, Cell (2006 Nov. 17) 127(4):673-5.

What is claimed is:

1. A dsRNA for inhibiting functional Aha protein expression in a cell, said dsRNA comprising a sense strand and an antisense strand,
wherein said antisense strand comprises a region of complementarity having a sequence substantially complementary to an Aha target sequence, wherein said target sequence is less than 30 nucleotides in length,
wherein said sense strand is substantially complimentary to said antisense strand, and
wherein said dsRNA, upon contact with a cell expressing functional Aha protein, inhibits functional Aha protein expression by at least 20%.

2. A dsRNA according to claim 1, wherein said Aha target sequence comprises a sequence selected from the group consisting of SEQ ID NOs: 12-56.

3. A dsRNA according to claim 1, wherein said dsRNA comprises a sense strand having a sequence selected from the group consisting of SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, and SEQ ID NO: 145; and
an antisense strand complementary to the sense strand having a sequence selected from the group consisting of SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, and SEQ ID NO: 146.

4. A dsRNA according to claim 1, wherein said dsRNA comprises a sense strand having a sequence of SEQ ID NO: 57 and an antisense strand complementary to the sense strand having a sequence of SEQ ID NO: 58.

5. A dsRNA according to claim 1, wherein said dsRNA comprises a sense strand having a sequence of SEQ ID NO: 69 and an antisense strand complementary to the sense strand having a sequence of SEQ ID NO: 70.

6. A dsRNA according to claim 1, wherein said dsRNA comprises a sense strand having a sequence of SEQ ID NO: 81 and an antisense strand complementary to the sense strand having a sequence of SEQ ID NO: 82.

7. A vector for expressing a shRNA for inhibiting functional Aha1 expression in a cell, said vector comprising a sense strand, a hairpin linker, and an antisense strand,
wherein said sense strand comprising a region of complementarity having a sequence substantially complementary to an Aha target sequence, wherein said target sequence is less than 30 nucleotides in length,
wherein said antisense strand is substantially complimentary to said sense strand, and
wherein said dsRNA, upon contact with a cell expressing functional Aha protein, inhibits functional Aha protein expression by at least 20%.

8. A vector according to claim 7, wherein said Aha target sequence comprises a sequence selected from the group consisting of SEQ ID NOs: 12-56.

9. A vector according to claim 7, wherein said vector comprises a sense strand having a sequence selected from the group consisting of SEQ ID NO: 147, SEQ ID NO: 149, and SEQ ID NO: 151; and
an antisense strand having a sequence selected from the group consisting of SEQ ID NO: 148, SEQ ID NO: 150, and SEQ ID NO: 152.

10. A shRNA for inhibiting functional Aha1 protein expression in a cell, said shRNA comprising a region of complementarity having a sequence substantially complementary to an Aha target sequence, and wherein said target sequence is less than 30 nucleotides in length, and
wherein said shRNA, upon contact with a cell expressing functional Aha protein, inhibits functional Aha protein expression by at least 20%.

11. A shRNA according to claim 10, wherein said Aha target sequence comprises a sequence selected from the group consisting of SEQ ID NOs: 12-56.

12. A shRNA according to claim 10, wherein said shRNA comprises a sequence selected from the group consisting of SEQ ID NO: 153, SEQ ID NO: 154, and SEQ ID NO: 155;

13. A cell or cell population comprising a dsRNA according to claim 1.

14. A cell or cell population comprising a vector according to claim 7.

15. A cell or cell population comprising a shRNA according to claim 10.

16. An isolated antibody that specifically binds functional Aha1, the Hsp90 ATPase binding site for functional Aha1, and/or the functional Aha1-Hsp90 ATPase complex.

17. An agent that decreases intracellular levels of functional Aha1 protein, said agent selected from the group consisting of a small molecule, an antibody, an antisense nucleic acid, an aptamer, a dsRNA, a ribozyme, and any combination thereof.

18. A method of treating a disease associated with misfolding of a protein, the method comprising administering to a subject in need thereof a therapeutically effective amount of at least one agent that decreases intracellular levels of functional Aha1 protein, wherein said agent is selected from the group consisting of a small molecule, an antibody, an antisense nucleic acid, an aptamer, an siRNA, a ribozyme, and combinations thereof.

19. A method according to claim 18, wherein the disease is selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease.

20. A method according to claim 18, wherein the disease is CF.

21. A method according to claim 18, wherein the misfolded protein is a misfolded CFTR.

22. A method according to claim 18, wherein the misfolded protein is a ΔF508 protein.

23. A method of treating a disease associated with misfolding of a protein, the method comprising administering to a subject in need thereof a therapeutically effective amount of at least one dsRNA inhibitor of functional Aha1 expression, said dsRNA comprising a sense strand and an antisense strand,
wherein said antisense strand comprises a region of complementarity having a sequence substantially complementary to an Aha target sequence, wherein said target sequence is less than 30 nucleotides in length,
wherein said sense strand is substantially complimentary to said antisense strand, and
wherein said dsRNA, upon contact with a cell expressing functional Aha protein, inhibits functional Aha protein expression by at least 20%.

24. A method according to claim 23, wherein the disease is selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease.

25. A method according to claim 23, wherein the disease is CF.

26. A method according to claim 23, wherein the misfolded protein is a misfolded CFTR.

27. A method according to claim 23, wherein the misfolded protein is a ΔF508 protein.

28. A method of treating a disease associated with misfolding of a protein, the method comprising administering to a subject in need thereof a therapeutically effective amount of at least one dsRNA inhibitor of functional Aha1 expression, wherein the dsRNA inhibitor comprises a sequence selected on the basis of
a) the dsRNA comprising a sense strand sequence of about 19 nucleotides to about 25 nucleotides and an antisense strand sequence of about 19 nucleotides to about 25 nucleotides; and
b) the sense strand sequence or antisense strand sequence comprises no more than 15 contiguous nucleotides identical to a contiguous sequence comprised by a 5′ untranslated region, a 3′ untranslated region, an intron or an exon of any gene or mRNA other than functional Aha1.

29. A method according to claim 28, wherein the disease is selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease.

30. A method according to claim 28, wherein the disease is CF.

31. A method according to claim 28, wherein the misfolded protein is a misfolded CFTR.

32. A method according to claim 28, wherein the misfolded protein is a ΔF508 protein.

33. A method of screening an agent for treating a disease associated with misfolding of a protein, the method comprising:
providing a cell or cell population expressing functional Aha1;
administering a candidate agent to the cell or cell population;
quantifying functional Aha1 activity in the cell or cell population; and
determining whether the candidate agent decreases functional Aha1 activity in the cell or cell population, whereby a decrease in functional Aha1 activity is indicative of reducing misfolding of the protein.

34. A method according to claim 33, wherein the candidate agent is an dsRNA which inhibits functional Aha1 expression.

35. A method according to claim 34, wherein the dsRNA comprises a) a sequence of from about 19 nucleotides to about 25 nucleotides, and b) the sequence comprises no more than 15 contiguous nucleotides identical to a contiguous sequence comprised by a 5′ untranslated region, a 3′ untranslated region, an intron or an exon of any gene or mRNA other than an Aha gene or mRNA.

36. A method according to claim 35, wherein the Aha gene or mRNA is a human Aha gene or mRNA.

37. A method according to claim 33, wherein the disease is selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease.

38. A method according to claim 33, wherein the disease is CF.

39. A method according to claim 33, wherein the misfolded protein is selected from the group consisting of a misfolded CFTR, a misfolded fibrillin, a misfolded alpha galactosidase, a misfolded beta glucocerebrosidase, a misfolded rhodopsin, aggregated an amyloid beta and tau, an aggregated amylin, an aggregated alpha synuclein and an aggregated prion.

40. A method according to claim 33, wherein the misfolded protein is a misfolded CFTR.

41. A method according to claim 33, wherein the misfolded protein is a ΔF508 protein.

42. A method of screening for an agent for treating a disease associated with misfolding of a protein, the method comprising:
providing a cell or cell population which expresses functional Aha1;
administering a candidate agent to the cell or cell population;
quantifying Hsp90/ADP complex, Hsp90/ATP complex or a combination thereof in the cell or cell population; and
determining whether the candidate agent decreases the quantity of Hsp90/ADP complex, Hsp90/ATP complex or the combination thereof in the cell or cell population, whereby a decrease in quantity of Hsp90/ADP complex or Hsp90/ATP complex is indicative of decreasing misfolding of the protein.

43. A method according to claim 42, wherein the candidate agent is an dsRNA which inhibits functional Aha1 expression.

44. A method according to claim 43, wherein the dsRNA comprises a) a sequence of from about 19 nucleotides to about 25 nucleotides, and b) the sequence comprises no more than 15 contiguous nucleotides identical to a contiguous sequence comprised by a 5′ untranslated region, a 3′ untranslated region, an intron or an exon of any gene or mRNA other than an Aha gene or mRNA.

45. A method according to claim 44, wherein the Aha gene or mRNA is a human Aha gene or mRNA.

46. A method according to claim 42, wherein the disease is selected from the group consisting of cystic fibrosis (CF), Marfan syndrome, Fabry disease, Gaucher's disease, retinitis pigmentosa 3, Alzheimer's disease, Type II diabetes, Parkinson's disease and Creutzfeldt-Jakob disease.

47. A method according to claim 42, wherein the disease is CF.

48. A method according to claim 42, wherein the misfolded protein is selected from the group consisting of a misfolded CFTR, a misfolded fibrillin, a misfolded alpha galactosidase, a misfolded beta glucocerebrosidase, a misfolded rhodopsin, aggregated an amyloid beta and tau, an aggregated amylin, an aggregated alpha synuclein and an aggregated prion.

49. A method according to claim 42, wherein the misfolded protein is a misfolded CFTR.

50. A method according to claim 42, wherein the misfolded protein is a ΔF508 protein.


SEQUENCE LISTING (152 sequences)

SEQ ID NO: 1 - DNA, 6132 bp, Homo sapiens
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatataacaatgctgtattttaaaagaatgattatgaattacatttgtataaaataatttttatatttgaaatattgactttttatggcactagtatttctatgaaatattatgttaaaactgggacaggggagaacctagggtgatattaaccaggggccatgaatcaccttttggtctggagggaagccttggggctgatgcagttgttgcccacagctgtatgattcccagccagcacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgtgaaa

SEQ ID NO: 2 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 3 - protein, 1479 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 4 - protein, 338 aa, Homo sapiens
MAKWGEGDPRWIVEERADATNVNNWHWTERDASNWSTDKLKTLFLAVQVQNEEGKCEVTEVSKLDGEASINNRKGKLIFFYEWSVKLNWTGTSKSGVQYKGHVEIPNLSDENSVDEVEISVSLAKDEPDTNLVALMKEEGVKLLREAMGIYISTLKTEFTQGMILPTMNGESVDPVGQPALKTEERKAKPAPSKTQARPVGVKIPTCKITLKETFLTSPEELYRVFTTQELVQAFTHAPATLEADRGGKFHMVDGNVSGEFTDLVPEKHIVMKWRFKSWPEGHFATITLTFIDKNGETELCMEGRGIPAPEEERTRQGWQRYYFEGIKQTFGYGARLF

SEQ ID NO: 5 - protein, 338 aa, Bos taurus
MAKWGEGDPRWIVEERADATNVNNWHWTERDASNWSTDKLKTLFLAVRVQNEEGKCEVTEVSKLDGEASINNRKGKLIFFYEWSVKLNWTGTSKSGVQYKGHVEIPNLSDENSVDEVEISVSLAKDEPDTNLVALMKEEGVKLLREAMGIYISTLKTEFTQGMILPTMNGESVDPAGPPALKTEERKAKSAPSKTQARPVGVKIPTCKITLRESFLTSPEELYRVFTTQELVQAFTHAPAMLEADKGGKFHLVDGNVSGEFTDLVPEKYIAMKWRFKSWPEGHFAIITLTFIDKNGETELCMEGRGIPAPEEERTRQGWQRYYFEGIKQTFGYGARLF

SEQ ID NO: 6 - protein, 338 aa, Mus musculus
MAKWGEGDPRWIVEERADATNVNNWHWTERDASNWSTEKLKTLFLAVRVENEEGKCEVTEVNKLDGEASINNRKGKLIFFYEWTIKLNWTGTSKSGVQYKGHVEIPNLSDENSVDEVEISVSLAKDEPDTNLVALMKEDGVKLLREAVGIYISTLKTEFTQGMILPTVNGESVDPVGQPALKTETCKAKSAPSKSQAKPVGVKIPTCKITLKETFLTSPEELYRVFTTQELVQAFTHAPAALEADRGGKFHMVDGNVTGEFTDLVPEKHIAMKWRFKSWPEGHFATITLTFIDKNGETELCMEGRGIPAPEEERTRQGWQRYYFEGIKQTFGYGARLF

SEQ ID NO: 7 - protein, 336 aa, Xenopus laevis
MAKWGEGDPRWIVEMRADATNVNNWHWTERDATSWSLAKIKELMMGIRVESEEGTCDITEVSKLEGEASINNRKGKLIFFYEWDIKLNWTGVSKSGVKYKGYVEIPNLSDENDPSEVEIRVSMAKDEPETNLIGVMRKQGSKQIREAVAQYISMLKSEFTQGMILPTANGVSHNISEIKQKTETNMPQTGKTQTCQNAGVKIPTCKVTIKDTFLTSPEELYRVLTRQELVQGFTHAPASLTADKGGKFQLLGGNVSGEFKELEPEKHIVMSWRFKSWPQGHHASITLTFTDKGGETELWMEARGVPQGEEERTKEGWKRYYFDGIKQTFGYGALLL

SEQ ID NO: 8 - protein, 316 aa, Danio rerio
MLIIGTERDVTSWSQDAINGLLLGIRVEGEEGTCEITDVSNIDGEASINNRKGKLIYFYEWVVKASWTGTNKIGIKYKGIVEIPNLSDENDMDDLDISVTLCKDQPNTPLTDLMRREGVKKIRMALGNYVKHLKTEFAQGMILPTENALFQQNQEAKAKVKLDKTQIGSPSTANAPSTGVKIATVSFSLKDTFLTSPEELYRIFITQEMVQAFTHLAAFVDGRCGGKFRLLEGNVHGQFAELIPDKKIAMRWRFASWPAGHAATVILNFVNQGSETELILEAKGVPSNEEERMKEGWQRYYFNAIKQTFGFGALLY

SEQ ID NO: 9 - DNA, 19 bp, Homo sapiens
attggtccacggataagct

SEQ ID NO: 10 - DNA, 19 bp, Homo sapiens
gcgagtaagcttgatggag

SEQ ID NO: 11 - DNA, 1017 bp, Homo sapiens
atggccaagtggggtgagggagacccacgctggatcgtggaggagcgggcggacgccaccaacgtcaacaactggcactggacggagagagatgcttcaaattggtccacggataagctgaaaacactgttcctggcagtgcaggttcaaaatgaagaaggcaagtgtgaggtgacggaagtgagtaagcttgatggagaggcatccattaacaatcgcaaagggaaacttatcttcttttatgaatggagcgtcaaactaaactggacaggtacttctaagtcaggagtacaatacaaaggacatgtggagatccccaatttgtctgatgaaaacagcgtggatgaagtggagattagtgtgagccttgccaaagatgagcctgacacaaatctcgtggccttaatgaaggaagaaggggtgaaacttctaagagaagcaatgggaatttacatcagcaccctcaaaacagagttcacccagggcatgatcttacctacaatgaatggagagtcagtagacccagtggggcagccagcactgaaaactgaggagcgcaaggctaagcctgctccttcaaaaacccaggccagacctgttggagtcaaaatccccacttgtaagatcactcttaaggaaaccttcctgacgtcaccagaggagctctatagagtgtttaccacccaagagctggtgcaggcctttacccatgctcctgcaacattagaagcagacagaggtggaaagttccacatggtagatggcaacgtctctggggaatttactgatctggtccctgagaaacatattgtgatgaagtggaggtttaaatcttggccagagggacactttgccaccatcaccttgaccttcatcgacaagaacggagagactgagctgtgcatggaaggtcgaggcatccctgctcctgaggaagagcggacgcgacagggctggcagcggtactactttgagggcattaaacagacctttggctatggcgcacgcttattttag

SEQ ID NO: 12 - DNA, 21 bp, Homo sapiens
aaattggtccacggataagct

SEQ ID NO: 13 - DNA, 21 bp, Homo sapiens
aagctgaaaacactgttcctg

SEQ ID NO: 14 - DNA, 21 bp, Homo sapiens
aaaacactgttcctggcagtg

SEQ ID NO: 15 - DNA, 21 bp, Homo sapiens
aaaatgaagaaggcaagtgtg

SEQ ID NO: 16 - DNA, 21 bp, Homo sapiens
aatgaagaaggcaagtgtgag

SEQ ID NO: 17 - DNA, 21 bp, Homo sapiens
aagaaggcaagtgtgaggtga

SEQ ID NO: 18 - DNA, 21 bp, Homo sapiens
aagtgagtaagcttgatggag

SEQ ID NO: 19 - DNA, 21 bp, Homo sapiens
aacaatcgcaaagggaaactt

SEQ ID NO: 20 - DNA, 21 bp, Homo sapiens
aatcgcaaagggaaacttatc

SEQ ID NO: 21 - DNA, 21 bp, Homo sapiens
aaagggaaacttatcttcttt

SEQ ID NO: 22 - DNA, 21 bp, Homo sapiens
aaacttatcttcttttatgaa

SEQ ID NO: 23 - DNA, 21 bp, Homo sapiens
aatggagcgtcaaactaaact

SEQ ID NO: 24 - DNA, 21 bp, Homo sapiens
aaactaaactggacaggtact

SEQ ID NO: 25 - DNA, 21 bp, Homo sapiens
aaactggacaggtacttctaa

SEQ ID NO: 26 - DNA, 21 bp, Homo sapiens
aagtcaggagtacaatacaaa

SEQ ID NO: 27 - DNA, 21 bp, Homo sapiens
aatacaaaggacatgtggaga

SEQ ID NO: 28 - DNA, 21 bp, Homo sapiens
aatttgtctgatgaaaacagc

SEQ ID NO: 29 - DNA, 21 bp, Homo sapiens
aaaacagcgtggatgaagtgg

SEQ ID NO: 30 - DNA, 21 bp, Homo sapiens
aagtggagattagtgtgagcc

SEQ ID NO: 31 - DNA, 21 bp, Homo sapiens
aaagatgagcctgacacaaat

SEQ ID NO: 32 - DNA, 21 bp, Homo sapiens
aaatctcgtggccttaatgaa

SEQ ID NO: 33 - DNA, 21 bp, Homo sapiens
aatgaaggaagaaggggtgaa

SEQ ID NO: 34 - DNA, 21 bp, Homo sapiens
aaggaagaaggggtgaaactt

SEQ ID NO: 35 - DNA, 21 bp, Homo sapiens
aagaaggggtgaaacttctaa

SEQ ID NO: 36 - DNA, 21 bp, Homo sapiens
aaggggtgaaacttctaagag

SEQ ID NO: 37 - DNA, 21 bp, Homo sapiens
aaacttctaagagaagcaatg

SEQ ID NO: 38 - DNA, 21 bp, Homo sapiens
aagagaagcaatgggaattta

SEQ ID NO: 39 - DNA, 21 bp, Homo sapiens
aagcaatgggaatttacatca

SEQ ID NO: 40 - DNA, 21 bp, Homo sapiens
aatgggaatttacatcagcac

SEQ ID NO: 41 - DNA, 21 bp, Homo sapiens
aatttacatcagcaccctcaa

SEQ ID NO: 42 - DNA, 21 bp, Homo sapiens
aatgaatggagagtcagtaga

SEQ ID NO: 43 - DNA, 21 bp, Homo sapiens
aatggagagtcagtagaccca

SEQ ID NO: 44 - DNA, 21 bp, Homo sapiens
aagcctgctccttcaaaaacc

SEQ ID NO: 45 - DNA, 21 bp, Homo sapiens
aaaatccccacttgtaagatc

SEQ ID NO: 46 - DNA, 21 bp, Homo sapiens
aatccccacttgtaagatcac

SEQ ID NO: 47 - DNA, 21 bp, Homo sapiens
aagatcactcttaaggaaacc

SEQ ID NO: 48 - DNA, 21 bp, Homo sapiens
aaggaaaccttcctgacgtca

SEQ ID NO: 49 - DNA, 21 bp, Homo sapiens
aacattagaagcagacagagg

SEQ ID NO: 50 - DNA, 21 bp, Homo sapiens
aagcagacagaggtggaaagt

SEQ ID NO: 51 - DNA, 21 bp, Homo sapiens
aaagttccacatggtagatgg

SEQ ID NO: 52 - DNA, 21 bp, Homo sapiens
aacgtctctggggaatttact

SEQ ID NO: 53 - DNA, 21 bp, Homo sapiens
aatttactgatctggtccctg

SEQ ID NO: 54 - DNA, 21 bp, Homo sapiens
aaacatattgtgatgaagtgg

SEQ ID NO: 55 - DNA, 21 bp, Homo sapiens
aagtggaggtttaaatcttgg

SEQ ID NO: 56 - DNA, 21 bp, Homo sapiens
aaacagacctttggctatggc

SEQ ID NO: 57 - RNA, 19 nt, Homo sapiens
auugguccacggauaagcu

SEQ ID NO: 58 - RNA, 19 nt, Homo sapiens
agcuuauccguggaccaau

SEQ ID NO: 59 - RNA, 19 nt, Homo sapiens
gcugaaaacacuguuccug

SEQ ID NO: 60 - RNA, 19 nt, Homo sapiens
caggaacaguguuuucagc

SEQ ID NO: 61 - RNA, 19 nt, Homo sapiens
aacacuguuccuggcagug

SEQ ID NO: 62 - RNA, 19 nt, Homo sapiens
cacugccaggaacaguguu

SEQ ID NO: 63 - RNA, 19 nt, Homo sapiens
aaugaagaaggcaagugug

SEQ ID NO: 64 - RNA, 19 nt, Homo sapiens
cacacuugccuucuucauu

SEQ ID NO: 65 - RNA, 19 nt, Homo sapiens
ugaagaaggcaagugugag

SEQ ID NO: 66 - RNA, 19 nt, Homo sapiens
cucacacuugccuucuuca

SEQ ID NO: 67 - RNA, 19 nt, Homo sapiens
gaaggcaagugugagguga

SEQ ID NO: 68 - RNA, 19 nt, Homo sapiens
ucaccucacacuugccuuc

SEQ ID NO: 69 - RNA, 19 nt, Homo sapiens
gugaguaagcuugauggag

SEQ ID NO: 70 - RNA, 19 nt, Homo sapiens
cuccaucaagcuuacucac

SEQ ID NO: 71 - RNA, 19 nt, Homo sapiens
caaucgcaaagggaaacuu

SEQ ID NO: 72 - RNA, 19 nt, Homo sapiens
aaguuucccuuugcgauug

SEQ ID NO: 73 - RNA, 19 nt, Homo sapiens
ucgcaaagggaaacuuauc

SEQ ID NO: 74 - RNA, 19 nt, Homo sapiens
gauaaguuucccuuugcga

SEQ ID NO: 75 - RNA, 19 nt, Homo sapiens
agggaaacuuaucuucuuu

SEQ ID NO: 76 - RNA, 19 nt, Homo sapiens
aaagaagauaaguuucccu

SEQ ID NO: 77 - RNA, 19 nt, Homo sapiens
acuuaucuucuuuuaugaa

SEQ ID NO: 78 - RNA, 19 nt, Homo sapiens
uucauaaaagaagauaagu

SEQ ID NO: 79 - RNA, 19 nt, Homo sapiens
uggagcgucaaacuaaacu

SEQ ID NO: 80 - RNA, 19 nt, Homo sapiens
aguuuaguuugacgcucca

SEQ ID NO: 81 - RNA, 19 nt, Homo sapiens
acuaaacuggacagguacu

SEQ ID NO: 82 - RNA, 19 nt, Homo sapiens
aguaccuguccaguuuagu

SEQ ID NO: 83 - RNA, 19 nt, Homo sapiens
acuggacagguacuucuaa

SEQ ID NO: 84 - RNA, 19 nt, Homo sapiens
uuagaaguaccuguccagu

SEQ ID NO: 85 - RNA, 19 nt, Homo sapiens
gucaggaguacaauacaaa

SEQ ID NO: 86 - RNA, 19 nt, Homo sapiens
uuuguauuguacuccugac

SEQ ID NO: 87 - RNA, 19 nt, Homo sapiens
uacaaaggacauguggaga

SEQ ID NO: 88 - RNA, 19 nt, Homo sapiens
ucuccacauguccuuugua

SEQ ID NO: 89 - RNA, 19 nt, Homo sapiens
uuugucugaugaaaacagc

SEQ ID NO: 90 - RNA, 19 nt, Homo sapiens
gcuguuuucaucagacaaa

SEQ ID NO: 91 - RNA, 19 nt, Homo sapiens
aacagcguggaugaagugg

SEQ ID NO: 92 - RNA, 19 nt, Homo sapiens
ccacuucauccacgcuguu

SEQ ID NO: 93 - RNA, 19 nt, Homo sapiens
guggagauuagugugagcc

SEQ ID NO: 94 - RNA, 19 nt, Homo sapiens
ggcucacacuaaucuccac

SEQ ID NO: 95 - RNA, 19 nt, Homo sapiens
agaugagccugacacaaau

SEQ ID NO: 96 - RNA, 19 nt, Homo sapiens
auuugugucaggcucaucu

SEQ ID NO: 97 - RNA, 19 nt, Homo sapiens
aucucguggccuuaaugaa

SEQ ID NO: 98 - RNA, 19 nt, Homo sapiens
uucauuaaggccacgagau

SEQ ID NO: 99 - RNA, 19 nt, Homo sapiens
ugaaggaagaaggggugaa

SEQ ID NO: 100 - RNA, 19 nt, Homo sapiens
uucaccccuucuuccuuca

SEQ ID NO: 101 - RNA, 19 nt, Homo sapiens
ggaagaaggggugaaacuu

SEQ ID NO: 102 - RNA, 19 nt, Homo sapiens
aaguuucaccccuucuucc

SEQ ID NO: 103 - RNA, 19 nt, Homo sapiens
gaaggggugaaacuucuaa

SEQ ID NO: 104 - RNA, 19 nt, Homo sapiens
uuagaaguuucaccccuuc

SEQ ID NO: 105 - RNA, 19 nt, Homo sapiens
ggggugaaacuucuaagag

SEQ ID NO: 106 - RNA, 19 nt, Homo sapiens
cucuuagaaguuucacccc

SEQ ID NO: 107 - RNA, 19 nt, Homo sapiens
acuucuaagagaagcaaug

SEQ ID NO: 108 - RNA, 19 nt, Homo sapiens
cauugcuucucuuagaagu

SEQ ID NO: 109 - RNA, 19 nt, Homo sapiens
gagaagcaaugggaauuua

SEQ ID NO: 110 - RNA, 19 nt, Homo sapiens
uaaauucccauugcuucuc

SEQ ID NO: 111 - RNA, 19 nt, Homo sapiens
gcaaugggaauuuacauca

SEQ ID NO: 112 - RNA, 19 nt, Homo sapiens
ugauguaaauucccauugc

SEQ ID NO: 113 - RNA, 19 nt, Homo sapiens
ugggaauuuacaucagcac

SEQ ID NO: 114 - RNA, 19 nt, Homo sapiens
gugcugauguaaauuccca

SEQ ID NO: 115 - RNA, 19 nt, Homo sapiens
uuuacaucagcacccucaa

SEQ ID NO: 116 - RNA, 19 nt, Homo sapiens
uugagggugcugauguaaa

SEQ ID NO: 117 - RNA, 19 nt, Homo sapiens
ugaauggagagucaguaga

SEQ ID NO: 118 - RNA, 19 nt, Homo sapiens
ucuacugacucuccauuca

SEQ ID NO: 119 - RNA, 19 nt, Homo sapiens
uggagagucaguagaccca

SEQ ID NO: 120 - RNA, 19 nt, Homo sapiens
ugggucuacugacucucca

SEQ ID NO: 121 - RNA, 19 nt, Homo sapiens
gccugcuccuucaaaaacc

SEQ ID NO: 122 - RNA, 19 nt, Homo sapiens
gguuuuugaaggagcaggc

SEQ ID NO: 123 - RNA, 19 nt, Homo sapiens
aauccccacuuguaagauc

SEQ ID NO: 124 - RNA, 19 nt, Homo sapiens
gaucuuacaaguggggauu

SEQ ID NO: 125 - RNA, 19 nt, Homo sapiens
uccccacuuguaagaucac

SEQ ID NO: 126 - RNA, 19 nt, Homo sapiens
gugaucuuacaagugggga

SEQ ID NO: 127 - RNA, 19 nt, Homo sapiens
gaucacucuuaaggaaacc

SEQ ID NO: 128 - RNA, 19 nt, Homo sapiens
gguuuccuuaagagugauc

SEQ ID NO: 129 - RNA, 19 nt, Homo sapiens
ggaaaccuuccugacguca

SEQ ID NO: 130 - RNA, 19 nt, Homo sapiens
ugacgucaggaagguuucc

SEQ ID NO: 131 - RNA, 19 nt, Homo sapiens
cauuagaagcagacagagg

SEQ ID NO: 132 - RNA, 19 nt, Homo sapiens
ccucugucugcuucuaaug

SEQ ID NO: 133 - RNA, 19 nt, Homo sapiens
gcagacagagguggaaagu

SEQ ID NO: 134 - RNA, 19 nt, Homo sapiens
acuuuccaccucugucugc

SEQ ID NO: 135 - RNA, 19 nt, Homo sapiens
aguuccacaugguagaugg

SEQ ID NO: 136 - RNA, 19 nt, Homo sapiens
ccaucuaccauguggaacu

SEQ ID NO: 137 - RNA, 19 nt, Homo sapiens
cgucucuggggaauuuacu

SEQ ID NO: 138 - RNA, 19 nt, Homo sapiens
aguaaauuccccagagacg

SEQ ID NO: 139 - RNA, 19 nt, Homo sapiens
uuuacugaucuggucccug

SEQ ID NO: 140 - RNA, 19 nt, Homo sapiens
cagggaccagaucaguaaa

SEQ ID NO: 141 - RNA, 19 nt, Homo sapiens
acauauugugaugaagugg

SEQ ID NO: 142 - RNA, 19 nt, Homo sapiens
ccacuucaucacaauaugu

SEQ ID NO: 143 - RNA, 19 nt, Homo sapiens
guggagguuuaaaucuugg

SEQ ID NO: 144 - RNA, 19 nt, Homo sapiens
ccaagauuuaaaccuccac

SEQ ID NO: 145 - RNA, 19 nt, Homo sapiens
acagaccuuuggcuauggc

SEQ ID NO: 146 - RNA, 19 nt, Homo sapiens
gccauagccaaaggucugu

SEQ ID NO: 147 - DNA, 63 bp, Homo sapiens
gatccattggtccacggataagctttcaagagaagcttatccgtggaccaatttttttggaaa

SEQ ID NO: 148 - DNA, 63 bp, Homo sapiens
agcttttccaaaaaaattggtccacggataagcttctcttgaaagcttatccgtggaccaatg

SEQ ID NO: 149 - DNA, 63 bp, Homo sapiens
gatccgtgagtaagcttgatggagttcaagagactccatcaagcttactcacttttttggaaa

SEQ ID NO: 150 - DNA, 63 bp, Homo sapiens
agcttttccaaaaaagtgagtaagcttgatggagtctcttgaactccatcaagcttactcacg

SEQ ID NO: 151 - DNA, 63 bp, Homo sapiens
gatccactaaactggacaggtactttcaagagaagtacctgtccagtttagtttttttggaaa

SEQ ID NO: 152 - DNA, 63 bp, Homo sapiens
agcttttccaaaaaaactaaactggacaggtacttctcttgaaagtacctgtccagtttagtg